(19) United States
(12) Patent Application Publication
(10) Pub. No.: US 2014/0322220 A1
(43) Pub. Date: Oct. 30, 2014

(54) Anti-FGFR2 Antibodies and Uses Thereof

(71) Applicant: Bayer Intellectual Property GmbH, Monheim (DE)

(72) Inventors: Axel Harrenga, Wuppertal (DE); Charlotte Christine Kopitz, Falkensee (DE); Stefanie Hammer, Berlin (DE); Frank Dittmer, Dusseldorf (DE); Sven Golfier, Berlin (DE); Mark Trautwein, Wulfrath (DE); Sandra Bruder, Leverkusen (DE); Juergen Franz, Witten (DE); Beatrix Stelte-Ludwig, Wulfrath (DE); Lars Linden, Dusseldorf (DE); Ricarda Finnern, Aachen (DE); Simone Greven, Dormagen (DE); Jan Tebbe, Koln (DE)

(73) Assignee: BAYER INTELLECTUAL PROPERTY GMBH, Monheim (DE)

(21) Appl. No.: 14/359,663

(22) PCT Filed: Nov. 22, 2012
(86) PCT No.: PCT/EP2012/073325

§ 371 Date: May 21, 2014

(30) Foreign Application Priority Data
Nov. 23, 2011 (EP) 11190227.6

(51) Int. Cl. C07K 16/28; A61K 45/06; A61K 47/48; A61K 39/395

(52) U.S. Cl. CPC C07K 16/2863; A61K 39/3955; A61K 45/06; A61K 47/48561
USPC 424/135.1; 530/387.9; 530/387.3; 530/391.7; 536/23.53; 424/139.1; 424/178.1; 424/133.1; 435/320.1; 435/69.6; 435/254.2; 435/328; 435/252.3; 435/252.33; 435/252.31; 435/252.34; 435/252.35

(57) ABSTRACT

The present invention provides antibodies, or antigen-binding antibody fragments thereof, or variants thereof which reduce the cell surface expression of FGFR2 after binding to FGFR2 in both cells overexpressing FGFR2 and cells expressing mutated FGFR2. Also provided are antibody-based therapies for FGFR2-related diseases or conditions such as cancer. Antibodies of the invention also can be used in the diagnostics field. The invention also provides nucleic acid sequences encoding the foregoing antibodies, vectors containing the same, pharmaceutical compositions and kits with instructions for use.

The present invention provides recombinant antigen-binding regions and antibodies and functional fragments containing such antigen-binding regions that are specific for the fibroblast growth factor receptor 2 (FGFR2).

The antibodies, accordingly, can be used to treat tumors and other disorders and conditions associated with expression of FGFR2. The invention also provides nucleic acid sequences encoding the foregoing antibodies, vectors containing the same, pharmaceutical compositions and kits with instructions for use.

### BACKGROUND OF THE INVENTION

Antibody-based therapy is proving very effective in the treatment of various cancers, including solid tumors. For example, HERCEPTIN® has been used successfully to treat breast cancer and RITUXAN® is effective in B-cell related cancer types. Central to the development of a successful antibody-based therapy is isolation of antibodies against cell-surface proteins found to be preferentially expressed on tumor cells.

Fibroblast growth factor receptors are tyrosine receptor kinases (RTKs), from which four are known (FGFR1, FGFR2, FGFR3, FGFR4) in mammals. As ligands 22 human fibroblast growth factors (FGFs) are identified (Eswarakumar and Schlessinger, Cytokine & Growth Factor Reviews 2005, 16:139-149; Shimada et al., Proc Natl Acad Sci USA 2001, 98:6500-6505). FGFRs consist of three extracellular immunoglobulin (Ig)-like domains, D1-D3, whereby domains 2 and 3 are required for ligand binding, a single transmembrane domain and a cytoplasmic domain containing the catalytic protein tyrosine kinase core (for a schematic representation see FIG. 1). The extracellular part harbors in addition the acidic box (AB) and the heparin binding site (HBS) (see FIG. 1). An important hallmark of the FGFR family of RTKs is that a variety of alternatively spliced variants exist. Full length FGFR2 is called FGFR2 alpha, while the isoform lacking D1 is termed FGFR2 beta (FIG. 1). Alternative splicing in domain 3 results in two different variants namely FGFR2 IIIb, harboring exons 7 and 8, and FGFR2 IIc, containing exons 7 and 9 (FIG. 1). The latter splicing affects ligand binding, resulting in the specificity pattern. FGFR2 IIc is mainly expressed by mesenchymal cells, FGFR2 IIIb mainly by epithelial cells. FGF7 also known as keratinocyte growth factor (KGF) only binds to FGFR2 IIIb, which is therefore also termed KGFR. Upon binding of FGFs to their receptors, subsequently dimerization and phosphorylation of FGFRs and downstream signaling via FRS-GRB2 docking protein complex to RAS-MAPK signaling cascade and PI3K-AKT signaling cascade occurs. The first signaling cascade is implicated in cell growth and differentiation, the latter in cell survival and fate determination (Katoh and Katoh, Int J Oncol 2006, 29:163-168).

Orchestrated signaling of all four receptors (FGFR1 to FGFR4) and their splice variants via the different FGFs is required for proper organogenesis during embryogenesis (Ornitz et al., Genome Biol 2001, 2:3005). In case of FGFR2, lack of all FGFR2 variants results in defects in placenta and limb bud formation and consequently results in lethality in E10.5. Specific knock-out of FGFR2 IIIb also results in lethality (in P0), associated with agenesis of lungs, anterior pituitary, thyroid, teeth and limbs, while disruption of the FGFR2 IIIc variant is viable showing delayed ossification, proportionate dwarfism, and synostosis of skull base (Eswarakumar and Schlessinger, 2005). Germline activating mutations of FGFR2 in humans lead to severe deformities during enbryogenesis, such as coronal- and craniosynostosis in Apert or Pfeiffer syndromes (Robin et al., in Gene Reviews, NCBI Bookshelf Washington, edts. Pagan et al., 1993). In the adult, FGFR2 signaling is involved in wound healing, epithelial repair and cytoprotection of skin and mucosa (Braun et al., Phil Trans R Soc Lond B 2004, 359:753-757) and in regeneration of injured liver (Steiling et al., Oncogene 2003, 22:4380-4388; Böhm, dissertation, Swiss Federal Institute of Technology Zurich, 2009). A role of FGFR2 signaling in migration of epicardial derived cells (EPDCs) into the heart after infarction is under discussion, since during embryogenesis FGF10/FGFR2 signaling is necessary for migration of EPDCs in the compact myocardium, a process required for intact heart development (Vega-Hernández et al., Development 2011:3331-3340; Winter and De Groot, Cell Mol Life Sci 2007, 64:692-703).

Increased, germline-independent signaling through FGFR2 is involved in different pathologies, such as acne (Katoh, J of Invest Dermatol 2009, 129:1861-1867), psoriasis (Finch et al., Am J Pathol 1997, 151:1619-1628; Xu et al., J Invest Dermatol 2011:131:1521-1529) periodontitis (Li et al., J Peridontal Res 2005, 40:128-138), solar lentigines (Lin et al., Journal Dermatol Sci 2010, 59:91-97), bowel disease (Brauchle et al., J Pathol 1996, 149:521-529), endometriosis (Taniguchi et al., Fertil Steril 2008, 89:478-480), cholesteatoma (Yamamoto-Fukuda et al., Eur Arch Otorhinolaryngol (2008) 265:1173-1178; d'Alessandro et al., Otol Neurotol. 2010 Sep.; 31(7):1163-9), cholesteatomatous chronic otitis media (Yamamoto-Fukuda et al., Otol Neurotol. 2010 Jul.; 31(5):745-51), atherosclerosis (Che et al., Am J Physiol Heart Circ Physiol 300: H154-H161, 2011) and cancer (see below).

Several studies are published emphasizing a strong association of FGFR2 expression and poor outcome of cancer patients:

Overexpression of FGFR2 and/or KGF is associated with expansive growth of gastric cancer and shorter survival of patients (Matsunobu et al., Int J Cancer 2006, 28:307-314; Toyokawa et al., Oncol Reports 2009, 21:875-880). Overexpression of FGFR2 was thereby detected in 31-36.5% of all gastric cancer samples tested (Matsunobu et al., Int J Cancer 2006, 28:307-314; Toyokawa et al., Oncol Reports 2009, 21:875-880). Adenocarcinoma (70% of all gastric cancer) are further divided into two distinct pathological types, namely the intestinal- and the diffuse-type gastric cancer. Interestingly, the first, less aggressive type is associated with an activated ErbB2 oncogenic pathway, while the latter, more aggressive phenotype harbors aberrations in the FGFR2/PI3K pathway (Yamashita et al., Surg Today 2011, 41:24-38). Approximately 60% of gastric adenocarcinoma belong to the diffuse-type, the remaining 40% to the intestinal type (Werner et al., J Cancer Res Clin Oncol 2001, 127:207-216). FGFR2 overexpression was found in 53% of diffuse-type gastric cancer samples (Yamashita et al., Surg Today 2011, 41:24-38). Taking all data together, HER2 and FGFR2 expression seem to occur in two distinct patient populations. Possibly, expression of FGFR2 partly results from gene amplification as in approximately 7-10% of primary gastric cancers amplification of FGFR2 can be found (Kunii et al, Cancer Res 2008, 68:23-40-2348). Furthermore, FGFR2 expression was not only found in metastases, but was even stronger than in primary tumors (Yamashita et al., Surg Today 2011, 41:24-38).

In breast cancer, FGFR2 IIIb expression was found in 57% of tumor samples but hardly in healthy tissue (Tamaru et al. 2004, 84:1460-1471). KGF (FGF7) was found in 45% of samples, generally coincided with FGFR2 IIIb. Co-Expression of FGF7 and its only receptor FGFR2 IIIb was associated with a significantly reduced number of apoptotic cells within the primary tumor as compared to primary breast cancers neither expressing FGF7 nor FGFR2 IIIb (Tamaru et al. 2004, 84:1460-1471). As in gastric cancer, also in breast cancer gene amplification was found: in 4% of triple negative breast cancer (TNBC) (Turner et al. Oncogene 2010, 29:2013-2023). In breast cancer several small nuclear polymorphisms (SNPs) were identified, which are associated with increased breast cancer risk (Hunter et al. Nature Genetics 2007, 6:870-874). If SNPs are localized within intron 2, it results in transcriptional up-regulation of FGFR2 (Katoh Expert Reviews 2010, 10:1375-1379). Interestingly, FGFR1 is preferentially upregulated in ER-positive, while FGFR2 in ER-negative breast cancers (Katoh, Expert Reviews 2010, 10:1375-1379)

In pancreatic cancer, overexpression of FGFR2 IIIb and/or FGF7 is strongly correlated with venous invasion (Cho et al., Am J Pathol 170:1964-1974), whereby co-expression of FGFR2 and FGF7 was found in tumor cells, but even more abundant in the stromal cells adjacent to tumor cells (Ishiwata et al., Am J Pathol 1998, 153:213-222).

In epithelial ovarian cancer in 80% of tested cases up-regulation of FGFR2 as compared to normal tissue and in 70% FGF7 in the ascetic fluid was found (Steele et al., Oncogene 20:5878-5887).

FGFR2 protein was found in all tested invasive cervical cancers with strong expression at the invasive front of tumors (Kawase et al., Int J Oncol 2010, 36:331-340).

In lung adenocarcinoma, co-expression of FGF7 and FGFR2 was found in 51.6% of tested cases and correlates with lower differentiation grades, higher proliferation rate, lymph node metastasis and shorter 5-year survival (Yamayoshi et al., J Pathol 2004, 204:110-118).

In endometrial cancer, most frequently activating mutations of FGFR2 are found in approximately 16% of endometrial cancer (Pollock et al., Oncogene 2007, 26:7158-7162).

In esophageal carcinoma (EC), co-expression of FGF7 and FGFR2 in cancer cells was found in 26% of patients associated with a trend for shorter survival (Yoshino et al., Int J Oncol 2007, 31:721-728).

In hepatocellular carcinoma, FGFR2 expression was up-regulated by 4.7 times in poorly differentiated tumors. This expression is associated with incidence of portal vein invasion and lower disease free survival times (Harimoto et al., Oncology 2010, 78:361-368).

Several publications with experimental in vitro and in vivo data demonstrate a causal relationship of aberrant FGFR2-signaling and tumor progression:

Knock-down and/or inhibition of FGFR2 in gastric (Takeda et al., Clin Cancer Res 2007; 13:3051-3057; Kunii et al., Cancer Res 2008; 68:2340-2348), breast (Turner et al. Oncogene 2010, 29:2013-2023), ovarian (Cole et al., Cancer Biol Ther 2010, 10:495-504) and head and neck squamous cell (Marshall et al., Clin Cancer Res 2011, 17:5016-5025) carcinoma cells resulted in reduced proliferation and/or increased apoptosis of tumor cells. Also in tumor xenografts, knock-down of FGFR2 as well as inhibition of FGFR2 in tumor cell lines over-expressing FGFR2, growth inhibition was shown for gastric (Takeda et al., Clin Cancer Res 2007; 13:3051-3057) and ovarian (Cole et al., Cancer Biol Ther 2010, 10:495-504) cancer cell lines. Additionally, FGF7, which solely activates FGFR2, increases proliferation of gastric (Shin et al., J Cancer Res Clin Oncol 2002, 128:596-602), breast (Zhang et al., Anticancer Res 1998, 18:2541-2546) and ovarian (Cole et al., Cancer Biol Ther 2010, 10:495-504) cancer cell lines in vitro and in vivo. Furthermore, knock-down of FGFR2 in endometrial cancer cell lines harboring FGFR2 with activating mutations also resulted in cell cycle arrest and induction of cell death (Byron et al., Cancer Res 2008, 68:6902-6907).

FGFR2 signaling promotes migration and invasion of gastric (Shin et al., J Cancer Res Clin Oncol 2002, 128:596-602), breast (Zhang et al., Anticancer Res 1998, 18:2541-2546) and pancreatic cancer cell lines in vitro (Nomura et al., Br J Cancer 2008, 99:305-313; Niu et al., J Biol Chem 2007, 282:6601-6011).

In esophageal carcinoma, FGFR2 is the highest up-regulated gene in tumor-associated fibroblasts. Isolated tumor-associated fibroblasts released a soluble factor that promotes proliferation of esophageal cancer cells (Zhang et al., hum Cancer Biol 2009, 15:4017-4022), demonstrating that also FGFR2 expressed by stromal cells can promote tumor progression.

Only a limited number of anti FGFR2 antibodies have been reported. Fortin et al. (J. Neurosci. 2005, 25: 7470-7479) describe a blocking anti FGFR2 antibody. Wei et al. (Hybridoma 2006, 25: 115-124) showed antibodies specific only for FGFR2 IIIb that inhibits KGF induced cell proliferation. In WO2007/144893 inhibitory antibodies that bind FGFR2 and FGFR3 are disclosed. In WO2010/054265 and Zhao et al. (Clin Cancer Res. 2010, 16:5750-5758) antibodies inhibiting FGF binding are disclosed. Bai et al. (Cancer Res. 2010, 70:7630-7639) describe antibodies specific for FGFR2 IIIb. R&D Systems markets anti-FGFR2 antibodies that neutralize activity in their assays.

In summary, several FGFR2 splice variants are known. Furthermore, it is known that FGFR2-related diseases are due to aberrant expression, e.g. overexpression or amplification of FGFR2, or due to various mutated FGFR2 proteins. However, a therapy is lacking which addresses a plurality of different FGFR2 related diseases.

### SUMMARY OF THE INVENTION

The present invention is directed to the provision of antibodies, or antigen-binding antibody fragments thereof, or variants thereof which reduce the cell surface expression of FGFR2 after binding to FGFR2 in both cells overexpressing FGFR2 and cells expressing mutated FGFR2. Also provided are antibody-based therapies for FGFR2-related diseases or conditions such as cancer, in particular for FGFR2 expressing tumors, such as gastric cancer, breast cancer, pancreatic cancer, colorectal cancer, renal cell carcinoma, prostate cancer, ovarian cancer, cervical cancer, lung cancer, non-small-cell lung cancer (NSCLC), endometrial cancer, esophageal cancer, head and neck cancer, hepatocellular carcinoma, melanoma and bladder cancer.

The invention is also related to polynucleotides encoding the antibodies of the invention, or antigen-binding fragments thereof, cells expressing the antibodies of the invention, or antigen-binding fragments thereof, methods for producing the antibodies of the invention, or antigen-binding fragments thereof, methods for inhibiting the growth of dysplastic cells using the antibodies of the invention, or antigen-binding fragments thereof, and methods for treating and detecting cancer using the antibodies of the invention, or antigen-binding fragments thereof.

The invention describes antibodies that are distinguished from existing FGFR2 antibodies in that they reduce the surface expression of FGFR2 after binding to FGFR2 in cells overexpressing FGFR2 as well as in cells expressing mutated FGFR2. An embodiment of the invention is an antibody or antigen-binding fragment thereof that binds to the extracellular N-terminal epitope (¹RPSFSLVEDTTLEPE¹⁵) of FGFR2 (SEQ ID NO:63). The antibodies or antigen-binding fragment thereof of the invention a) activate FGFR2 on the short term, b) induce internalization of FGFR2 c) resulting in efficient degradation, d) de-sensibilization of the FGFR2-expressing cancer cells or tumor cells and e) finally resulting in an anti-tumor activity of these antibodies in in vivo tumor experiments. These and other objects of the invention are more fully described herein.

An antibody of the invention might be co-administered with known medicaments, and in some instances the antibody might itself be modified. For example, an antibody could be conjugated to a cytotoxic agent, immunotoxin, toxophore or radioisotope to potentially further increase efficacy.

The invention further provides antibodies which constitute a tool for diagnosis of malignant or dysplastic conditions in which FGFR2 expression is elevated compared to normal tissue or where FGFR2 is shed from the cell surface and becoming detectable in serum. Provided are anti-FGFR2 antibodies conjugated to a detectable marker. Preferred markers are a radiolabel, an enzyme, a chromophore or a fluorescer.

The invention is also related to polynucleotides encoding the antibodies of the invention, or antigen-binding fragments thereof, cells expressing the antibodies of the invention, or antigen-binding fragments thereof, methods for producing the antibodies of the invention, or antigen-binding fragments thereof, methods for inhibiting the growth of dysplastic cells using the antibodies of the invention, or antigen-binding fragments thereof, and methods for treating and detecting cancer using the antibodies of the invention, or antigen-binding fragments thereof.

The invention also is related to isolated nucleic acid sequences, each of which can encode an aforementioned antibody or antigen-binding fragment thereof that is specific for an epitope of FGFR2. Nucleic acids of the invention are suitable for recombinant production of antibodies or antigen-binding antibody fragments. Thus, the invention also relates to vectors and host cells containing a nucleic acid sequence of the invention.

Compositions of the invention may be used for therapeutic or prophylactic applications. The invention, therefore, includes a pharmaceutical composition comprising an inventive antibody or antigen-binding fragment thereof and a pharmaceutically acceptable carrier or excipient therefore. In a related aspect, the invention provides a method for treating a disorder or condition associated with the undesired presence of FGFR2 expressing cells. In a preferred embodiment the aforementioned disorder is cancer. Such method contains the steps of administering to a subject in need thereof an effective amount of the pharmaceutical composition that contains an inventive antibody as described or contemplated herein.

The invention also provides instructions for using an antibody library to isolate one or more members of such library that binds specifically to FGFR2.

### DESCRIPTION OF THE FIGURES

FIG. 1: Schematic diagram of the structure of FGFR2. Alpha (SEQ ID NO:61) and beta (SEQ ID NO:62) splice variants are shown in comparison. The diagram shows the three Ig-like domains (D1, D2 and D3), the transmembrane domain (TM), and the intracellular kinase domain. The heparin binding site (FIBS), acidic box (AB), and the alternative IIIb/IIIc partial domains are indicated. The amino terminus is marked by an N, the carboxy terminus by an C. The binding epitope of the antibodies of this invention is depicted striped.

FIG. 2: Induction of phosphorylated FGFR2 (P-FGFR2) levels after short term (15 min) incubation with anti FGFR2 antibodies at 10 μg/ml in MFM223 cells. Y is “% of untreated control cells”. As shown antibodies M048-D01-hIgG1 and M047-D08-hIgG1 increase the ELISA signal of P-FGFR2 by a factor greater 4 fold compared with untreated control cells. In contrast neither the control IgG antibody nor anti FGFR2 antibodies commercially available from R&D (MAB665, MAB684, MAB6843) showed any significant effect on P-FGFR2 levels after short-term incubation. These results reveal an agonistic effect of anti FGFR2 antibodies described within this invention on FGFR2 after short-term incubation.

FIG. 3: Desensitizing of MFM223 cells against FGF7 (25 ng/ml, 15 min) mediated induction of P-FGFR2 levels after long term (24 h) incubation with anti FGFR2 antibodies at 10 μg/ml. Y is “% of untreated control cells”. As shown the antibodies M048-D01-hIgG1 and M047-D08-hIgG1 reduce the level of P-FGFR2 which can be achieved after FGF7 stimulation very pronounced. In cells treated without antibody treatment as well as in cells treated with isotype control IgG stimulation with FGF7 lead to an about 4 fold increase of P-FGFR2 levels. In contrast, in samples pretreated with anti FGFR2 antibodies for 24 h, FGF7 only induced P-FGFR2 levels by 1.37-1.4 fold. Taken together these results show that prolonged incubation of cells with anti FGFR2 antibodies of this invention leads to desensitization towards stimulation with FGF7.

FIG. 4: Downregulation of FGFR2 surface expression in cell lines with FGFR2 overexpression (MFM223, SNU16) or FGFR2 mutations (AN3-CA, MFE-296) 4.5 h after incubation with anti FGFR2 antibodies at 10 μg/ml measured by FACS analysis. Y is “% of control cells”. As shown antibodies M048-D01-hIgG1 and M047-D08-hIgG1 are the only antibodies that reduce FGFR2 surface expression with FGFR2 overexpressing cell lines (MFM223, SNU16) and cells lines having FGFR2 mutations (AN3-CA, MFE-296). Antibodies like MAB684 and MAB6843 (R&D) only reduce FGFR2 surface expression with cell lines which do not overexpress FGFR2. Antibodies like GAL-FR21 do not reduce FGFR2 surface expression with cell lines having FGFR2 mutations.

FIG. 5: Downregulation of total FGFR2 levels after long term (96 h) incubation with anti FGFR2 antibodies in SNU16 cells. Y is “% of control cells”. X is “Antibody concentration [μg/ml]”. As shown antibodies M048-D01-hIgG1 (white) and M047-D08-hIgG1 (striped) decrease the total FGFR2 levels significantly after 96 h in a dose dependent manner. A non-binding control antibody (black) does not show any effects. These results indicate that anti FGFR2 antibodies M048-D01-hIgG1 and M047-D08-hIgG1 do not only lead to a short term decrease in surface FGFR2 levels but also a long term reduction of total FGFR2 levels.

FIG. 6: Microscopic evaluation of the time course of specific internalization of M048-D01-hIgG1 and M047-D08-hIgG1 upon binding to endogenous FGFR2 expressing cells. Y is “granule counts per cell”. X is “time [min]”. Internalization of antibodies was investigated on breast cancer cell line SUM 52PE. The granule counts per cell were measured in a kinetic fashion. As shown antibodies M048-D01-hIgG1 (black squares and solid line) and M047-D08-hIgG1 (black triangles and dashed line) show a rapid internalization as indicated by increasing granule count per cell. An isotype control antibody (stars and dashed line) does not show any internalization.

FIG. 7: Internalization of M048-D01-hIgG1 (A, B) and M047-D08-hIgG1 (C, D) in SUM 52PE cells showed co-staining as indicated with Rab 7 (A, C) and not with Rab 11 (B, D). Internalization of GAL-FR21 (E, F) and GAL-FR22 (G,H) in SUM 52PE cells showed co-staining as indicated with Rab 11 (F, H) and not with Rab 7 (E, G).

FIG. 8: Growth of subcutaneous SNU-16 xenografts under intraperitoneal treatment with 2 mg/kg of M017-B02-hIgG1 (open triangles, solid line) in comparison to PBS (filled circles, solid line) and control IgG treatment (filled triangles, solid line). Mean+standard deviation are plotted. X is “time after tumor inoculation [days]”. Y is “tumor area [mm²]”. Treatment with M017-B02-hIgG1 resulted in a very significant tumor growth inhibition.

FIG. 9: Growth of subcutaneous SNU-16 xenografts under intraperitoneal treatment with 2 mg/kg of M021-H02-hIgG1 (open triangles, solid line) in comparison to PBS (filled circles, solid line) and control IgG treatment (filled triangles, solid line). Mean+standard deviation are plotted. X is “time after tumor inoculation [days]”. Y is “tumor area [mm]”. Treatment with M021-H02-hIgG1 resulted in a very significant tumor growth inhibition.

FIG. 10: Growth of subcutaneous SNU-16 xenografts under intraperitoneal treatment with 2 mg/kg of M048-D01-hIgG1 (open triangles, solid line) in comparison to PBS (filled circles, solid line) and control IgG treatment (filled triangles, solid line). Mean+standard deviation are plotted. X is “time after tumor inoculation [days]”. Y is “tumor area [mm²]”. Treatment with M048-D01-hIgG1 resulted in a very significant tumor growth inhibition.

FIG. 11: Growth of subcutaneous SNU-16 xenografts under intraperitoneal treatment with 2 mg/kg of M054-A05-hIgG1 (open triangles, solid line) in comparison to PBS (filled circles, solid line) and control IgG treatment (filled triangles, solid line). Mean+standard deviation are plotted. X is “time after tumor inoculation [days]”. Y is “tumor area [mm²]”. Treatment with M054-A05-hIgG1 resulted in a very significant tumor growth inhibition.

FIG. 12: Growth of subcutaneous SNU-16 xenografts under intraperitoneal treatment with 2 mg/kg of M054-D03-hIgG1 (open triangles, solid line) in comparison to PBS (filled circles, solid line). Mean+standard deviation are plotted. X is “time after tumor inoculation [days]”. Y is “tumor area [mm²]”. Treatment with M054-D03-hIgG1 resulted in a very significant tumor growth inhibition.

FIG. 13: Growth of subcutaneous SNU-16 xenografts under intraperitoneal treatment with 2 mg/kg of M047-D08-hIgG1 (open triangles, solid line) in comparison to PBS (filled circles, solid line). Mean+standard deviation are plotted. X is “time after tumor inoculation [days]”. Y is “tumor area [mm²]”. Treatment with M047-D08-hIgG1 resulted in a very significant tumor growth inhibition.

FIG. 14: Dot plots of the tumor area of subcutaneous 4T1 tumors at day 13 after tumor cell inoculation, the last time point before tumors became necrotic. At this time point mice received treatment with PBS alone (A), 5 mg/kg of M048-D01-hIgG1 twice weekly i.v. (B), 100 mg/kg Lapatinib p.o. (C) or with 5 mg/kg of M048-D01-hIgG1 twice weekly i.v. and 100 mg/kg Lapatinib p.o. (D). Y is tumor area [mm²] at day 13, dotted lines indicate the mean values, solid lines indicate the medians. Treatment with M048-D01-hIgG1 alone resulted in a significant reduction of tumor area, while Lapatinib alone did not significantly affect tumor area. Combination of M048-D01-hIgG1 with Lapatinib resulted in a significantly additive anti-tumor activity.

FIG. 15: Dot plots of the tumor area of subcutaneous 4T1 tumors at day 13 after tumor cell inoculation, the last time point before tumors became necrotic. At this time point mice received treatment with PBS alone (A), 5 mg/kg of M048-D01-hIgG1 twice weekly i.v. (B), 24 mg/kg Taxol once weekly i.v. (C) or with 5 mg/kg of M048-D01-hIgG1 twice weekly i.v. and 24 mg/kg Taxol once weekly i.v. (D). Y is tumor area [mm²] at day 13, dotted lines indicate the mean values, solid lines indicate the medians. Treatment with M048-D01-hIgG1 alone resulted in a significant reduction of tumor area, while Taxol alone did not significantly affect tumor area. Combination of M048-D01-hIgG1 with Taxol resulted in a significantly additive anti-tumor activity.

FIG. 16: Growth of subcutaneous patient-derived GC10-0608 xenografts under intraperitoneal treatment with 5 mg/kg (filled triangles, solid line), 2 mg/kg (filled circles, dashed line) and 1 mg/kg (filled squares, dotted line) of M048-D01-hIgG1 in comparison to PBS (open diamonds, solid line). Mean±standard error of the means are plotted. X is “time under treatment [days]”. Y is “tumor volume [mm³]”. Treatment with all three doses of M048-D01-hIgG1 resulted in a significant tumor growth inhibition.

FIG. 17: Growth of subcutaneous patient-derived. GC12-0811 xenografts under intraperitoneal treatment with 5 mg/kg (filled triangles, solid line), 2 mg/kg (filled circles, dashed line) and 1 mg/kg (filled squares, dotted line) of M048-D01-hIgG1 in comparison to PBS (open diamonds, solid line). Mean±standard error of the means are plotted. X is “time under treatment [days]”. Y is “tumor volume [mm³]”. Treatment with doses of 5 and 1 mg/kg M048-D01-hIgG1 resulted in a significant tumor growth inhibition.

FIG. 18: Downregulation of total FGFR2 [total FGFR2] and phosphorylated FGFR2 [P-FGFR2] after long term treatment of SNU16 xenografts with anti FGFR2 antibodies M048-D01-hIgG1 and M047-D08-hIgG1 in comparison with a control antibody (2 mg/kg, twice weekly, i.p., samples were taken 24 h after the last dose). As shown after treatment with M048-D01-hIgG1 and M047-D08-hIgG1 total FGFR2 [total FGFR2] and phosphorylated FGFR2 [P-FGFR2] were reduced significantly in comparison with treatment with control IgG1. Actin served as loading control.

FIG. 19: Sequences of the invention

### DETAILED DESCRIPTION OF THE INVENTION

The present invention is based on the discovery of novel antibodies that have a specific affinity for FGFR2 and can deliver a therapeutic benefit to a subject. The antibodies of the invention, which may be human, humanized or chimeric, can be used in many contexts, which are more fully described herein.

### DEFINITIONS

Unless defined otherwise, all technical and scientific terms used herein have the meaning commonly understood by one of ordinary skill in the art to which this invention belongs. The following references, however, can provide one of skill in the art to which this invention pertains with a general definition of many of the terms used in this invention, and can be referenced and used so long as such definitions are consistent the meaning commonly understood in the art. Such references include, but are not limited to, Singleton et ah, Dictionary of Microbiology and Molecular Biology (2d ed. 1994); The Cambridge Dictionary of Science and Technology (Walker ed., 1988); Hale & Marham, The Harper Collins Dictionary of Biology (1991); and Lackie et al., The Dictionary of Cell & Molecular Biology (3d ed. 1999); and Cellular and Molecular Immunology, Eds. Abbas, Lichtman and Pober, 2nd Edition, W.B. Saunders Company. Any additional technical resources available to the person of ordinary skill in the art providing definitions of terms used herein having the meaning commonly understood in the art can be consulted. For the purposes of the present invention, the following terms are further defined. Additional terms are defined elsewhere in the description. As used herein and in the appended claims, the singular forms “a,” “and,” and “the” include plural reference unless the context clearly dictates otherwise. Thus, for example, reference to “a gene” is a reference to one or more genes and includes equivalents thereof known to those skilled in the art, and so forth.

A “human” antibody or antigen-binding fragment thereof is hereby defined as one that is not chimeric (e.g., not “humanized”) and not from (either in whole or in part) a non-human species. A human antibody or antigen-binding fragment thereof can be derived from a human or can be a synthetic human antibody. A “synthetic human antibody” is defined herein as an antibody having a sequence derived, in whole or in part, in silico from synthetic sequences that are based on the analysis of known human antibody sequences. In silico design of a human antibody sequence or fragment thereof can be achieved, for example, by analyzing a database of human antibody or antibody fragment sequences and devising a polypeptide sequence utilizing the data obtained there from. Another example of a human antibody or antigen-binding fragment thereof is one that is encoded by a nucleic acid isolated from a library of antibody sequences of human origin (e.g., such library being based on antibodies taken from a human natural source). Examples of human antibodies include antibodies as described in Söderlind et al., Nature Biotech. 2000, 18:853-856.

A “humanized antibody” or humanized antigen-binding fragment thereof is defined herein as one that is (i) derived from a non-human source (e.g., a transgenic mouse which bears a heterologous immune system), which antibody is based on a human germline sequence; (ii) where amino acids of the framework regions of a non human antibody are partially exchanged to human amino acid sequences by genetic engineering or (iii) CDR-grafted, wherein the CDRs of the variable domain are from a non-human origin, while one or more frameworks of the variable domain are of human origin and the constant domain (if any) is of human origin.

A “chimeric antibody” or antigen-binding fragment thereof is defined herein as one, wherein the variable domains are derived from a non-human origin and some or all constant domains are derived from a human origin.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the term “monoclonal” indicates the character of the antibody as not being a mixture of discrete antibodies. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. The term “monoclonal” is not to be construed as to require production of the antibody by any particular method. The term monoclonal antibody specifically includes chimeric, humanized and human antibodies.

As used herein, an antibody “binds specifically to”, is “specific to/for” or “specifically recognizes” an antigen of interest, e.g. a tumor-associated polypeptide antigen target (here, FGFR2), is one that binds the antigen with sufficient affinity such that the antibody is useful as a therapeutic agent in targeting a cell or tissue expressing the antigen, and does not significantly cross-react with other proteins or does not significantly cross-react with proteins other than orthologs and variants (e.g. mutant forms, splice variants, or proteolytically truncated forms) of the aforementioned antigen target. The term “specifically recognizes” or “binds specifically to” or is “specific to/for” a particular polypeptide or an epitope on a particular polypeptide target as used herein can be exhibited, for example, by an antibody, or antigen-binding fragment thereof, having a monovalent K_{D} for the antigen of less than about 10⁻⁴ M, alternatively less than about 10⁻⁵ M, alternatively less than about 10⁻⁶ M, alternatively less than about 10⁻⁷ M, alternatively less than about 10⁻⁸ M, alternatively less than about 10⁻⁹ M, alternatively less than about 10⁻¹⁰ M, alternatively less than about 10⁻¹¹ M, alternatively less than about 10⁻¹² M, or less. An antibody “binds specifically to,” is “specific to/for” or “specifically recognizes” an antigen if such antibody is able to discriminate between such antigen and one or more reference antigen(s). In its most general form, “specific binding”. “binds specifically to”, is “specific to/for” or “specifically recognizes” is referring to the ability of the antibody to discriminate between the antigen of interest and an unrelated antigen, as determined, for example, in accordance with one of the following methods. Such methods comprise, but are not limited to Western blots, ELISA-, RIA-, ECL-, IRMA-tests and peptide scans. For example, a standard ELISA assay can be carried out. The scoring may be carried out by standard color development (e.g. secondary antibody with horseradish peroxidase and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative is more than 5-fold, 10-fold, 50-fold, and preferably more than 100-fold. Typically, determination of binding specificity is performed by using not a single reference antigen, but a set of about three to five unrelated antigens, such as milk powder, BSA, transferrin or the like.

“Binding affinity” refers to the strength of the sum total of noncovalent interactions between a single binding site of a molecule and its binding partner. Unless indicated otherwise, as used herein, “binding affinity” refers to intrinsic binding affinity which reflects a 1:1 interaction between members of a binding pair (e.g. an antibody and an antigen). The dissociation constant “K_{D}” is commonly used to describe the affinity between a molecule (such as an antibody) and its binding partner (such as an antigen) i.e. how tightly a ligand binds to a particular protein. Ligand-protein affinities are influenced by noncovalent intermolecular interactions between the two molecules Affinity can be measured by common methods known in the art, including those described herein. In one embodiment, the “K_{D}” or “K_{D} value” according to this invention is measured by using surface plasmon resonance assays using a Biacore T100 instrument (GE Healthcare Biacore, Inc.) according to Example 7. In brief, antibodies were immobilized onto a CM5 sensor chip through an indirect capturing reagent, anti-human IgG Fc. Reagents from the “Human Antibody Capture Kit” (BR-1008-39, GE Healthcare Biacore, Inc.) were used as described by the manufacturer. Approximately 5000 resonance units (RU) monoclonal mouse anti-human IgG (Fc) antibody were immobilized per cell. Anti FGFR2 antibodies were injected to reach a capturing level of approximately 200 to 600 RU. Various concentrations of human, murin, rat, dog and of other species derived FGFR2 peptides containing amino acids 1-15 were injected over immobilized anti-FGFR2 antibodies. Sensograms were generated after in-line reference cell correction followed by buffer sample subtraction. The dissociation equilibrium constant (K_{D}) was calculated based on/be ratio of association (kₒₙ) and dissociation rated (k_{off}) constants, obtained by fitting sensograms with a first order 1:1 binding model using Biacore Evaluation Software. Other suitable devices are BIACORE(R)-2000, a BIACORE (R)-3000 (BIAcore, Inc., Piscataway, N.J.), or ProteOn XPR36 instrument (Bio-Rad Laboratories, Inc.).

To determine critical residues for binding of the antibodies or antibody fragments epitope fine mapping can be performed, using for example Alanine-scanning of peptides. Therefore, each amino acid of the binding epitope is replaced by an Alanine residue and the binding of representative antibodies of the invention is tested in an ELISA-based assay. Thereby, a residue is regarded as critical for binding when the antibody loses more than 50% of its ELISA signal by changing this residue into an Alanine as described in example 6.

The term “antibody”, as used herein, is intended to refer to immunglobulin molecules, preferably comprised of four polypeptide chains, two heavy (H) chains and two light (L) chains which are typically inter-connected by disulfide bonds. Each heavy chain is comprised of a heavy chain variable region (abbreviated herein as VH) and a heavy chain constant region. The heavy chain constant region can comprise e.g. three domains CH1, CH2 and CH3. Each light chain is comprised of a light chain variable region (abbreviated herein as VL) and a light chain constant region. The light chain constant region is comprised of one domain (CL). The VH and VL regions can be further subdivided into regions of hypervariability, termed complementarity determining regions (CDR), interspersed with regions that are more conserved, termed framework regions (FR). Each VH and VL is typically composed of three CDRs and up to four FRs. arranged from amino terminus to carboxy-terminus e.g. in the following order: FR1, CDR1, FR2, CDR2, FR3, CDR3, FR4.

As used herein, the term “Complementarity Determining Regions (CDRs; e.g., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (e.g. about residues 24-34 (L1), 50-56 (L2) and 89-97 (L3) in the light chain variable domain and 31-35 (H1), 50-65 (H2) and 95-102 (H13) in the heavy chain variable domain; (Kabat et al., Sequences of Proteins of Immulological Interest, 5th Ed. Public Health Service, National Institutes of Health, Bethesda, Md. (1991)) and/or those residues from a “hypervariable loop” (e.g. about residues 26-32 (L1), 50-52 (L2) and 91-96 (L3) in the light chain variable domain and 26-32 (H1), 53-55 (H2) and 96-101 (H3) in the heavy chain variable domain (Chothia and Lesk; J Mol Biol 196: 901-917 (1987)). In some instances, a complementarity determining region can include amino acids from both a CDR region defined according to Kabat and a hypervariable loop.

Depending on the amino acid sequence of the constant domain of their heavy chains, intact antibodies can be assigned to different “classes”. There are five major classes of intact antibodies: IgA, IgD, IgE, IgG, and IgM, and several of these maybe further divided into “subclasses” (isotypes), e.g., IgG1, IgG2, IgG3, IgG4, IgA, and IgA2. The heavy-chain constant domains that correspond to the different classes of antibodies are called [alpha], [delta], [epsilon], [gamma], and [mu], respectively. The subunit structures and three-dimensional configurations of different classes of immunglobulins are well known. As used herein antibodies are conventionally known antibodies and functional fragments thereof.

A “functional fragment” or “antigen-binding antibody fragment” of an antibody/immunoglobulin hereby is defined as a fragment of an antibody/immunoglobulin (e.g., a variable region of an IgG) that retains the antigen-binding region. An “antigen-binding region” of an antibody typically is found in one or more hyper variable region(s) of an antibody, e.g., the CDR1, −2, and/or −3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and 1 to 113 of VH; numbering according to WO 97/08320). A preferred class of immunoglobulins for use in the present invention is IgG.

“Functional fragments” or “antigen-binding antibody fragments” of the invention include Fab, Fab′, F(ab′)₂, and Fv fragments; diabodies; single domain antibodies (DAbs), linear antibodies; single-chain antibody molecules (scFv); and multispecific, such as bi- and tri-specific, antibodies formed from antibody fragments (C. A. K Borrebaeck, editor (1995) Antibody Engineering (Breakthroughs in Molecular Biology), Oxford University Press; R. Kontermann & S. Duebel, editors (2001) Antibody Engineering (Springer Laboratory Manual), Springer Verlag). An antibody other than a “multi-specific” or “multi-functional” antibody is understood to have each of its binding sites identical. The F(ab′)2 or Fab may be engineered to minimize or completely remove the intermolecular disulphide interactions that occur between the C_{H1} and C_{L} domains.

Variants of the antibodies or antigen-binding antibody fragments contemplated in the invention are molecules in which the binding activity of the antibody or antigen-binding antibody fragment for FGFR2 is maintained.

Binding proteins contemplated in the invention are for example antibody mimetics, such as Affibodies, Adnectins, Anticalins, DARPins, Avimers, Nanobodies (reviewed by Gebauer M. et al., Curr. Opinion in Chem. Biol. 2009; 13:245-255; Nuttall S. D. et al., Curr. Opinion in Pharmacology 2008; 8:608-617).

As used herein, the term ‘epitope’ includes any protein determinant capable of specific binding to an immunoglobulin or T-cell receptors. Epitopic determinants usually consist of chemically active surface groupings of molecules such as amino acids or sugar side chains, or combinations thereof and usually have specific three dimensional structural characteristics, as well as specific charge characteristics. Two antibodies are said to ‘bind the same epitope’ if one antibody is shown to compete with the second antibody in a competitive binding assay, by any of the methods well known to those of skill in the art.

An “isolated” antibody is one that has been identified and separated from a component of the cell that expressed it. Contaminant components of the cell are materials that would interfere with diagnostic or therapeutic uses of the antibody, and may include enzymes, hormones, and other proteinaceous or nonproteinaceous solutes. In preferred embodiments, the antibody is purified (1) to greater than 95% by weight of antibody as determined e.g. by the Lowry method, UV-Vis spectroscopy or by SDS-Capillary Gel electrophoresis (for example on a Caliper LabChip GXII, GX 90 or Biorad Bioanalyzer device), and in further preferred embodiments more than 99% by weight, (2) to a degree sufficient to obtain at least 15 residues of N-terminal or internal amino acid sequence, or (3) to homogeneity by SDS-PAGE under reducing or nonreducing conditions using Coomassie blue or, preferably, silver stain. Isolated naturally occurring antibody includes the antibody in situ within recombinant cells since at least one component of the antibody's natural environment will not be present. Ordinarily, however, isolated antibody will be prepared by at least one purification step.

“Antibody-dependent cell-mediated cytotoxicity” or “ADCC” refers to a form of cytotoxicity in which secreted Ig bound onto Fc gamma receptors (FcγRs) present on certain cytotoxic cells (e.g. NK cells, neutrophils, and macrophages) enable these cytotoxic effector cells to bind specifically to an antigen-bearing target cell and subsequently kill the target cell e.g. with cytotoxins. To assess ADCC activity of an antibody of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells.

“Complement dependent cytotoxicity” or “CDC” refers to the lysis of a target cell in the presence of complement. Activation of the classical complement pathway is initiated by the binding of the first component of the complement system (Clq) to antibodies (of the appropriate subclass), which are bound to their cognate antigen. To assess complement activation, a CDC assay, e.g., as described in Gazzano-Santoro et al., J. Immunol. Methods 202: 163 (1996), may be performed. Polypeptide variants with altered Fc region amino acid sequences (polypeptides with a variant Fc region) and increased or decreased Clq binding are described, e.g., in U.S. Pat. No. 6,194,551 B1 and WO 1999/51642.

The term immunoconjugate (interchangeably referred to as “antibody-drug conjugate,” or “ADC”) refers to an antibody conjugated to one or more cytotoxic agents, such as a chemotherapeutic agent, a drug, a growth inhibitory agent, a toxin (e.g., a protein toxin, a enzymatically active toxin of bacterial, fungal, plant, or animal origin, or fragments thereof), or a radioactive isotope (i.e., a radioconjugate). Immunoconjugates have been used for the local delivery of cytotoxic agents, i.e., drugs that kill or inhibit the growth or proliferation of cells, in the treatment of cancer (e.g. Liu et al., Proc Natl. Acad. Sci. (1996), 93, 8618-8623)). Immunoconjugates allow for the targeted delivery of a drug moiety to a tumor, and intracellular accumulation therein, where systemic administration of unconjugated drugs may result in unacceptable levels of toxicity to normal cells and/or tissues. Toxins used in antibody-toxin conjugates include bacterial toxins such as diphtheria toxin, plant toxins such as ricin, small molecule toxins such as geldanamycin. The toxins may exert their cytotoxic effects by mechanisms including tubulin binding, DNA binding, or topoisomerase inhibition.

“Percent (%) sequence identity” with respect to a reference polynucleotide or polypeptide sequence, respectively, is defined as the percentage of nucleic acid or amino acid residues, respectively, in a candidate sequence that are identical with the nucleic acid or amino acid residues, respectively, in the reference polynucleotide or polypeptide sequence, respectively, after aligning the sequences and introducing gaps, if necessary, to achieve the maximum percent sequence identity. Conservative substitutions are not considered as part of the sequence identity. Preferred are un-gapped alignments. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The term ‘maturated antibodies’ or ‘maturated antigen-binding fragments’ such as maturated Fab variants includes derivatives of an antibody or antibody fragment exhibiting stronger binding—i.e. binding with increased affinity—to a given antigen such as the extracellular domain of the FGFR2. Maturation is the process of identifying a small number of mutations within the six CDRs of an antibody or antibody fragment leading to this affinity increase. The maturation process is the combination of molecular biology methods for introduction of mutations into the antibody and screening for identifying the improved binders.

### Antibodies of the Invention

The present invention relates to methods to inhibit growth of FGFR2-positive cancer cells and the progression of neoplastic disease by providing anti-FGFR2 antibodies. Provided are binding proteins, antibodies, antigen-binding antibody fragments thereof, and variants of the antibodies and fragments that reduce the surface expression of FGFR2 after binding to FGFR2 in both, a cell overexpressing FGFR2 and a cell expressing mutated FGFR2. It is another embodiment of the invention to provide antibodies, or antigen-binding antibody fragments thereof, or variants thereof, which bind to a broad range of different FGFR2 expressing cell lines both in cells overexpressing FGFR2 as well as in cells expressing mutated FGFR2 including, but not limited to SNU16 (ATCC-CRL-5974) and MFM223 (ECACC-98050130) which overexpress FGFR2 and AN3-CA (DSMZ-ACC 267) and MFE-296 (ECACC-98031101) which express mutated FGFR2.

Toward these ends, it is an embodiment of the invention to provide isolated human, humanized or chimeric antibodies, or antigen binding antibody fragments thereof, that specifically bind to a FGFR2 epitope which is present in different forms of the mature human FGFR2 polypeptide (for example see SEQ ID NO:61 for FGFR2 alpha IIIb, and SEQ ID NO:62 for FGFR2 beta IIIb), which is presented by FGFR2 expressing cancer cell lines/cancer cells, and/or which is bound by these antibodies with high affinities. As used herein, different ‘forms’ of FGFR2 include, but are not restricted to, different isoforms, different splice variants, different glycoforms or FGFR2 polypeptides which undergo different translational and posttranslational modifications. The FGFR2 polypeptide is named ‘FGFR2’ herein.

It is another embodiment of the invention to provide antibodies, or antigen-binding antibody fragments thereof, or variants thereof that are safe for human administration.

It is another embodiment of the invention to provide antibodies, or antigen-binding antibody fragments thereof, or variants thereof, which bind to human FGFR2 and are cross-reactive to FGFR2 of another species including, but not limited to murine, rat, macaca mulatta, rabbit, pig and dog FGFR2. Preferably, said other species is a rodent, such as for example mouse or rat. Most preferably, the antibodies, or antigen-binding antibody fragments thereof, or variants thereof bind to human FGFR2 and are cross-reactive to murine FGFR2.

It is another embodiment of the invention to provide antibodies, or antigen-binding antibody fragments thereof, or variants thereof, which are internalized efficiently following binding to a FGFR2 expressing cell. An antibody of the invention might be co-administered with known medicaments, and in some instances the antibody might itself be modified. For example, an antibody could be conjugated to a cytotoxic agent, immunotoxin, toxophore or radioisotope to potentially further increase efficacy.

It is another embodiment of the invention to provide antibodies, or antigen-binding antibody fragments thereof, or variants thereof, which activate FGFR2 on the short term and after internalization lead to FGFR2 degradation thus resulting in a desensitization of different FGFR2-expressing cancer cells or tumor cells for FGF stimulus and finally inhibit tumor growth in vivo.

It is another embodiment of the invention to provide antibodies which constitute a tool for diagnosis of malignant or dysplastic conditions in which FGFR2 expression is elevated compared to normal tissue or where FGFR2 is shed from the cell surface and becoming detectable in serum. Provided are anti-FGFR2 antibodies conjugated to a detectable marker. Preferred markers are a radiolabel, an enzyme, a chromophore or a fluorescer.

In one aspect, the invention provides an isolated antibody or antigen-binding fragment thereof that contains an antigen-binding region that binds to cell surface expressed FGFR2 and reduce after binding to FGFR2 the cell surface expression of FGFR2 in both a cell overexpressing FGFR2 and a cell expressing mutated FGFR2. In one embodiment, the invention provides an isolated antibody or antigen-binding fragment thereof that contains an antigen-binding region that specifically binds to native, cell surface expressed FGFR2 and reduces after binding to FGFR2 the cell surface expression of FGFR2 in both a cell overexpressing FGFR2 and a cell expressing mutated FGFR2. In one embodiment, the isolated antibody or antigen-binding fragment that binds specifically to native, cell surface expressed FGFR2 and reduces after binding to FGFR2 the cell surface expression of FGFR2 in both at least two different cells overexpressing FGFR2 and at least two different cells expressing mutated FGFR2.

In a further embodiment the antibody or antigen-binding fragment thereof specifically binds to native, cell surface expressed FGFR2 and (i) reduces after binding to FGFR2 the cell surface expression of FGFR2 in both, a cell overexpressing FGFR2 and a cell expressing mutated FGFR2 and (ii) induces FGFR2 phosphorylation.

In a further embodiment the antibody or antigen-binding fragment thereof specifically binds to native, cell surface expressed FGFR2 and (i) reduces after binding to FGFR2 the cell surface expression of FGFR2 in both, a cell overexpressing FGFR2 and a cell expressing mutated FGFR2 and (ii) induces FGFR2 phosphorylation, wherein the antibody desensitizes a FGFR2 expressing cell for stimulation with FGF7. In a further embodiment the desensitization is the desensitization of a FGFR2 overexpressing cell.

In a further embodiment the antibody or antigen-binding fragment thereof specifically binds to native, cell surface expressed FGFR2 and (i) reduces after binding to FGFR2 the cell surface expression of FGFR2 in both, a cell overexpressing FGFR2 and a cell expressing mutated FGFR2 and (ii) induces internalization of FGFR2 resulting in FGFR2 degradation.

In a further embodiment the antibody or antigen-binding fragment thereof specifically binds to native, cell surface expressed FGFR2 and (i) reduces after binding to FGFR2 the cell surface expression of FGFR2 in both a cell overexpressing FGFR2 and a cell expressing mutated FGFR2 and (ii) reduces tumor-growth in xenograft tumor experiments.

In a further embodiment the antibody or antigen-binding fragment thereof is capable to reduce the FGFR2 cell surface expression in different cell lines including, but not limited to SNU16 (ATCC-CRL-5974) and MFM223 (ECACC-98050130) which overexpress FGFR2 and in cell lines AN3-CA (DSMZ-ACC 267) and MFE-296 (ECACC-98031101) which express mutated FGFR2.

In a further embodiment the antibody or antigen-binding fragment thereof is capable to reduce after binding to FGFR2 the FGFR2 cell surface expression in SNU16 (ATCC-CRL-5974) and MFM223 (ECACC-98050130) cells which overexpress FGFR2 and in the cell lines AN3-CA (DSMZ-ACC 267) and MFE-296 (ECACC-98031101) which express mutated FGFR2.

In a preferred embodiment the cell surface reduction is at least 10%, 15%, 20%, 25% or 30% compared to the FGFR2 cell surface expression of the non-treated or the control treated cell.

In a further preferred embodiment the cell surface reduction after 96 hours is at least 10%, 15%, 20%, 25% or 30% compared to the FGFR2 cell surface expression of the non-treated or the control treated cell.

In a further embodiment the antibody or antigen-binding fragment thereof binds specifically to the extracellular N-terminal epitope (¹RPSFSLVEDTTLEPE¹⁵) of FGFR2 (SEQ ID NO:63). Critical residues for binding of the antibody or antigen-binding fragment thereof within the N-terminal epitope (¹RPSFSLVEDTTLEPE¹⁵) of FGFR2 include, but are not limited to, Arg 1, Pro 2, Phe 4, Ser 5, Leu 6 and Glu 8.

In a further embodiment the binding of the antibody or antigen-binding fragment thereof of the invention to the extracellular N-terminal epitope (SEQ ID NO:63) is mediated by at least one epitope residue selected from the group of residues consisting of Arg 1, Pro 2, Phe 4, Ser 5, Leu 6, and Glu 8.

In a further embodiment the binding of the antibody or antigen-binding fragment thereof of the invention to the extracellular N-terminal epitope (SEQ ID NO:63) is reduced by substitution of at least one epitope residue selected from the group of residues consisting of Arg 1, Pro 2, Phe 4, Ser 5, Leu 6, and Glu 8 by the amino acid Alanine.

In a further embodiment the binding of the antibody or antigen-binding fragment thereof of the invention to the extracellular N-terminal epitope (SEQ ID NO:63) is mediated by at least one epitope residue selected from the group of residues consisting of Pro 2, Leu 6 and Glu 8.

In a further embodiment the binding of the antibody or antigen-binding fragment thereof of the invention to the extracellular N-terminal epitope (SEQ ID NO:63) is reduced by substitution of at least one epitope residue selected from the group of residues consisting of Pro 2, Leu 6 and Glu 8 by the amino acid Alanine.

In another embodiment the binding of the antibody or antigen-binding fragment thereof of the invention to the extracellular N-terminal epitope (SEQ ID NO:63) is mediated by at least one epitope residue selected from the group of residues consisting of Pro 2, Leu 6 and Glu 8 and the binding to the epitope is invariant to sequence alterations of position 5 of the epitope.

In a further embodiment the binding of the antibody or antigen-binding fragment thereof of the invention to the extracellular N-terminal epitope (SEQ ID NO:63) is reduced by substitution of at least one epitope residue selected from the group of residues consisting of Pro 2, Leu 6 and Glu 8 by the amino acid Alanine and the binding to the epitope is invariant to sequence alterations of position 5 of the epitope.

In a further embodiment the antibody or antigen-binding fragment thereof loses more than 50% of its ELISA signal by changing of at least one of the amino acid residues in the N-terminal epitope (¹RPSFSLVEDTTLEPE¹⁵) of FGFR2 into an Alanine, (i) said residue selected from the group Pro 2, Leu 6 and Glu 8, or (ii) said residue selected from the group Arg 1, Pro 2, Phe 4 and Ser 5.

In a further preferred embodiment, the isolated antibodies or antigen-binding fragments thereof lose more than 50% of their ELISA signal by changing of at least one of the amino acid residues within the N-terminal epitope (¹RPSFSLVEDTTLEPE¹⁵) of FGFR2 into an Alanine wherein said residue is selected from the groups including, but not limited to a) Pro 2, Leu 6 and Glu 8 or b) Arg 1, Pro 2, Phe 4 and Ser 5, as depicted in Table 7.

In a further embodiment the antibodies or antigen-binding fragments compete in binding to FGFR2 with at least one antibody selected from the group “M048-D01”, “M047-D08”, “M017-B02”, “M021-H02”, “M054-A05”, “M054-D03”, “TPP-1397”, “TPP-1398”, “TPP-1399”, “TPP-1400”, “TPP-1401”, “TPP-1402”, “TPP-1403”, “TPP-1406”, “TPP-1407”, “TPP-1408”, “TPP-1409”, “TPP-1410”, “TPP-1411”, “TPP-1412”, and “TPP-1415”

Throughout this document, reference is made to the following preferred antibodies of the invention as depicted in Table 9 and Table 10: “M017-B02”, “M021-H02”, “M047-D08”, “M048-D01”, “M054-A05”, “M054-D03”, “TPP-1397”, “TPP-1398”, “TPP-1399”, “TPP-1400”, “TPP-1401”, “TPP-1402”, “TPP-1403”, “TPP-1406”, “TPP-1407”, “TPP-1408”, “TPP-1409”, “TPP-1410”, “TPP-1411”, “TPP-1412”, and “TPP-1415”.

M017-B02 represents an antibody comprising a variable heavy chain region corresponding to SEQ ID NO: 3 (DNA)/SEQ ID NO: 1 (protein) and a variable light chain region corresponding to SEQ ID NO: 4 (DNA)/SEQ ID NO: 2 (protein).

M0214-102 represents an antibody comprising a variable heavy chain region corresponding to SEQ ID NO: 13 (DNA)/SEQ ID NO: 11 (protein) and a variable light chain region corresponding to SEQ ID NO: 14 (DNA)/SEQ ID NO: 12 (protein).

M047-D08 represents an antibody comprising a variable heavy chain region corresponding to SEQ ID NO: 23 (DNA)/SEQ ID NO: 21 (protein) and a variable light chain region corresponding to SEQ ID NO: 24 (DNA)/SEQ ID NO: 22 (protein).

M048-D01 represents an antibody comprising a variable heavy chain region corresponding to SEQ ID NO: 33 (DNA)/SEQ ID NO: 31 (protein) and a variable light chain region corresponding to SEQ ID NO: 34 (DNA)/SEQ ID NO: 32 (protein).

M054-D03 represents an antibody comprising a variable heavy chain region corresponding to SEQ ID NO: 43 (DNA)/SEQ ID NO: 41 (protein) and a variable light chain region corresponding to SEQ ID NO: 44 (DNA)/SEQ ID NO: 42 (protein).

M054-A05 represents an antibody comprising a variable heavy chain region corresponding to SEQ ID NO: 53 (DNA)/SEQ ID NO: 51 (protein) and a variable light chain region corresponding to SEQ ID NO: 54 (DNA)/SEQ ID NO: 52 (protein).

TPP-1397 represents an antibody comprising a variable heavy chain region corresponding to SEQ ID NO: 83 (protein) and a variable light chain region corresponding to SEQ ID NO: 84 (protein).

TPP-1398 represents an antibody comprising a variable heavy chain region corresponding to SEQ ID NO: 93 (protein) and a variable light chain region corresponding to SEQ ID NO: 94 (protein).

TPP-1399 represents an antibody comprising a variable heavy chain region corresponding to SEQ ID NO: 103 (protein) and a variable light chain region corresponding to SEQ ID NO: 104 (protein).

TPP-1400 represents an antibody comprising a variable heavy chain region corresponding to SEQ ID NO: 113 (protein) and a variable light chain region corresponding to SEQ ID NO: 114 (protein).

TPP-1401 represents an antibody comprising a variable heavy chain region corresponding to SEQ ID NO: 123 (protein) and a variable light chain region corresponding to SEQ ID NO: 124 (protein).

TPP-1402 represents an antibody comprising a variable heavy chain region corresponding to SEQ ID NO: 133 (protein) and a variable light chain region corresponding to SEQ ID NO: 134 (protein).

TPP-1403 represents an antibody comprising a variable heavy chain region corresponding to SEQ ID NO: 73 (protein) and a variable light chain region corresponding to SEQ ID NO: 74 (protein).

TPP-1406 represents an antibody comprising a variable heavy chain region corresponding to SEQ ID NO: 153 (protein) and a variable light chain region corresponding to SEQ ID NO: 154 (protein).

TPP-1407 represents an antibody comprising a variable heavy chain region corresponding to SEQ ID NO: 163 (protein) and a variable light chain region corresponding to SEQ ID NO: 164 (protein).

TPP-1408 represents an antibody comprising a variable heavy chain region corresponding to SEQ ID NO: 173 (protein) and a variable light chain region corresponding to SEQ ID NO: 174 (protein).

TPP-1409 represents an antibody comprising a variable heavy chain region corresponding to SEQ ID NO: 183 (protein) and a variable light chain region corresponding to SEQ ID NO: 184 (protein).

TPP-1410 represents an antibody comprising a variable heavy chain region corresponding to SEQ ID NO: 193 (protein) and a variable light chain region corresponding to SEQ ID NO: 194 (protein).

TPP-1411 represents an antibody comprising a variable heavy chain region corresponding to SEQ ID NO: 203 (protein) and a variable light chain region corresponding to SEQ ID NO: 204 (protein).

TPP-1412 represents an antibody comprising a variable heavy chain region corresponding to SEQ ID NO: 213 (protein) and a variable light chain region corresponding to SEQ ID NO: 214 (protein).

TPP-1415 represents an antibody comprising a variable heavy chain region corresponding to SEQ ID NO: 143 (protein) and a variable light chain region corresponding to SEQ ID NO: 144 (protein). In a further preferred embodiment the antibodies or antigen-binding fragments comprise heavy or light chain CDR sequences which are at least 50%, 55%, 60% 70%, 80%, 90, or 95% identical to at least one, preferably corresponding, CDR sequence of the antibodies “M048-D01”, “M047-D08”, “M017-B02”, “M0214-102”, “M054-A05”, “M054-D03”, “TPP-1397”, “TPP-1398”, “TPP-1399”, “TPP-1400”, “TPP-1401”, “TPP-1402”, “TPP-1403”, “TPP-1406”, “TPP-1407”, “TPP-1408”, “TPP-1409”, “TPP-1410”, “TPP-1411”, “TPP-1412” or “TPP-1415” or at least 50%, 60%, 70%, 80%, 90%, 92% or 95% identical to the VH or VL sequence of “M048-D01”, “M047-D08”, “M017-B02”, “M021-H02”, “M054-A05”, “M054-D03”, “TPP-1397”, “TPP-1398”, “TPP-1399”, “TPP-1400”, “TPP-1401”, “TPP-1402”, “TPP-1403”, “TPP-1406”, “TPP-1407”, “TPP-1408”, “TPP-1409”, “TPP-1410”, “TPP-1411”, “TPP-1412” or “TPP-1415”, respectively.

In a further preferred embodiment the antibody or antigen-binding fragment of the invention comprises at least one CDR sequence or at least one variable heavy chain or light chain sequence as depicted in Table 9 and Table 10.

In a more preferred embodiment the antibody of the invention or antigen-binding fragment thereof comprises a heavy chain antigen-binding region that comprises SEQ ID NO:5 (H-CDR1), SEQ ID NO:6 (H-CDR2) and SEQ ID NO:7 (H-CDR3) and comprises a light chain antigen-binding region that comprises SEQ ID NO:8 (L-CDR1), SEQ ID NO:9 (L-CDR2) and SEQ ID NO:10 (L-CDR3).

In a more preferred embodiment the antibody of the invention or antigen-binding fragment thereof comprises a heavy chain antigen-binding region that comprises SEQ ID NO:15 (H-CDR1), SEQ ID NO:16 (H-CDR2) and SEQ ID NO:17 (H-CDR3) and comprises a light chain antigen-binding region that comprises SEQ ID NO:18 (L-CDR1), SEQ ID NO:19 (L-CDR2) and SEQ ID NO:20 (L-CDR3).

In a more preferred embodiment the antibody of the invention or antigen-binding fragment thereof comprises a heavy chain antigen-binding region that comprises SEQ ID NO:25 (H-CDR1), SEQ ID NO:26 (H-CDR2) and SEQ ID NO:27 (H-CDR3) and comprises a light chain antigen-binding region that comprises SEQ ID NO:28 (L-CDR1), SEQ ID NO:29 (L-CDR2) and SEQ ID NO:30 (L-CDR3).

In a more preferred embodiment the antibody of the invention or antigen-binding fragment thereof comprises a heavy chain antigen-binding region that comprises SEQ ID NO:35 (H-CDR1), SEQ ID NO:36 (H-CDR2) and SEQ ID NO:37 (H-CDR3) and comprises a light chain antigen-binding region that comprises SEQ ID NO:38 (L-CDR1), SEQ ID NO:39 (L-CDR2) and SEQ ID NO:40 (L-CDR3).

In a more preferred embodiment the antibody of the invention or antigen-binding fragment thereof comprises a heavy chain antigen-binding region that comprises SEQ ID NO:45 (H-CDR1), SEQ ID NO:46 (H-CDR2) and SEQ ID NO:47 (H-CDR3) and comprises a light chain antigen-binding region that comprises SEQ ID NO:48 (L-CDR1), SEQ ID NO:49 (L-CDR2) and SEQ ID NO:50 (L-CDR3).

In a more preferred embodiment the antibody of the invention or antigen-binding fragment thereof comprises a heavy chain antigen-binding region that comprises SEQ ID NO:55 (H-CDR1), SEQ ID NO:56 (H-CDR2) and SEQ ID NO:57 (H-CDR3) and comprises a light chain antigen-binding region that comprises SEQ ID NO:58 (L-CDR1), SEQ ID NO:59 (L-CDR2) and SEQ ID NO:60 (L-CDR3).

In a more preferred embodiment the antibody of the invention or antigen-binding fragment thereof comprises a heavy chain antigen-binding region that comprises SEQ ID NO:75 (H-CDR1), SEQ ID NO:76 (H-CDR2) and SEQ ID NO:77 (H-CDR3) and comprises a light chain antigen-binding region that comprises SEQ ID NO:78 (L-CDR1), SEQ ID NO:79 (L-CDR2) and SEQ ID NO:80 (L-CDR3).

In a more preferred embodiment the antibody of the invention or antigen-binding fragment thereof comprises a heavy chain antigen-binding region that comprises SEQ ID NO:85 (H-CDR1), SEQ ID NO:86 (H-CDR2) and SEQ ID NO:87 (H-CDR3) and comprises a light chain antigen-binding region that comprises SEQ ID NO:88 (L-CDR1), SEQ ID NO:89 (L-CDR2) and SEQ ID NO:90 (L-CDR3).

In a more preferred embodiment the antibody of the invention or antigen-binding fragment thereof comprises a heavy chain antigen-binding region that comprises SEQ ID NO:95 (H-CDR1), SEQ ID NO:96 (H-CDR2) and SEQ ID NO:97 (H-CDR3) and comprises a light chain antigen-binding region that comprises SEQ ID NO:98 (L-CDR1), SEQ ID NO:99 (L-CDR2) and SEQ ID NO:100 (L-CDR3).

In a more preferred embodiment the antibody of the invention or antigen-binding fragment thereof comprises a heavy chain antigen-binding region that comprises SEQ ID NO:105 (H-CDR1), SEQ ID NO:106 (H-CDR2) and SEQ ID NO:107 (H-CDR3) and comprises a light chain antigen-binding region that comprises SEQ ID NO:108 (L-CDR1), SEQ ID NO:109 (L-CDR2) and SEQ ID NO:110 (L-CDR3).

In a more preferred embodiment the antibody of the invention or antigen-binding fragment thereof comprises a heavy chain antigen-binding region that comprises SEQ ID NO:115 (H-CDR1), SEQ ID NO:116 (H-CDR2) and SEQ ID NO:117 (H-CDR3) and comprises a light chain antigen-binding region that comprises SEQ ID NO:118 (L-CDR1), SEQ ID NO:119 (L-CDR2) and SEQ ID NO:120 (L-CDR3).

In a more preferred embodiment the antibody of the invention or antigen-binding fragment thereof comprises a heavy chain antigen-binding region that comprises SEQ ID NO:125 (H-CDR1), SEQ ID NO:126 (H-CDR2) and SEQ ID NO:127 (H-CDR3) and comprises a light chain antigen-binding region that comprises SEQ ID NO:128 (L-CDR1), SEQ ID NO:129 (L-CDR2) and SEQ ID NO:130 (L-CDR3).

In a more preferred embodiment the antibody of the invention or antigen-binding fragment thereof comprises a heavy chain antigen-binding region that comprises SEQ ID NO:135 (H-CDR1), SEQ ID NO:136 (H-CDR2) and SEQ ID NO:137 (H-CDR3) and comprises a light chain antigen-binding region that comprises SEQ ID NO:138 (L-CDR1), SEQ ID NO:139 (L-CDR2) and SEQ ID NO:140 (L-CDR3).

In a more preferred embodiment the antibody of the invention or antigen-binding fragment thereof comprises a heavy chain antigen-binding region that comprises SEQ ID NO:145 (H-CDR1), SEQ ID NO:146 (H-CDR2) and SEQ ID NO:147 (H-CDR3) and comprises a light chain antigen-binding region that comprises SEQ ID NO:148 (L-CDR1), SEQ ID NO:149 (L-CDR2) and SEQ ID NO:150 (L-CDR3).

In a more preferred embodiment the antibody of the invention or antigen-binding fragment thereof comprises a heavy chain antigen-binding region that comprises SEQ ID NO:155 (H-CDR1), SEQ ID NO:156 (H-CDR2) and SEQ ID NO:157 (H-CDR3) and comprises a light chain antigen-binding region that comprises SEQ ID NO:158 (L-CDR1), SEQ ID NO:159 (L-CDR2) and SEQ ID NO:160 (L-CDR3).

In a more preferred embodiment the antibody of the invention or antigen-binding fragment thereof comprises a heavy chain antigen-binding region that comprises SEQ ID NO:165 (H-CDR1), SEQ ID NO:166 (H-CDR2) and SEQ ID NO:167 (H-CDR3) and comprises a light chain antigen-binding region that comprises SEQ ID NO:168 (L-CDR1), SEQ ID NO:169 (L-CDR2) and SEQ ID NO:170 (L-CDR3).

In a more preferred embodiment the antibody of the invention or antigen-binding fragment thereof comprises a heavy chain antigen-binding region that comprises SEQ ID NO:175 (H-CDR1), SEQ ID NO:176 (H-CDR2) and SEQ ID NO:177 (H-CDR3) and comprises a light chain antigen-binding region that comprises SEQ ID NO:178 (L-CDR1), SEQ ID NO:179 (L-CDR2) and SEQ ID NO:180 (L-CDR3).

In a more preferred embodiment the antibody of the invention or antigen-binding fragment thereof comprises a heavy chain antigen-binding region that comprises SEQ ID NO:185 (H-CDR1), SEQ ID NO:186 (H-CDR2) and SEQ ID NO:187 (H-CDR3) and comprises a light chain antigen-binding region that comprises SEQ ID NO:188 (L-CDR1), SEQ ID NO:189 (L-CDR2) and SEQ ID NO:190 (L-CDR3).

In a more preferred embodiment the antibody of the invention or antigen-binding fragment thereof comprises a heavy chain antigen-binding region that comprises SEQ ID NO:195 (H-CDR1), SEQ ID NO:196 (H-CDR2) and SEQ ID NO:197 (H-CDR3) and comprises a light chain antigen-binding region that comprises SEQ ID NO:198 (L-CDR1), SEQ ID NO:199 (L-CDR2) and SEQ ID NO:200 (L-CDR3).

In a more preferred embodiment the antibody of the invention or antigen-binding fragment thereof comprises a heavy chain antigen-binding region that comprises SEQ ID NO:205 (H-CDR1), SEQ ID NO:206 (H-CDR2) and SEQ ID NO:207 (H-CDR3) and comprises a light chain antigen-binding region that comprises SEQ ID NO:208 (L-CDR1), SEQ ID NO:209 (L-CDR2) and SEQ ID NO:210 (L-CDR3).

In a more preferred embodiment the antibody of the invention or antigen-binding fragment thereof comprises a heavy chain antigen-binding region that comprises SEQ ID NO:215 (H-CDR1), SEQ ID NO:216 (H-CDR2) and SEQ ID NO:217 (H-CDR3) and comprises a light chain antigen-binding region that comprises SEQ ID NO:218 (L-CDR1), SEQ ID NO:219 (L-CDR2) and SEQ ID NO:220 (L-CDR3).

An antibody of the invention may be an IgG (e.g., IgG1 IgG2, IgG3, IgG4), while an antibody fragment may be a Fab, Fab′, F(ab′)2 or scFv, for example. An inventive antibody fragment, accordingly, may be, or may contain, an antigen-binding region that behaves in one or more ways as described herein.

For example the antibody Fab fragment M048-D01 (SEQ ID NO:31 for VH chain, and SEQ ID NO:32 for VL chain) was expressed as human IgG1 M048-D01-hIgG1 (SEQ ID NO:67 for heavy chain, and SEQ ID NO:68 for light chain) and Fab fragment M047-D08 (SEQ ID NO:21 for VH chain, and SEQ ID NO:22 for VL chain) was expressed as human IgG1 M047-D08-hIgG1 (SEQ ID NO:69 for heavy chain, and SEQ ID NO:70 for light chain). For efficient cloning the first 3 amino acids of the N-terminus of the heavy chains [EVQ] (SEQ ID NO:67 and SEQ ID NO:69) can also alternatively be expressed as [QVE], for example as a variant of the heavy chain of human IgG1 M048-D01-hIgG1 (SEQ ID NO:222). For efficient cloning the N-terminus of light chains can be extended by amino acid residues e.g. Alanin.

In a preferred embodiment the antibodies or antigen-binding antibody fragments of the invention are monoclonal. In a further preferred embodiment the antibodies or antigen-binding antibody fragments of the invention are human, humanized or chimeric.

In another aspect, the invention provides antibodies or antigen-binding fragments having an antigen-binding region that bind specifically to and/or has a high affinity for FGFR2 independent of alpha and beta isoforms as well as IIIb and IIIc splice forms (for example see SEQ ID NO:61 for FGFR2 alpha IIIb and SEQ ID NO:62 for FGFR2 beta IIIb). An antibody or antigen-binding fragment is said to have a “high affinity” for an antigen if the affinity measurement is less than 250 nM (monovalent affinity of the antibody or antigen-binding fragment). An inventive antibody or antigen-binding region preferably can bind to human FGFR2 with an affinity of less than 250 nM, preferably less than 150 nM, determined as monovalent affinity to human FGFR2. For instance, the affinity of an antibody of the invention against FGFR2 from different species may be around 100 nM (monovalent affinity of the antibody or antigen-binding fragment) as shown in Table 8 exemplarily for M048-D1 and M047-D08.

The IgG1 format was used for the cell-based affinity determination by fluorescence-activated cell sorting (FACS). Table 6 provides a summary of the binding strength (EC50) of representative anti-FGFR2-IgG antibodies on cancer cell lines of human (SNU16, MFM223), murine (4T1) and rat (RUCA) origin.

An IgG 1 is said to have a “high affinity” for an antigen if the affinity measurement measured by FACS is less than 100 nM (apparent affinity of IgG). An inventive bivalent antibody or antigen-binding fragment preferably can bind to FGFR2 with an affinity of less than 100 nM, more preferably less than 50 nM, and still more preferably less than 10 nM. Further preferred are bivalent antibodies that bind to FGFR2 with an affinity of less than 5 nM, and more preferably less than 1 nM determined as apparent affinity of an IgG to FGFR2. For instance, the apparent affinity of an antibody of the invention against FGFR2 may be about 89.5 nM or less than 0.1 nM on different tumor cell lines of human, murine and rat origin as determined by FACS analysis as depicted in Table 6.

An antibody or antigen-binding fragment of the invention internalizes “efficiently” when its time of half maximal internalization (t ½) into FGFR2 expressing tumor cells is shorter than 180 min or more preferably shorter than 120 min and still more preferably shorter than 90 min. Further preferred are antibodies or antigen-binding fragments with half maximal internalization times (t ½) of 60 minutes or less as determined by the protocol described in example 12.

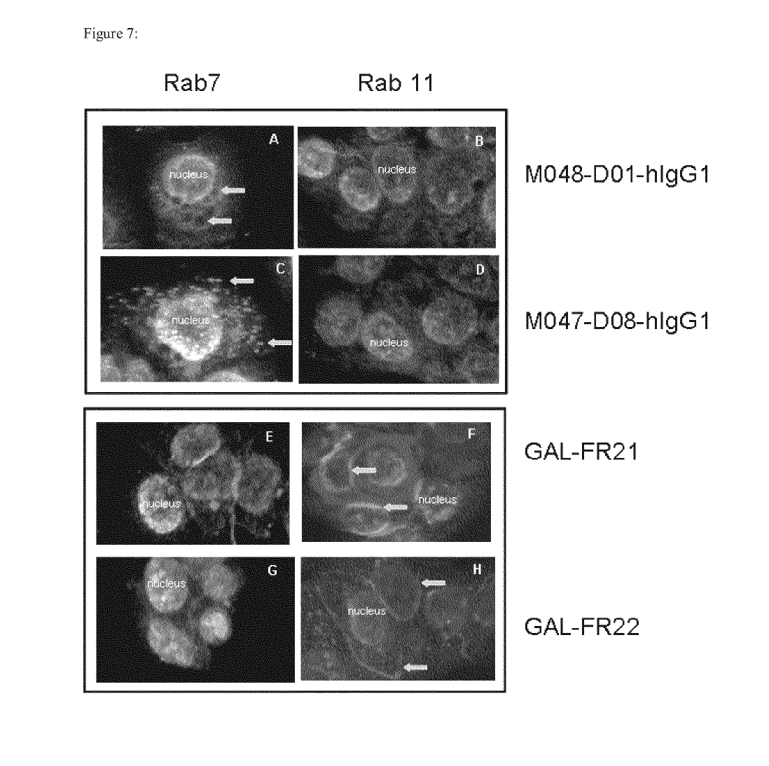

Co-staining of small G-proteins can be used for a more detailed evaluation of the trafficking pathway of antibodies after internalization. For instance Rab GTPases which regulate many steps of membrane traffic, including vesicle formation, vesicle movement along actin and tubulin networks, and membrane fusion can be used to distinguish between different pathways. Thereby, co-staining of labeled antibodies with Rab7, which is expressed in late endosomes and lysosomes, indicates that after internalization of FGFR2 the complex enters the endosomal-lysosomal pathway, whereas co-staining with Rab11, which is expressed in early and recycling endosomes, indicates that these antibodies internalize after binding to FGFR2 and favor the recycling pathway. Entering the endosomal-lysosomal pathway enables the antibodies to induce degradation of FGFR2 after internalization which finally results in desensitization of this pathway. FIG. 7 shows the co-staining patterns of representative antibodies of the invention with Rab7 and Rab11 as described in example 12.

Internalizable antibodies or antigen-binding fragments of the invention are suitable as targeting moiety of an antibody-drug conjugate (ADC). An antibody or antigen-binding fragment is suitable in an in vitro or in vivo method to deliver a compound, preferably a cytotoxic agent, into a FGFR2 expressing cell.

In some embodiments, the antibody, antigen-binding fragment thereof, or derivative thereof or nucleic acid encoding the same is isolated. An isolated biological component (such as a nucleic acid molecule or protein such as an antibody) is one that has been substantially separated or purified away from other biological components in the cell of the organism in which the component naturally occurs, e.g., other chromosomal and extra-chromosomal DNA and RNA, proteins and organelles. Nucleic acids and proteins that have been “isolated” include nucleic acids and proteins purified by standard purification methods as described for example in Sambrook et al., 1989 (Sambrook, J., Fritsch, E. F. and Maniatis, T. (1989) Molecular Cloning: A laboratory manual, Cold Spring Harbor Laboratory Press, Cold Spring Harbor, USA) and Robert K. Scopes et al. 1994 (Protein Purification, —Principles and Practice, Springer Science and Business Media LLC). The term also embraces nucleic acids and proteins prepared by recombinant expression in a host cell as well as chemically synthesized nucleic acids.

An antibody of the invention may be derived from a recombinant antibody library that is based on amino acid sequences that have been isolated from the antibodies of a large number of healthy volunteers. Using the n-CoDeR® technology the fully human CDRs are recombined into new antibody molecules. The unique recombination process allows the library to contain a wider variety of antibodies than could have been created naturally by the human immune system.

### Antibody Generation

A fully human N-CoDeR antibody phage display library was used to isolate FGFR2-specific, human monoclonal antibodies of the present invention by a combination of whole cell and protein panning and through the development of specific methods. These methods include the development of panning procedures and screening assays capable of identifying antibodies that preferentially bind to FGFR2 displayed on the cell surface and that are cross-reactive to murine FGFR2 and FGFR2 from other species and have a binding and functional activity which is independent of FGFR2 over-expression and common mutations of FGFR2 found in FGFR2-related diseases such as, cancer.

Antibodies to the cell-surface FGFR2 were developed by a combination of three non-conventional approaches in phage-display technology (PDT). First, selections were performed with recombinant, soluble, human and murine FGFR2 Fc-fusion proteins of several splice variants (alpha, beta, IIIb and IIIc) to select for a very broad splice variant cross-reactivity. Second, in addition cell-surface selections were performed with KATO III cells expressing FGFR2 on their cell-surface. Third, screening methods were developed which allowed for successive screening of the phage outputs obtained in panning on whole KATOIII cells and recombinant, soluble, human and murine FGFR1, FGFR2, FGFR3, and FGFR4 Fc fusion proteins of several splice variants (alpha, beta, IIIb and IIIc) to select for FGFR2 specific binders (no binding to FGFR1, FGFR3, and FGFR4) with a very broad splice variant cross-reactivity.

After identification of preferred Fab fragments these were expressed as full length IgGs. For example the antibody Fab fragment M048-D01 (SEQ ID NO:31 for VH chain, and SEQ ID NO:32 for VL chain) was expressed as human IgG1 M048-D01-hIgG1 (SEQ ID NO:67 for heavy chain, and SEQ ID NO:68 for light chain) and Fab fragment M047-D08 (SEQ ID NO:21 for VH chain, and SEQ ID NO:22 for VL chain) was expressed as human IgG1 M047-D08-hIgG1 (SEQ ID NO:69 for heavy chain, and SEQ ID NO:70 for light chain). For efficient cloning the first 3 amino acids of the N-terminus of the heavy chains [EVQ] (SEQ ID NO:67 and SEQ ID NO:69) can also alternatively be expressed as [QVE], for example as a variant of the heavy chain of human IgG1 M048-D01-hIgG1 (SEQ ID NO:222). For efficient cloning the N-terminus of light chains can be extended by amino acid residues e.g. Alanin Theses constructs were for example transiently expressed in mammalian cells as described in Tom et al., Chapter 12 in Methods Express: Expression Systems edited by Micheal R. Dyson and Yves Durocher, Scion Publishing Ltd, 2007. Briefly, a CMV-Promoter based expression plasmid was transfected into HEK293-6E cells and incubated in Fernbach—Flasks or Wave-Bags. Expression was at 37° C. for 5 to 6 days in F17 Medium (Invitrogen). 5 g/l Tryptone TN1 (Organotechnie), 1% Ultra-Low IgG FCS (Invitrogen) and 0.5 mM Valproic acid (Sigma) were supplemented 24 h post-transfection.

These antibodies were further characterized by their binding affinity in ELISA's, and by BIAcore binding to soluble FGFR2. FACS binding with cells from different species was performed to select for cell binding antibodies which have a high affinity on mouse, rat and human cancer cell lines.

The combination of these specific methods allowed the isolation of the unique antibodies “M017-B02”, “M021-H02”, “M047-D08”, “M048-D01”, “M054-A05” and, “M054-D03”.

Further characterization revealed that the selected antibodies bind to a unique epitope at the N-terminus of FGFR2 resulting in their special features. These unique antibodies were further characterized in in vitro phosphorylation assays, internalization assays, and in vivo tumor xenograft experiments. The selected antibodies show a strong and significant anti-tumor activity in tumor xenograft experiments with SNU16 cells.

### Peptide Variants

Antibodies or antigen-binding fragments of the invention are not limited to the specific peptide sequences provided herein. Rather, the invention also embodies variants of these polypeptides. With reference to the instant disclosure and conventionally available technologies and references, the skilled worker will be able to prepare, test and utilize functional variants of the antibodies disclosed herein, while appreciating these variants having the ability to bind to FGFR2 fall within the scope of the present invention.

A variant can include, for example, an antibody that has at least one altered complementary determining region (CDR) (hyper-variable) and/or framework (FR) (variable) domain/position, vis-à-vis a peptide sequence disclosed herein. To better illustrate this concept, a brief description of antibody structure follows.

An antibody is composed of two peptide chains, each containing one (light chain) or three (heavy chain) constant domains and a variable region (VL, VH), the latter of which is in each case made up of four FR regions and three interspaced CDRs. The antigen-binding site is formed by one or more CDRs, yet the FR regions provide the structural framework for the CDRs and, hence, play an important role in antigen binding. By altering one or more amino acid residues in a CDR or FR region, the skilled worker routinely can generate mutated or diversified antibody sequences, which can be screened against the antigen, for new or improved properties, for example.

A further preferred embodiment of the invention is an antibody or antigen-binding fragment in which the VH and VL sequences are selected as shown in Table 9. The skilled worker can use the data in Table 9 to design peptide variants that are within the scope of the present invention. It is preferred that variants are constructed by changing amino acids within one or more CDR regions; a variant might also have one or more altered framework regions. Alterations also may be made in the framework regions. For example, a peptide FR domain might be altered where there is a deviation in a residue compared to a germline sequence.

Alternatively, the skilled worker could make the same analysis by comparing the amino acid sequences disclosed herein to known sequences of the same class of such antibodies, using, for example, the procedure described by Knappik A., et al., JMB 2000, 296:57-86.

Furthermore, variants may be obtained by using one antibody as starting point for optimization by diversifying one or more amino acid residues in the antibody, preferably amino acid residues in one or more CDRs, and by screening the resulting collection of antibody variants for variants with improved properties. Particularly preferred is diversification of one or more amino acid residues in CDR3 of VL and/or VH, Diversification can be done by synthesizing a collection of DNA molecules using trinucleotide mutagenesis (TRIM) technology (Virnekäs B. et al., Nucl. Acids Res. 1994, 22: 5600.). Antibodies or antigen-binding fragments thereof include molecules with modifications/variations including but not limited to e.g. modifications leading to altered half-life (e.g. modification of the Fc part or attachment of further molecules such as PEG), altered binding affinity or altered ADCC or CDC activity.

Examples of variants of antibodies are given for M048-D01 (TPP-1397, TPP-1398, TPP-1399, TPP-1400, TPP-1401, TPP-1402 and TPP-1403) and M047-D08 (TPP-1406, TPP-1407, TPP-1408, TPP-1409, TPP-1410, TPP-1411, TPP-1412, and TPP-1415) as depicted in Table 10. The improved properties of these variant antibodies are shown in Table 11.

### Conservative Amino Acid Variants

Polypeptide variants may be made that conserve the overall molecular structure of an antibody peptide sequence described herein. Given the properties of the individual amino acids, some rational substitutions will be recognized by the skilled worker. Amino acid substitutions, i.e., “conservative substitutions,” may be made, for instance, on the basis of similarity in polarity, charge, solubility, hydrophobicity, hydrophilicity, and/or the amphipathic nature of the residues involved.

For example, (a) nonpolar (hydrophobic) amino acids include alanine, leucine, isoleucine, valine, proline, phenylalanine, tryptophane, and methionine; (b) polar neutral amino acids include glycine, serine, threonine, cysteine, tyrosine, asparagine, and glutamine; (c) positively charged (basic) amino acids include arginine, lysine, and histidine; and (d) negatively charged (acidic) amino acids include aspartic acid and glutamic acid. Substitutions typically may be made within groups (a)-(d). In addition, glycine and proline may be substituted for one another based on their ability to disrupt α-helices. Similarly, certain amino acids, such as alanine, cysteine, leucine, methionine, glutamic acid, glutamine, histidine and lysine are more commonly found in α-helices, while valine, isoleucine, phenylalanine, tyrosine, tryptophan and threonine are more commonly found in β-pleated sheets. Glycine, serine, aspartic acid, asparagine, and proline are commonly found in turns. Some preferred substitutions may be made among the following groups: (i) S and T; (ii) P and G; and (iii) A, V, L and I. Given the known genetic code, and recombinant and synthetic DNA techniques, the skilled scientist readily can construct DNAs encoding the conservative amino acid variants.

As used herein, “sequence identity” between two polypeptide sequences, indicates the percentage of amino acids that are identical between the sequences. “Sequence homology” indicates the percentage of amino acids that either is identical or that represent conservative amino acid substitutions.

### DNA Molecules of the Invention

The present invention also relates to the DNA molecules that encode an antibody of the invention or antigen-binding fragment thereof. These sequences include, but are not limited to, those DNA molecules set forth in SEQ IDs 3, 4, 13, 14, 23, 24, 33, 34, 43, 44, 53 and 54.

DNA molecules of the invention are not limited to the sequences disclosed herein, but also include variants thereof. DNA variants within the invention may be described by reference to their physical properties in hybridization. The skilled worker will recognize that DNA can be used to identify its complement and, since DNA is double stranded, its equivalent or homolog, using nucleic acid hybridization techniques. It also will be recognized that hybridization can occur with less than 100% complementarity. However, given appropriate choice of conditions, hybridization techniques can be used to differentiate among DNA sequences based on their structural relatedness to a particular probe. For guidance regarding such conditions see, Sambrook et al., 1989 supra and Ausubel et al., 1995 (Ausubel, F. M., Brent, R., Kingston, R. E., Moore, D. D., Sedman, J. G., Smith, J. A., & Struhl, K. eds. (1995). Current Protocols in Molecular Biology. New York: John Wiley and Sons).

Structural similarity between two polynucleotide sequences can be expressed as a function of “stringency” of the conditions under which the two sequences will hybridize with one another. As used herein, the term “stringency” refers to the extent that the conditions disfavor hybridization. Stringent conditions strongly disfavor hybridization, and only the most structurally related molecules will hybridize to one another under such conditions. Conversely, non-stringent conditions favor hybridization of molecules displaying a lesser degree of structural relatedness. Hybridization stringency, therefore, directly correlates with the structural relationships of two nucleic acid sequences. The following relationships are useful in correlating hybridization and relatedness (where Tₘ is the melting temperature of a nucleic acid duplex):
a. Tₘ=69.3+0.41(G+C) %
b. The Tₘ of a duplex DNA decreases by 1° C. with every increase of 1% in the number of mismatched base pairs.
c. (Tₘ)_{μ2}−(Tₘ)_{μ1}=18.5 log₁₀μ2/μl
   where μ1 and μ2 are the ionic strengths of two solutions.

Hybridization stringency is a function of many factors, including overall DNA concentration, ionic strength, temperature, probe size and the presence of agents which disrupt hydrogen bonding. Factors promoting hybridization include high DNA concentrations, high ionic strengths, low temperatures, longer probe size and the absence of agents that disrupt hydrogen bonding. Hybridization typically is performed in two phases: the “binding” phase and the “washing” phase.

### Functionally Equivalent Variants

Yet another class of DNA variants within the scope of the invention may be described with reference to the product they encode. These functionally equivalent polynucleotides are characterized by the fact that they encode the same peptide sequences found in SEQ ID NOS: 1, 2, 5-12, 15-22, 25-32, 35-42, 45-52, 55-60 due to the degeneracy of the genetic code.

It is recognized that variants of DNA molecules provided herein can be constructed in several different ways. For example, they may be constructed as completely synthetic DNAs. Methods of efficiently synthesizing oligonucleotides in the range of 20 to about 150 nucleotides are widely available. See Ausubel et al., section 2.11, Supplement 21 (1993). Overlapping oligonucleotides may be synthesized and assembled in a fashion first reported by Khorana et al., J. Mol. Biol. 72:209-217 (1971); see also Ausubel et al., supra, Section 8.2. Synthetic DNAs preferably are designed with convenient restriction sites engineered at the 5′ and 3′ ends of the gene to facilitate cloning into an appropriate vector.

As indicated, a method of generating variants is to start with one of the DNAs disclosed herein and then to conduct site-directed mutagenesis. See Ausubel et al., supra, chapter 8, Supplement 37 (1997). In a typical method, a target DNA is cloned into a single-stranded DNA bacteriophage vehicle, Single-stranded DNA is isolated and hybridized with an oligonucleotide containing the desired nucleotide alteration(s). The complementary strand is synthesized and the double stranded phage is introduced into a host. Some of the resulting progeny will contain the desired mutant, which can be confirmed using DNA sequencing. In addition, various methods are available that increase the probability that the progeny phage will be the desired mutant. These methods are well known to those in the field and kits are commercially available for generating such mutants.

### Recombinant DNA Constructs and Expression

The present invention further provides recombinant DNA constructs comprising one or more of the nucleotide sequences of the present invention. The recombinant constructs of the present invention are used in connection with a vector, such as a plasmid, phagemid, phage or viral vector, into which a DNA molecule encoding an antibody of the invention or antigen-binding fragment thereof is inserted.

An antibody, antigen binding portion, or derivative thereof provided herein can be prepared by recombinant expression of nucleic acid sequences encoding light and heavy chains or portions thereof in a host cell. To express an antibody, antigen binding portion, or derivative thereof recombinantly, a host cell can be transfected with one or more recombinant expression vectors carrying DNA fragments encoding the light and/or heavy chains or portions thereof such that the light and heavy chains are expressed in the host cell. Standard recombinant DNA methodologies are used prepare and/or obtain nucleic acids encoding the heavy and light chains, incorporate these nucleic acids into recombinant expression vectors and introduce the vectors into host cells, such as those described in Sambrook, Fritsch and Maniatis (eds.), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

In addition, the nucleic acid sequences encoding variable regions of the heavy and/or light chains can be converted, for example, to nucleic acid sequences encoding full-length antibody chains, Fab fragments, or to scFv. The VL- or VH-encoding DNA fragment can be operatively linked, (such that the amino acid sequences encoded by the two DNA fragments are in-frame) to another DNA fragment encoding, for example, an antibody constant region or a flexible linker. The sequences of human heavy chain and light chain constant regions are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification.

In certain assays an expression of the antibodies of this invention as murine IgG is preferred, e.g. immunohistochemistry with human samples can be analyzed more easily by using murine antibodies. Therefore, for example the antibody Fab fragment M048-D01 (SEQ ID NO:31 for VH chain, and SEQ ID NO:32 for VL chain) was expressed as murine IgG2a called M048-D01-mIgG2a (SEQ ID NO:221 for heavy chain). This antibody was also used in Example 17 as control.

To create a polynucleotide sequence that encodes a scFv, the VH- and VL-encoding nucleic acids can be operatively linked to another fragment encoding a flexible linker such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, antigen binding portions or derivatives thereof standard recombinant DNA expression methods can be used (see, for example, Goeddel; Gene Expression Technology. Methods in Enzymology 185, Academic Press, San Diego, Calif. (1990)). For example, DNA encoding the desired polypeptide can be inserted into an expression vector which is then transfected into a suitable host cell. Suitable host cells are prokaryotic and eukaryotic cells. Examples for prokaryotic host cells are e.g. bacteria, examples for eukaryotic host cells are yeast, insect or mammalian cells. In some embodiments, the DNAs encoding the heavy and light chains are inserted into separate vectors. In other embodiments, the DNA encoding the heavy and light chains is inserted into the same vector. It is understood that the design of the expression vector, including the selection of regulatory sequences is affected by factors such as the choice of the host cell, the level of expression of protein desired and whether expression is constitutive or inducible.

### Bacterial Expression

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include E. coli, Bacillus subtilis, Salmonella typhimurium and various species within the genera Pseudomonas, Streptomyces, and Staphylococcus.

Bacterial vectors may be, for example, bacteriophage-, plasmid- or phagemid-based. These vectors can contain a selectable marker and bacterial origin of replication derived from commercially available plasmids typically containing elements of the well-known cloning vector pBR322 (ATCC 37017). Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification.

In bacterial systems, a number of expression vectors may be advantageously selected depending upon the use intended for the protein being expressed. For example, when a large quantity of such a protein is to be produced, for the generation of antibodies or to screen peptide libraries, for example, vectors which direct the expression of high levels of fusion protein products that are readily purified may be desirable.

Therefore an embodiment of the present invention is an expression vector comprising a nucleic acid sequence encoding for the novel antibodies of the present invention. See Example 2 for an exemplary description.

Antibodies of the present invention or antigen-binding fragment thereof include naturally purified products, products of chemical synthetic procedures, and products produced by recombinant techniques from a prokaryotic host, including, for example, E. coli, Bacillus subtilis, Salmonella typhimurium and various species within the genera Pseudomonas, Streptomyces, and Staphylococcus, preferably, from E. coli cells.

### Mammalian Expression & Purification

Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al. The recombinant expression vectors can also include origins of replication and selectable markers (see e.g., U.S. Pat. Nos. 4,399,216, 4,634,665 and U.S. Pat. No. 5,179,017, by Axel et al.). Suitable selectable markers include genes that confer resistance to drugs such as G418, hygromycin or methotrexate, on a host cell into which the vector has been introduced. For example, the dihydrofolate reductase (DHFR) gene confers resistance to methotrexate and the neo gene confers resistance to G418. For efficient cloning the first 3 amino acids of the N-terminus of the heavy chains [EVQ] (SEQ ID NO:67 and SEQ ID NO:69) can also alternatively be expressed as [QVE], for example as a variant of the heavy chain of human IgG1 M048-D01-hIgG1 (SEQ ID NO:222). For efficient cloning the N-terminus of light chains can be extended by amino acid residues e.g. Alanin.

Transfection of the expression vector into a host cell can be carried out using standard techniques such as electroporation, calcium-phosphate precipitation, and DEAE-dextran transfection.

Suitable mammalian host cells for expressing the antibodies, antigen binding portions, or derivatives thereof provided herein include Chinese Hamster Ovary (CHO cells) [including dhfr—CHO cells, described in Urlaub and Chasin, (1980) Proc. Natl. Acad. Sci. USA 77:4216-4220, used with a DHFR selectable marker, e.g., as described in R. J. Kaufman and P. A, Sharp (1982) Mol. Biol. 159:601-621]], NSO myeloma cells, COS cells and SP2 cells. In some embodiments, the expression vector is designed such that the expressed protein is secreted into the culture medium in which the host cells are grown. The antibodies, antigen binding portions, or derivatives thereof can be recovered from the culture medium using standard protein purification methods.

Antibodies of the invention or an antigen-binding fragment thereof can be recovered and purified from recombinant cell cultures by well-known methods including, but not limited to ammonium sulfate or ethanol precipitation, acid extraction, Protein A chromatography, Protein G chromatography, anion or cation exchange chromatography, phospho-cellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Antibodies of the present invention or antigen-binding fragment thereof include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody of the present invention can be glycosylated or can be non-glycosylated. Such methods are described in many standard laboratory manuals, such as Sambrook, supra, Sections 17.37-17.42; Ausubel, supra, Chapters 10, 12, 13, 16, 18 and 20.

Therefore an embodiment of the present invention are also host cells comprising the vector or a nucleic acid molecule, whereby the host cell can be a higher eukaryotic host cell, such as a mammalian cell, a lower eukaryotic host cell, such as a yeast cell, and may be a prokaryotic cell, such as a bacterial cell.

Another embodiment of the present invention is a method of using the host cell to produce an antibody and antigen binding fragments, comprising culturing the host cell under suitable conditions and recovering said antibody.

Therefore another embodiment of the present invention is the production of the antibodies according to this invention (for example antibody M048-D01-hIgG1) with the host cells of the present invention and purification of these antibodies to at least 95% homogeneity by weight.

### Therapeutic Methods

Therapeutic methods involve administering to a subject in need of treatment a therapeutically effective amount of an antibody or antigen-binding fragment thereof contemplated by the invention. A “therapeutically effective” amount hereby is defined as the amount of an antibody or antigen-binding fragment that is of sufficient quantity to deplete FGFR2-positive cells in a treated area of a subject—either as a single dose or according to a multiple dose regimen, alone or in combination with other agents, which leads to the alleviation of an adverse condition, yet which amount is toxicologically tolerable. The subject may be a human or non-human animal (e.g., rabbit, rat, mouse, dog, monkey or other lower-order primate).

An antibody of the invention or antigen-binding fragment thereof might be co-administered with known medicaments, and in some instances the antibody might itself be modified. For example, an antibody could be conjugated to a cytotoxic agent or radioisotope to potentially further increase efficacy.

Antibodies of the present invention may be administered as the sole pharmaceutical agent or in combination with one or more additional therapeutic agents where the combination causes no unacceptable adverse effects. This combination therapy includes administration of a single pharmaceutical dosage formulation which contains an antibody of the invention and one or more additional therapeutic agents, as well as administration of an antibody of the invention and each additional therapeutic agent in its own separate pharmaceutical dosage formulation. For example, an antibody of the invention and a therapeutic agent may be administered to the patient together in a single oral dosage composition such as a tablet or capsule, or each agent may be administered in separate dosage formulations.

Where separate dosage formulations are used, an antibody of the invention and one or more additional therapeutic agents may be administered at essentially the same time (e.g., concurrently) or at separately staggered times (e.g., sequentially).

In particular, antibodies of the present invention may be used in fixed or separate combination with other anti-tumor agents such as alkylating agents, anti-metabolites, plant-derived anti-tumor agents, hormonal therapy agents, topoisomerase inhibitors, camptothecin derivatives, kinase inhibitors, targeted drugs, antibodies, interferons and/or biological response modifiers, anti-angiogenic compounds, and other anti-tumor drugs. In this regard, the following is a non-limiting list of examples of secondary agents that may be used in combination with the antibodies of the present invention:

Alkylating agents include, but are not limited to, nitrogen mustard N-oxide, cyclophosphamide, ifosfamide, thiotepa, ranimustine, nimustine, temozolomide, altretamine, apaziquone, brostallicin, bendamustine, carmustine, estramustine, fotemustine, glufosfamide, mafosfamide, bendamustin, and mitolactol; platinum-coordinated alkylating compounds include, but are not limited to, cisplatin, carboplatin, eptaplatin, lobaplatin, nedaplatin, oxaliplatin, and satraplatin;

Anti-metabolites include, but are not limited to, methotrexate, 6-mercaptopurine riboside, mercaptopurine, 5-fluorouracil alone or in combination with leucovorin, tegafur, doxifluridine, carmofur, cytarabine, cytarabine ocfosfate, enocitabine, gemcitabine, fludarabin, 5-azacitidine, capecitabine, cladribine, clofarabine, decitabine, eflornithine, ethynylcytidine, cytosine arabinoside, hydroxyurea, melphalan, nelarabine, nolatrexed, ocfosfite, disodium premetrexed, pentostatin, pelitrexol, raltitrexed, triapine, trimetrexate, vidarabine, vincristine, and vinorelbine;

Hormonal therapy agents include, but are not limited to, exemestane, Lupron, anastrozole, doxercalciferol, fadrozole, formestane, 11-beta hydroxysteroid dehydrogenase 1 inhibitors, 17-alpha hydroxylase/17,20 lyase inhibitors such as abiraterone acetate, 5-alpha reductase inhibitors such as finasteride and epristeride, anti-estrogens such as tamoxifen citrate and fulvestrant, Trelstar, toremifene, raloxifene, lasofoxifene, letrozole, anti-androgens such as bicalutamide, flutamide, mifepristone, nilutamide, Casodex, and anti-progesterones and combinations thereof.

Plant-derived anti-tumor substances include, e.g., those selected from mitotic inhibitors, for example epothilones such as sagopilone, ixabepilone and epothilone B, vinblastine, vinflunine, docetaxel, and paclitaxel;

Cytotoxic topoisomerase inhibiting agents include, but are not limited to, aclarubicin, doxorubicin, amonafide, belotecan, camptothecin, 10-hydroxycamptothecin, 9-aminocamptothecin, diflomotecan, irinotecan, topotecan, edotecarin, epimbicin, etoposide, exatecan, gimatecan, lurtotecan, mitoxantrone, pirambicin, pixantrone, rubitecan, sobuzoxane, tafluposide, and combinations thereof;

Immunologicals include interferons such as interferon alpha, interferon alpha-2a, interferon alpha-2b, interferon beta, interferon gamma-1a and interferon gamma-n1, and other immune enhancing agents such as L19-IL2 and other IL2 derivatives, filgrastim, lentinan, sizofilan, TheraCys, ubenimex, aldesleukin, alemtuzumab, BAM-002, dacarbazine, daclizumab, denileukin, gemtuzumab, ozogamicin, ibritumomab, imiquimod, lenograstim, lentinan, melanoma vaccine (Corixa), molgramostim, sargramostim, tasonermin, tecleukin, thymalasin, tositumomab, Vimlizin, epratuzumab, mitumomab, oregovomab, pemtumomab, and Provenge;

Biological response modifiers are agents that modify defense mechanisms of living organisms or biological responses such as survival, growth or differentiation of tissue cells to direct them to have anti-tumor activity; such agents include, e.g., krestin, lentinan, sizofiran, picibanil, ProMune, and ubenimex;

Anti-angiogenic compounds include, but are not limited to, acitretin, aflibercept, angiostatin, aplidine, asentar, axitinib, bevacizumab, brivanib alaninat, cilengtide, combretastatin, endostatin, fenretinide, halofuginone, pazopanib, ranibizumab, rebimastat, recentin, regorafenib, removab, revlimid, sorafenib, squalamine, sunitinib, telatinib, thalidomide, ukrain, vatalanib, and vitaxin;

Antibodies include, but are not limited to, trastuzumab, cetuximab, bevacizumab, rituximab, ticilimumab, ipilimumab, lumiliximab, catumaxomab, atacicept, oregovomab, and alemtuzumab;

VEGF inhibitors such as, e.g., sorafenib, regorafenib, bevacizumab, sunitinib, recentin, axitinib, aflibercept, telatinib, brivanib alaninate, vatalanib, pazopanib, and ranibizumab;

EGFR (HER1) inhibitors such as, e.g., cetuximab, panitumumab, vectibix, gefitinib, erlotinib, and Zactima;

HER2 inhibitors such as, e.g., lapatinib, tratuzumab, and pertuzumab;

mTOR inhibitors such as, e.g., temsirolimus, sirolimus/Rapamycin, and everolimus;

c-Met inhibitors;

PI3K and AKT inhibitors;

CDK inhibitors such as roscovitine and flavopiridol;

Spindle assembly checkpoints inhibitors and targeted anti-mitotic agents such as PLK inhibitors, Aurora inhibitors (e.g. Hesperadin), checkpoint kinase inhibitors, and KSP inhibitors;

HDAC inhibitors such as, e.g., panobinostat, vorinostat, MS275, belinostat, and LBH589;

HSP90 and HSP70 inhibitors;

Proteasome inhibitors such as bortezomib and carfilzomib;

Serine/threonine kinase inhibitors including MEK inhibitors and Raf inhibitors such as sorafenib;

Farnesyl transferase inhibitors such as, e.g., tipifarnib;

Tyrosine kinase inhibitors including, e.g., dasatinib, nilotibib, regorafenib, bosutinib, sorafenib, bevacizumab, sunitinib, cediranib, axitinib, aflibercept, telatinib, imatinib mesylate, brivanib alaninate, pazopanib, ranibizumab, vatalanib, cetuximab, panitumumab, vectibix, gefitinib, erlotinib, lapatinib, tratuzumab, pertuzumab, and c-Kit inhibitors;

Vitamin D receptor agonists;

Bcl-2 protein inhibitors such as obatoclax, oblimersen sodium, and gossypol;

Cluster of differentiation 20 receptor antagonists such as, e.g., rituximab;

Ribonucleotide reductase inhibitors such as, e.g., gemcitabine;

Tumor necrosis apoptosis inducing ligand receptor 1 agonists such as, e.g., mapatumumab;

5-Hydroxytryptamine receptor antagonists such as, e.g., rEV598, xaliprode, palonosetron hydrochloride, granisetron, Zindol, and AB-1001;

Integrin inhibitors including alpha5-beta1 integrin inhibitors such as, e.g., E7820, JSM 6425, volociximab, and endostatin;

Androgen receptor antagonists including, e.g., nandrolone decanoate, fluoxymesterone, Android, Prost-aid, andromustine, bicalutamide, flutamide, apo-cyproterone, apo-flutamide, chlormadinone acetate, Androcur, Tabi, cyproterone acetate, and nilutamide;

Aromatase inhibitors such as, e.g., anastrozole, letrozole, testolactone, exemestane, aminoglutethimide, and formestane;

Matrix metalloproteinase inhibitors;

Other anti-cancer agents including, e.g., alitretinoin, ampligen, atrasentan bexarotene, bortezomib, bosentan, calcitriol, exisulind, fotemustine, ibandronic acid, miltefosine, mitoxantrone, 1-asparaginase, procarbazine, dacarbazine, hydroxycarbamide, pegaspargase, pentostatin, tazaroten, velcade, gallium nitrate, canfosfamide, darinaparsin, and tretinoin.

In a preferred embodiment, the antibodies of the present invention may be used in combination with chemotherapy (i.e. cytotoxic agents), anti-hormones and/or targeted therapies such as other kinase inhibitors (for example, EGFR inhibitors), mTOR inhibitors and angiogenesis inhibitors.

The compounds of the present invention may also be employed in cancer treatment in conjunction with radiation therapy and/or surgical intervention.

An antibody of the invention or antigen-binding fragment thereof might in some instances itself be modified. For example, an antibody could be conjugated to any of but not limited to the compounds mentioned above or any radioisotope to potentially further increase efficacy. Furthermore, the antibodies of the invention may be utilized, as such or in compositions, in research and diagnostics, or as analytical reference standards, and the like, which are well known in the art.

The inventive antibodies or antigen-binding fragments thereof can be used as a therapeutic or a diagnostic tool in a variety of situations with aberrant FGFR2-signaling, e.g. cell proliferative disorders such as cancer or fibrotic diseases. Disorders and conditions particularly suitable for treatment with an antibody of the inventions are solid tumors, such as cancers of the breast, respiratory tract, brain, reproductive organs, digestive tract, urinary tract, eye, liver, skin, head and neck, thyroid, parathyroid, and their distant metastases. Those disorders also include lymphomas, sarcomas and leukemias.

Tumors of the digestive tract include, but are not limited to anal, colon, colorectal, esophageal, gallbladder, gastric, pancreatic, rectal, small-intestine, and salivary gland cancers.

Examples of esophageal cancer include, but are not limited to esophageal cell carcinomas and adenocarcinomas, as well as squamous cell carcinomas, leiomyosarcoma, malignant melanoma, rhabdomyosarcoma and lymphoma.

Examples of gastric cancer include, but are not limited to intestinal type and diffuse type gastric adenocarcinoma.

Examples of pancreatic cancer include, but are not limited to ductal adenocarcinoma, adenosquamous carcinomas and pancreatic endocrine tumors.

Examples of breast cancer include, but are not limited to triple negative breast cancer, invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, glioblastoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal and vulvar cancer, as well as sarcoma of the uterus.

Examples of ovarian cancer include, but are not limited to serous tumour, endometrioid tumor, mucinous cystadenocarcinoma, granulosa cell tumor, Sertoli-Leydig cell tumor and arrhenoblastoma

Examples of cervical cancer include, but are not limited to squamous cell carcinoma, adenocarcinoma, adenosquamous carcinoma, small cell carcinoma, neuroendocrine tumour, glassy cell carcinoma and villoglandular adenocarcinoma.

Tumors of the urinary tract include, but are not limited to bladder, penile, kidney, renal pelvis, ureter, urethral, and hereditary and sporadic papillary renal cancers.

Examples of kidney cancer include, but are not limited to renal cell carcinoma, urothelial cell carcinoma, juxtaglomerular cell tumor (reninoma), angiomyolipoma, renal oncocytoma, Bellini duct carcinoma, clear-cell sarcoma of the kidney, mesoblastic nephroma and Wilms' tumor.

Examples of bladder cancer include, but are not limited to transitional cell carcinoma, squamous cell carcinoma, adenocarcinoma, sarcoma and small cell carcinoma.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to squamous cell cancer of the head and neck, laryngeal, hypopharyngeal, nasopharyngeal, oropharyngeal cancer, lip and oral cavity cancer, and squamous cell cancer.

Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia. In a preferred embodiment, the antibodies or antigen-binding fragments thereof of the invention are suitable for a therapeutic or diagnostic method for the treatment or diagnosis of a cancer disease comprised in a group consisting of gastric cancer, breast cancer, pancreatic cancer, colorectal cancer, kidney cancer, prostate cancer, ovarian cancer, cervical cancers, lung cancer, endometrial cancer, esophageal cancer, head and neck cancer, hepatocellular carcinoma, melanoma and bladder cancer. In addition, the inventive antibodies or antigen-binding fragments thereof can also be used as a therapeutic or a diagnostic tool in a variety of other disorders wherein FGFR2 is involved such as, but not limited to fibrotic diseases such as intraalveolar fibrosis, silica-induced pulmonary fibrosis, experimental lung fibrosis, idiopathic lung fibrosis, renal fibrosis, as well as lymphangioleiomyomatosis, polycystic ovary syndrome, acne, psoriasis, cholesteatoma, cholesteatomatous chronic otitis media, periodontitis, solar lentigines, bowel disease, atherosclerosis or endometriosis.

The disorders mentioned above have been well characterized in humans, but also exist with a similar etiology in other animals, including mammals, and can be treated by administering pharmaceutical compositions of the present invention.

To treat any of the foregoing disorders, pharmaceutical compositions for use in accordance with the present invention may be formulated in a conventional manner using one or more physiologically acceptable carriers or excipients. An antibody of the invention or antigen-binding fragment thereof can be administered by any suitable means, which can vary, depending on the type of disorder being treated. Possible administration routes include parenteral (e.g., intramuscular, intravenous, intra-arterial, intraperitoneal, or subcutaneous), intrapulmonary and intranasal, and, if desired for local immunosuppressive treatment, intralesional administration. In addition, an antibody of the invention might be administered by pulse infusion, with, e.g., declining doses of the antibody. Preferably, the dosing is given by injections, most preferably intravenous or subcutaneous injections, depending in part on whether the administration is brief or chronic. The amount to be administered will depend on a variety of factors such as the clinical symptoms, weight of the individual, whether other drugs are administered. The skilled artisan will recognize that the route of administration will vary depending on the disorder or condition to be treated.

Determining a therapeutically effective amount of the novel polypeptide, according to this invention, largely will depend on particular patient characteristics, route of administration, and the nature of the disorder being treated. General guidance can be found, for example, in the publications of the International Conference on Harmonization and in REMINGTON'S PHARMACEUTICAL SCIENCES, chapters 27 and 28, pp. 484-528 (18th ed., Alfonso R. Gennaro, Ed., Easton, Pa.: Mack Pub. Co., 1990). More specifically, determining a therapeutically effective amount will depend on such factors as toxicity and efficacy of the medicament. Toxicity may be determined using methods well known in the art and found in the foregoing references. Efficacy may be determined utilizing the same guidance in conjunction with the methods described below in the Examples.

### Diagnostic Methods

FGFR2 antibodies or antigen-binding fragments thereof can be used for detecting the presence of FGFR2-expressing tumors. The presence of FGFR2-containing cells or shed FGFR2 within various biological samples, including serum, and tissue biopsy specimens, may be detected with FGFR2 antibodies. In addition, FGFR2 antibodies may be used in various imaging methodologies such as immunoscintigraphy with a ⁹⁹Tc (or other isotope) conjugated antibody. For example, an imaging protocol similar to the one recently described using a ¹¹¹In conjugated anti-PSMA antibody may be used to detect pancreatic or ovarian carcinomas (Sodee et al., Clin. Nuc. Mod. 2|: 759-766, 1997). Another method of detection that can be used is positron emitting tomography by conjugating the antibodies of the invention with a suitable isotope (see Herzog et al., J. Nucl. Med. 34:2222-2226, 1993).

### Pharmaceutical Compositions and Administration

An embodiment of the present invention are pharmaceutical compositions which comprise FGFR2 antibodies or antigen-binding fragment thereof, alone or in combination with at least one other agent, such as stabilizing compound, which may be administered in any sterile, biocompatible pharmaceutical carrier, including, but not limited to, saline, buffered saline, dextrose, and water. A further embodiment are pharmaceutical compositions comprising a FGFR2 binding antibody or antigen-binding fragment thereof and a further pharmaceutically active compound that is suitable to treat FGFR2 related diseases such as cancer. Any of these molecules can be administered to a patient alone, or in combination with other agents, drugs or hormones, in pharmaceutical compositions where it is mixed with excipient(s) or pharmaceutically acceptable carriers. In one embodiment of the present invention, the pharmaceutically acceptable carrier is pharmaceutically inert.

The present invention also relates to the administration of pharmaceutical compositions. Such administration is accomplished orally or parenterally. Methods of parenteral delivery include topical, intra-arterial (directly to the tumor), intramuscular, subcutaneous, intramedullary, intrathecal, intraventricular, intravenous, intraperitoneal, or intranasal administration. In addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Ed. Maack Publishing Co, Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl, cellulose, hydroxypropylmethylcellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinyl pyrrolidone, carbopol gel, polyethylene glycol and/or titanium dioxide, lacquer solutions, and suitable organic solvents or solvent mixtures, Dyestuffs or pigments may be added to the tablets or dragee coatings for product identification or to characterize the quantity of active compound, i.e. dosage.

Pharmaceutical preparations that can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer's solution, or physiologically buffered saline. Aqueous injection suspensions may contain substances that increase viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

### Kits

The invention further relates to pharmaceutical packs and kits comprising one or more containers filled with one or more of the ingredients of the aforementioned compositions of the invention. Associated with such container(s) can be a notice in the form prescribed by a governmental agency regulating the manufacture, use or sale of pharmaceuticals or biological products, reflecting approval by the agency of the manufacture, use or sale of the product for human administration.

In another embodiment, the kits may contain DNA sequences encoding the antibodies of the invention. Preferably the DNA sequences encoding these antibodies are provided in a plasmid suitable for transfection into and expression by a host cell. The plasmid may contain a promoter (often an inducible promoter) to regulate expression of the DNA in the host cell. The plasmid may also contain appropriate restriction sites to facilitate the insertion of other DNA sequences into the plasmid to produce various antibodies. The plasmids may also contain numerous other elements to facilitate cloning and expression of the encoded proteins. Such elements are well known to those of skill in the art and include, for example, selectable markers, initiation codons, termination codons, and the like.

### Manufacture and Storage.

The pharmaceutical compositions of the present invention may be manufactured in a manner that is known in the art, e.g., by means of conventional mixing, dissolving, granulating, dragee-making, levigating, emulsifying, encapsulating, entrapping or lyophilizing processes.

The pharmaceutical composition may be provided as a salt and can be formed with acids, including by not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

After pharmaceutical compositions comprising a compound of the invention formulated in an acceptable carrier have been prepared, they can be placed in an appropriate container and labeled for treatment of an indicated condition. For administration of FGFR2 antibodies or antigen-binding fragment thereof, such labeling would include amount, frequency and method of administration.

### Therapeutically Effective Dose.

Pharmaceutical compositions suitable for use in the present invention include compositions wherein the active ingredients are contained in an effective amount to achieve the intended purpose, i.e. treatment of a particular disease state characterized by FGFR2 expression. The determination of an effective dose is well within the capability of those skilled in the art.

For any compound, the therapeutically effective dose can be estimated initially either in cell culture assays, e.g., neoplastic cells, or in animal models, usually mice, rabbits, dogs, pigs or monkeys. The animal model is also used to achieve a desirable concentration range and route of administration. Such information can then be used to determine useful doses and routes for administration in humans.

A therapeutically effective dose refers to that amount of antibody or antigen-binding fragment thereof, that ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, ED50/LD50. Pharmaceutical compositions that exhibit large therapeutic indices are preferred. The data obtained from cell culture assays and animal studies are used in formulating a range of dosage for human use. The dosage of such compounds lies preferably within a range of circulating concentrations what include the ED50 with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors that may be taken into account include the severity of the disease state, e.g., tumor size and location; age, weight and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Normal dosage amounts may vary from 0.1 to 100,000 micrograms, up to a total dose of about 2 g, depending upon the route of administration. Guidance as to particular dosages and methods of delivery is provided in the literature. See U.S. Pat. No. 4,657,760; 5,206,344; or 5,225,212. Those skilled in the art will employ different formulations for polynucleotides than for proteins or their inhibitors. Similarly, delivery of polynucleotides or polypeptides will be specific to particular cells, conditions, locations, etc. Preferred specific activities for a radiolabelled antibody may range from 0.1 to 10 mCi/mg of protein (Riva et al., Clin. Cancer Res. 5:3275-3280, 1999; Ulaner et al., 2008 Radiology 246(3):895-902)

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

All examples were carried out using standard techniques, which are well known and routine to those of skill in the art, except where otherwise described in detail. Routine molecular biology techniques of the following examples can be carried out as described in standard laboratory manuals, such as Sambrook et al., Molecular Cloning: A Laboratory Manual, 2nd Ed.; Cold Spring Harbor Laboratory Press, Cold Spring Harbor, N.Y., 1989.

A preferred embodiment of the invention is:
A. An isolated antibody or antigen-binding fragment thereof which reduces the cell surface expression of FGFR2 after binding to FGFR2 in cell lines SNU16 (ATCC-CRL-5974) and MFM223 (ECACC-98050130) which overexpress FGFR2 and in cell lines AN3-CA (DSMZ-ACC 267) and MFE-296 (ECACC-98031101) which express mutated FGFR2.
B. An isolated antibody or antigen-binding fragment thereof according to claim A wherein the antibody or antigen-binding fragment thereof specifically binds to the extracellular N-terminal epitope (¹RPSFSLVEDTTLEPE¹⁵) of FGFR2 as presented by (SEQ ID NO:63).
C. An isolated antibody or antigen-binding fragment thereof according to claim B wherein binding of the antibody to the extracellular N-terminal epitope (SEQ ID NO:63) is mediated by at least one epitope residue selected from the group of residues consisting of Arg 1, Pro 2, Phe 4, Ser 5, Leu 6, and Glu 8.
D. An isolated antibody or antigen-binding fragment thereof according to any one of claims B-C wherein the antibody or antigen-binding fragment thereof loses more than 50% of its ELISA signal by changing of at least one of the amino acid residues in the N-terminal epitope (¹RPSFSLVEDTTLEPE¹⁵) of FGFR2 into an Alanine
   a) said residue selected from the group Pro 2, Leu 6 and Glu 8, or
   b) said residue selected from the group Arg 1, Pro 2, Phe 4 and Ser 5.
E. The antibody or antigen-binding fragment according to any one of claims A to D, wherein the antibody or antigen-binding fragment competes in binding to FGFR2 with at least one antibody selected from the group “M048-D01”, “M047-D08”, “M017-B02”, “M021-H02”, “M054-A05” “M054-D03”, “TPP-1397”, “TPP-1398”, “TPP-1399”, “TPP-1400”, “TPP-1401”, “TPP-1402”, “TPP-1403”, “TPP-1406”, “TPP-1407”, “TPP-1408”, “TPP-1409”, “TPP-1410”, “TPP-1411”, “TPP-1412”, and “TPP-1415”.
F. The antibody or antigen-binding fragment according to claim E, wherein the amino acid sequence of the antibody or antigen-binding fragment is at least 50%, 55%, 60% 70%, 80%, 90, or 95% identical to at least one CDR sequence of “M048-D01”, “M047-D08”, “M017-B02”, “M021-H02”, “M054-A05”, “M054-D03”, “TPP-1397”, “TPP-1398”, “TPP-1399”, “TPP-1400”, “TPP-1401”, “TPP-1402”, “TPP-1403”, “TPP-1406”, “TPP-1407”, “TPP-1408”, “TPP-1409”, “TPP-1410”, “TPP-1411”, “TPP-1412”, or “TPP-1415”, or at least 50%, 60%, 70%, 80%, 90%, 92% or 95% identical to the VH or VL sequence of “M048-D01”, “M047-D08”, “M017-B02”, “M021-002”, “M054-A05”, “M054-D03”, “TPP-1397”, “TPP-1398”, “TPP-1399”, “TPP-1400”, “TPP-1401”, “TPP-1402”, “TPP-1403”, “TPP-1406”, “TPP-1407”, “TPP-1408”, “TPP-1409”, “TPP-1410”, “TPP-1411”, “TPP-1412”, or “TPP-1415”.
G. The antibody or antigen-binding fragment according to any one of claims E to F, wherein the antibody or antigen-binding fragment comprises at least one CDR sequence or at least one variable heavy chain or light chain sequence as depicted in Table 9 and Table 10.
H. The antibody or antigen-binding fragment according to claim A to G comprising
   a) the variable heavy chain CDR sequences as presented by SEQ ID NO: 5-7 and the variable light chain CDR sequences presented by SEQ ID NO: 8-10, or
   b) the variable heavy chain CDR sequences as presented by SEQ ID NO: 15-17 and the variable light chain CDR sequences presented by SEQ ID NO: 18-20, or
   c) the variable heavy chain CDR sequences as presented by SEQ ID NO: 25-27 and the variable light chain CDR sequences presented by SEQ ID NO: 28-30, or
   d) the variable heavy chain CDR sequences as presented by SEQ ID NO: 35-37 and the variable light chain CDR sequences presented by SEQ ID NO: 38-40, or
   e) the variable heavy chain CDR sequences as presented by SEQ ID NO: 45-47 and the variable light chain CDR sequences presented by SEQ ID NO: 48-50, or
   f) the variable heavy chain CDR sequences as presented by SEQ ID NO: 55-57 and the variable light chain CDR sequences presented by SEQ ID NO: 58-60, or
   g) the variable heavy chain CDR sequences as presented by SEQ ID NO: 75-77 and the variable light chain CDR sequences presented by SEQ ID NO: 78-80, or
   h) the variable heavy chain CDR sequences as presented by SEQ ID NO: 85-87 and the variable light chain CDR sequences presented by SEQ ID NO: 88-90, or
   i) the variable heavy chain CDR sequences as presented by SEQ ID NO: 95-97 and the variable light chain CDR sequences presented by SEQ ID NO: 98-100, or
   j) the variable heavy chain CDR sequences as presented by SEQ ID NO: 105-107 and the variable light chain CDR sequences presented by SEQ ID NO: 108-110, or
   k) the variable heavy chain CDR sequences as presented by SEQ ID NO: 115-117 and the variable light chain CDR sequences presented by SEQ ID NO: 118-120, or
   l) the variable heavy chain CDR sequences as presented by SEQ ID NO: 125-127 and the variable light chain CDR sequences presented by SEQ ID NO: 128-130, or
   m) the variable heavy chain CDR sequences as presented by SEQ ID NO: 135-137 and the variable light chain CDR sequences presented by SEQ ID NO: 138-140, or
   n) the variable heavy chain CDR sequences as presented by SEQ ID NO: 145-147 and the variable light chain CDR sequences presented by SEQ ID NO: 148-150, or
   o) the variable heavy chain CDR sequences as presented by SEQ ID NO: 155-157 and the variable light chain CDR sequences presented by SEQ ID NO: 158-160, or
   p) the variable heavy chain CDR sequences as presented by SEQ ID NO: 165-167 and the variable light chain CDR sequences presented by SEQ ID NO: 168-170, or
   q) the variable heavy chain CDR sequences as presented by SEQ ID NO: 175-177 and the variable light chain CDR sequences presented by SEQ ID NO: 178-180, or
   r) the variable heavy chain CDR sequences as presented by SEQ ID NO: 185-187 and the variable light chain CDR sequences presented by SEQ ID NO: 188-190, or
   s) the variable heavy chain CDR sequences as presented by SEQ ID NO: 195-197 and the variable light chain CDR sequences presented by SEQ ID NO: 198-200, or
   t) the variable heavy chain CDR sequences as presented by SEQ ID NO: 205-207 and the variable light chain CDR sequences presented by SEQ ID NO: 208-210, or
   u) the variable heavy chain CDR sequences as presented by SEQ ID NO: 215-217 and the variable light chain CDR sequences presented by SEQ ID NO: 218-220.
I. The antibody or antigen-binding fragment according to claims A-H comprising
   a) a variable heavy chain sequence as presented by SEQ ID NO:1 and a variable light chain sequences as presented by SEQ ID NO:2, or
   b) a variable heavy chain sequence as presented by SEQ ID NO:11 and a variable light chain sequences as presented by SEQ ID NO:12, or
   c) a variable heavy chain sequence as presented by SEQ ID NO:21 and a variable light chain sequences as presented by SEQ ID NO:22, or
   d) a variable heavy chain sequence as presented by SEQ ID NO:31 and a variable light chain sequences as presented by SEQ ID NO:32, or
   e) a variable heavy chain sequence as presented by SEQ ID NO:41 and a variable light chain sequences as presented by SEQ ID NO:42, or
   f) a variable heavy chain sequence as presented by SEQ ID NO:51 and a variable light chain sequences as presented by SEQ ID NO:52, or
   g) a variable heavy chain sequence as presented by SEQ ID NO:73 and a variable light chain sequences as presented by SEQ ID NO:74, or
   h) a variable heavy chain sequence as presented by SEQ ID NO:83 and a variable light chain sequences as presented by SEQ ID NO:84, or
   i) a variable heavy chain sequence as presented by SEQ ID NO:93 and a variable light chain sequences as presented by SEQ ID NO:94, or
   j) a variable heavy chain sequence as presented by SEQ ID NO:103 and a variable light chain sequences as presented by SEQ ID NO:104, or
   k) a variable heavy chain sequence as presented by SEQ ID NO:113 and a variable light chain sequences as presented by SEQ ID NO:114, or
   l) a variable heavy chain sequence as presented by SEQ ID NO:123 and a variable light chain sequences as presented by SEQ ID NO:124, or
   m) a variable heavy chain sequence as presented by SEQ ID NO:133 and a variable light chain sequences as presented by SEQ ID NO:134, or
   n) a variable heavy chain sequence as presented by SEQ ID NO:143 and a variable light chain sequences as presented by SEQ ID NO:144, or
   o) a variable heavy chain sequence as presented by SEQ ID NO:153 and a variable light chain sequences as presented by SEQ ID NO:154, or
   p) a variable heavy chain sequence as presented by SEQ ID NO:163 and a variable light chain sequences as presented by SEQ ID NO:164, or
   q) a variable heavy chain sequence as presented by SEQ ID NO:173 and a variable light chain sequences as presented by SEQ ID NO:174, or
   r) a variable heavy chain sequence as presented by SEQ ID NO:183 and a variable light chain sequences as presented by SEQ ID NO:184, or
   s) a variable heavy chain sequence as presented by SEQ ID NO:193 and a variable light chain sequences as presented by SEQ ID NO:194, or
   t) a variable heavy chain sequence as presented by SEQ ID NO:203 and a variable light chain sequences as presented by SEQ ID NO:204, or
   u) a variable heavy chain sequence as presented by SEQ ID NO:213 and a variable light chain sequences as presented by SEQ ID NO:214.
J. The antibody according to any one of the preceding claims, which is an IgG antibody.
K. The antigen-binding fragment according to any one of the preceding claims, which is an scFv, Fab, Fab′ fragment or a F(ab′)₂ fragment.
L. The antibody or antigen-binding fragment according to any one of the preceding claims, which is a monoclonal antibody or antigen-binding fragment.
M. The antibody or antigen-binding fragment according to any one of the preceding claims, which is human, humanized or chimeric antibody or antigen-binding fragment.
N. An antibody-drug conjugate, comprising an antibody or antigen binding fragment thereof according to claims A to M.
O. An isolated nucleic acid sequence that encodes the antibody or antigen-binding fragment according to claims A to M.
P. A vector comprising a nucleic acid sequence according to claim O.
Q. An isolated cell expressing an antibody or antigen-binding fragment according to any one of the claims A-M and for comprising a nucleic acid according to claim O or a vector according to claim P.
R. An isolated cell according to claim Q, wherein said cell is a prokaryotic or an eukaryotic cell.
S. A method of producing an antibody or antigen-binding fragment according to any one of the claims A-M comprising culturing of a cell according to claim R and purification of the antibody or antigen-binding fragment.
T. An antibody or antigen-binding fragment according to claims A-M or an antibody-drug conjugate according to claim N as a medicament.
U. An antibody or antigen antigen-binding fragment according to claims A-M as a diagnostic agent.
V. An antibody or antigen-binding fragment according to claims A-M or an antibody-drug conjugate according to claim N as a medicament for the treatment of cancer.
W. A pharmaceutical composition comprising an antibody or antigen-binding fragment according to claims A-M or an antibody-drug conjugate according to claim N.
X. A combination of a pharmaceutical composition according to claim W and one or more therapeutically active compounds.
Y. A method for treating a disorder or condition associated with the undesired presence of FGFR2, comprising administering to a subject in need thereof an effective amount of the pharmaceutical composition according to claim W or a combination according to claim X.

### EXAMPLES

### Example 1

### Antibody Generation from n-CoDeR Libraries

### Tools Used for Phage Selections:

Recombinant proteins used for the isolation of human antibodies of the present invention were obtained from R&D Systems and are listed in Table 1. All variants used were present as Fc-fusion proteins in carrier free preparations. hTRAIL-Fc served as depletion agent to avoid Fc binder. Proteins were biotinylated according to manufacturer's instructions using an approximately 2-fold molar excess of biotin-LC-NHS (Pierce; Cat. No. 21347) and desalted using Zeba desalting columns (Pierce; Cat. No. 89889).

TABLE 1
List of recombinant proteins used in phage
selections and screening

| Protein | Origin | Cat. No. (R&D Systems) |
|---|---|---|
| hFGFR2β-Fc (IIIb) | Human | 665-FR |
| mFGFR2β-Fc (IIIb) | Murine | 708-MF |
| hFGFR2α-Fc (IIIb) | Human | 663-FR |
| hFGFR2β-Fc (IIIc) | Human | 684-FR |
| hTRAIL-Fc | Human | 630-TR |

For phage selections on cells the human gastric carcinoma cell line KATO III (ATCC HTB-103) was employed, displaying native FGFR2 on its cell surface.

### Phage Selections:

The isolation of human antibodies of the present invention or antigen binding fragments thereof was performed by phage display technology employing the naive Fab antibody library n-CoDeR of BioInvent International AB (Lund, Sweden; described in Söderling et al., Nat. Biotech. 2000, 18:853-856), which is a Fab library in which all six CDRs are diversified. As summarized in Table 2, three different strategies for the selection of inventive antibodies were employed.

TABLE 2
Summary of selection strategies

| Round of | | | |
|---|---|---|---|
| selection: | Strategy I | Strategy II | Strategy III |
| 1 | 200 nM biotinylated hFGFR2β-Fc (IIIb) | | |
| 2 | KATO III cells | 200 nM biotinylated | 200 nM |
| | | mFGFR2β-Fc (IIIb) | biotinylated |
| | | | hFGFR2β-Fc (IIIc) |
| 3 | 100 nM biotinylated | 100 nM biotinylated | 200 nM |
| | hFGFR2β-Fc (IIIb) | hFGFR2α-Fc (IIIb) | biotinylated |
| | | | hFGFR2α-Fc (IIIb) |
| 4 | KATO III cells | 100 nM biotinylated | 200 nM |
| | | mFGFR2β-Fc (IIIb) | biotinylated |
| | | | hFGFR2β-Fc (IIIc) |

Standard buffers used in this example are:
1×PBS: from Sigma (D5652-501)
PBST: 1×PBS supplemented with 0.05% Tween20 (Sigma, P7949)
Blocking buffer: PBST supplemented with 3% BSA (Sigma A4503)
Precipitation buffer: 20% PEG (Calbiochem, 528877) in 2.5 M NaCl
FACS-buffer: PBS supplemented with 3% FBS (GIBCO, 10082) and 0.01% NaN₃ (Sigma, 71289)

Briefly, an aliquot of the Fab antibody library was centrifuged at r.t for 5 min, the resulting pellet was resuspended in 40 ml PBS and precipitated by addition of precipitation buffer followed by an incubation on ice for 1 h and a centrifugation step (1 h at 4000 rpm). The precipitated library was subsequently resuspended in 1 ml blocking buffer and incubated at r.t. for 30 min.

Meanwhile, aliquots of streptavidin-coated Dynabeads M280 (Invitrogen, 11206D) were prepared by washing 3 times with PBS for 30 min on an end-to-end rotator. After that some aliquots were mixed with 200 nM biotinylated TRAIL-Fc protein while the remaining were mixed with the biotinylated target protein as indicated in Table 2. The mixtures were incubated at r.t. on an end-to-end rotator for 30 min and subsequently washed 3 times in 1 ml PBS. Coated beads were finally blocked by resuspension in 1 ml blocking buffer followed by collection of the beads and removal of the supernatant.

For depletion of unwanted Fc binders the blocked library (described above) was added to blocked Dynabeads coated with TRAIL-Fc and incubated at r.t. for 30 min while rotating. After collection of the beads on a magnetic rack, the supernatant was mixed with blocked Dynabeads coated with target protein. After 60 min incubation on an end-to-end rotator the samples were washed 3 times with blocking buffer followed by 5 times washing with PBST. Bound phages were eluted by adding 100 μl triethanolamine solution (TEA, 100 mM). After 10 min incubation at r.t., samples were neutralized by adding 400 μl 1M Tris-Cl, pH 7.5.

Panning strategy I included 2 rounds of panning on whole cells as a source of target protein (see Table 2). For this purpose, KATO III cells were resuspended in ice cold FACS buffer at a density of 10⁷ cells per ml. An aliquot of rescued phages were added to 1 ml cell suspension and incubated at 4° C. by end-over-end rotation. Subsequently, cells were washed 10 times with 2.5 ml FACS buffer followed by an elution of bound phages with 300 μl 76 nM citric acid (pH 2.5). After 5 min incubation, cells were centrifuged for 5 min at 400 g and 4° C. and the supernatant was neutralized by adding 300 ml 1 M Tris-Cl, pH 7.5

Eluted phages were propagated and phage titers determined essentially as previously described (Cicortas Gunnarsson et al., Protein Eng Des Sel 2004; 17 (3): 213-21). Briefly, aliquots of the eluate solution were saved for titration experiments while the rest was used to transform exponentially growing E. coli TG1 (from Stratagene) for preparation of new phage stocks used in a second, third and fourth selection round according to the strategies depicted in Table 2. For each selection round, both input and output phages were titrated on exponentially growing E. coli TG1 and clones were picked from round 2 to 4 for analysis in Phage ELISA.

### Enzyme-Linked Immunosorbent Assay (ELISA):

### Phage ELISA:

Selected phages from different selection rounds were analyzed for specificity using phage ELISA. Briefly, phage expression was performed by adding 10 μl of over night culture (in LB-medium supplemented with 100 μg/ml ampicillin (Sigma, A5354), 1% glucose) to 100 μl fresh medium (LB-medium supplemented with 100 μg/ml ampicillin and 0.1% glucose (Sigma, G8769) and shaking at 250 rpm and 37° C. in 96-well MTP until an OD600 of 0.5 was reached. Subsequently 40 μl helper phage M13KO7 (Invitrogen, 420311) was added and samples were incubated for another 15 min at 37° C. without shaking. After addition of IPTG (f.c. of 0.5 mM; final volume 200 μl) cells were incubated over night at 30° C. while shaking at 200 rpm.

96-well ELISA-plates pre-coated with streptavidin (Pierce, 15500) were coated over night at 4° C. with 1 μg/ml biotinylated FGFR2-2β Fc (IIIb) or biotinylated TRAIL-Fc. The next day plates were washed 3 times with PBST, treated with blocking reagent, and washed again 3 times with PBST. Meanwhile, phage cultures were briefly centrifuged, than 125 μl of the supernatant was removed and mixed with 125 μl blocking buffer. After that 100 μl of the blocked phages were transferred per well and incubated for 1 h at r.t. After washing 3 times with PBST, anti M13 antibody coupled to HRP (GE Healthcare, 27-9421-01; 1:2500 diluted in PBST) was added and incubated for 1 h at r.t. Color reaction was developed by addition of 50 μl TMB (Invitrogen, 2023) and stopped after 5-15 min by adding 50 μl H₂SO₄ (Merck, 1120801000). Colorimetric reaction was recorded at 450 nM in a plate reader (Tecan).

Screening of sFabs by ELISA:

For the generation of soluble Fab fragments (sFabs) phagemid DNA from the selection rounds 3 and 4 was isolated and digested with restriction enzymes EagI (Fermentas, FD0334) and EcoRI (NEB, R0101L) according to the providers instructions in order to remove the gene III sequence. The resulting fragment was re-ligated and constructs were transformed into chemically competent E. coli Top10 using standard methods. Single clones were picked, transferred to 96-well plates containing LB-media (100 μg/ml ampicillin (Sigma, A5354), 1% glucose) and shaken ON at 250 rpm and 37° C. The next morning 10 ml of pre-culture was transferred to 150 μl fresh LB-media (100 μg/ml ampicillin (Sigma, A5354), 0.1% glucose) until an OD600 of 0.5 was reached. After that sFab production was induced by the addition of IPTG (f.c. 0.5 mM) and incubation was continued over night at 30° C. while shaking at 200 rpm. Next morning 50 μl BEL-buffer (24.7 g/l boric acid; 18.7 g/l NaCl; 1.49 g/l EDTA pH 8.0; 2.5 mg/ml lysozyme (Roche)) was added to each well, the mixture was incubated 1 h at r.t. Subsequently, ⅓ volume of blocking buffer with 9% BSA was added and after an additional 30 min incubation step at r.t., 50 μl of each well was analyzed for binding of sFabs to the target in an ELISA essentially as described for phages, except that detection was performed with an anti-hIgG (Fab-specific) coupled to HRP (1:2500 diluted; Sigma; A 0293).

### Example 2

### Small-Scale Production of Soluble Fab Screening Hits

Unique screening hits were produced in small scale for the initial analysis of their binding to different variants of FGFR-proteins (see example 3). 20-50 ml of LB-medium (supplemented with 0.1 mg/ml ampicillin and 0.1% glucose) were inoculated with a pre-culture of the respective E. coli Top 10 clone, containing a unique Fab sequence cloned into the intial pBIF-vector but lacking the gene III sequence. Production of sFabs was induced by the addition of 0.5 mM IPTG (final concentration) and incubation was continued over night at 30° C. at 250 rpm shaking.

Subsequently, cells were harvested by centrifugation and gently lysed by 1 h incubation at 4° C. in a lysis buffer, containing 20% sucrose (w/v), 30 mM TRIS, 1 mM EDTA, pH 8.0, 1 mg/ml lysozyme (Sigma L-6876) and 2.5 U/ml benzonase (Sigma E1014), followed by the addition of an equal volume of PBS. After that, the cleared supernatant was applied to Dynabeads for His-tag isolation (Invitrogen, 101-03D) and incubated for 2 h at 4° C. on an end-over-end rotator. Subsequently, the matrix was washed 3 times with buffer 1 (50 mM Na-phosphate buffer, pH 7.4, 300 mM NaCl, 5 mM imidazol, 0.01% Tween-20) followed by a single wash step in buffer 2 (PBS containing 0.005% Tween-20). Finally, Fabs were eluted with buffer E (10 mM Na-phosphate buffer, pH 7.4, 300 mM NaCl, 300 mM imidazol) and concentrated in Vivaspin 500 (cut-off 10000; from GE; 28-9322-25) using PBS-buffer, Fabs were analysed for protein content and for purity by SDS-PAGE.

### Example 3

### Cross-Reactivity Profile of Antibodies

Unique screening hits were produced in small scale as described in Example 2 and tested in an ELISA for binding to different FGFR-variants listed in Table 3.

TABLE 3
List of recombinant proteins used in
ELISA for cross-reactivity profiling of binder

| Protein | Origin | Cat. No. (RnD Systems) |
|---|---|---|
| hFGFR2β-Fc (IIIb) | Human | 665-FR |
| mFGFR2β-Fc (IIIb) | Murine | 708-MF |
| hFGFR2α-Fc (IIIb) | Human | 663-FR |
| hFGFR2β-Fc (IIIc) | Human | 684-FR |
| hFGFR1β-Fc (IIIc) | Human | 661-FR |
| hFGFR1β-Fc (IIIb) | Human | 765-FR |
| hFGFR3-Fc (IIIc) | Human | 766-FR |
| hFGFR3-Fc (IIIb) | Human | 1264-FR |
| hFGFR4-Fc | Human | 685-MF |
| mFGFR2β-Fc (IIIc) | Murine | 716-MF |
| mFGFR3-Fc (IIIc) | Murine | 710-MF |
| hTRAIL-Fc | Human | 630-TR |

All variants used were present as Fe-fusion proteins in carrier free preparations. Proteins were biotinylated using an approximately 2-fold molar excess of biotin-LC-NHS (Pierce; Cat. No. 21347) according to manufacturer's instructions and desalted using Zeba desalting columns (Pierce; Cat. No. 89889).

For the ELISA 96-well plates pre-coated with streptavidin (Pierce, 15500) were coated over night at 4° C. with 1 μg/ml biotinylated protein. Wells coated with biotinylated TRAIL-Fc served as a reference. The next day plates were washed 3 times with PBST, treated with blocking reagent, and washed again 3 times with PBST. 100 μl of purified Fabs (1 μg/ml) were added and incubated for 1 h at r.t. After washing 3 times with PBST, an anti-hIgG (Fab-specific) coupled to HRP (1:2500 diluted; Sigma; A 0293) was added and incubated for 1 h at Lt. Color reaction was developed by addition of 50 μl TMB (Invitrogen, 2023) and stopped after 5-15 min by adding 50 μl H₂SO₄ (Merck, 1120801000). Colorimetric reaction was recorded at 450 nM in a plate reader (Tecan). Wells containing TRAIL-Fc were used as background values and the signal to background ratios were calculated as summarized in Table 4.

TABLE 4
Summary of ELISA-data on cross-reactivity of antibodies

| | | | | | | | | | hFGFR3- | | |
|---|---|---|---|---|---|---|---|---|---|---|---|
| | hFGFR2β- | hFGFR2β- | hFGFR2α- | mFGFR2β- | mFGFR2β- | hFGFR1β- | hFGFR1β- | hFGFR3-Fc | Fc | mFGFR3- | hFGFR4- |
| | Fc (IIIb) | Fc (IIIc) | Fc (IIIb) | Fc (IIIb) | Fc (IIIc) | Fc (IIIb) | Fc (IIIc) | (IIIb) | (IIIc) | Fc (IIIc) | Fc |
| M048- | +++ | +++ | +++ | +++ | +++ | 0 | 0 | 0 | 0 | 0 | 0 |
| D01 | | | | | | | | | | | |
| M017- | +++ | +++ | +++ | +++ | +++ | 0 | 0 | 0 | 0 | 0 | 0 |
| B02 | | | | | | | | | | | |
| M021- | +++ | +++ | +++ | +++ | +++ | 0 | 0 | 0 | 0 | 0 | 0 |
| H02 | | | | | | | | | | | |
| M054- | ++ | ++ | +++ | +++ | ++ | 0 | 0 | 0 | 0 | 0 | 0 |
| A05 | | | | | | | | | | | |
| M047- | +++ | +++ | +++ | +++ | ++ | 0 | 0 | 0 | 0 | 0 | 0 |
| D08 | | | | | | | | | | | |
| M054- | + | + | ++ | ++ | + | 0 | 0 | 0 | 0 | 0 | 0 |
| D03 | | | | | | | | | | | |

Signal to background ratios: 0: <2; +: 2-3; ++: 3-5; +++: >5

As shown in Table 4 the antibodies of this invention bind to human and murine FGFR2 independent of alpha and beta as well as IIIb and IIIc splice form. The antibodies of this invention do not bind to FGFR1, FGFR3, and FGFR4 as shown in Table 4.

### Example 4

### Binding of FGFR2 Antibodies to Cell Surface of Cancer Cell Lines

To determine the binding characteristics of the anti-FGFR2 antibodies on mouse, rat and human cancer cell lines, binding was tested by flow cytometry to a panel of cell lines. Adherent cells were washed twice with PBS (without Ca and Mg) and detached by enzyme-free PBS based cell dissociation buffer (Invitrogen). Cells were suspended at approximately 10⁵ cells/well in FACS buffer (PBS without Ca/Mg, Biochrom containing 3% FCS, Biochrom). Cells were centrifuged (250 g, 5 min, 4° C.) and supernatant discarded. Cell were resuspended in dilutions of the antibodies of interest (5 μg/ml in 80 μl if not indicated otherwise) in FACS buffer, and incubated on ice for 1 h. In the following cells were washed once with 100 μl cold FACS buffer and 80 μl secondary antibody diluted at 1:150 (PE goat anti-human IgG, Dianova #109-115-098, or PE Goat Anti-Mouse IgG, Jackson Immuno Research #115-115-164) was added. After incubation for 1 h on ice cells were again washed with cold FACS buffer, resuspended in 100 μl FACS buffer and analyzed by flow cytometry using a FACS-Array (BD Biosciences). Results are calculated as Geo Mean of the detection by the antibody of interest subtracted by background fluorescence as measured by detection with the secondary antibody alone. Values are scored according to the following system:

Geo Mean—Geo Mean of secondary antibody alone >10: +, >100: ++, >1000: +++, 10000: ++++, close to category border in ( ).

### List of Cell Lines Used for Cross-Reactivity Profiling of Antibodies:

| | |
|---|---|
| SNU16 | ATCC-CRL-5974 |
| KATOIII | ATCC-HTB-103 |
| NCI-N87 | CRL-5822 |
| HS746T | NCI-60 Panel, Lot 507285 |
| MFM223 | ECACC-98050130 |
| 4T1 | ATCC-CRL-2539 |
| EMT6 | ATCC-CRL-2755 |
| HEC1b | ATCC-HTB-113 |
| ECC1 | ATCC-CRL-2923 |
| MFE296 | ECACC-98031101 |
| MFE280 | ECACC-98050131 |
| AN3CA | DSMZ-ACC 267 |
| RUCA | |
| SUM-52PE | Asterand, Source Steven Ethier |

As shown in Table 5, all anti FGFR2 antibodies of this invention used at a concentration of 5 μg/ml bind a broad range of tumor cells expressing FGFR2 of murine (4T1, EMT6), rat (RUCA) and human (all other cell lines included in the table) origin.

TABLE 5
Binding of anti FGFR2 antibodies 5 μg/ml to different cell lines by scoring of
FACS analysis

| | Gastric cancer cells | | | | Breast cancer cells | | |
|---|---|---|---|---|---|---|---|
| AB | SNU16 | KATOIII | NCI-N87 | HS746T | MFM223 | 4T1 | EMT6 |
| M017-B02-hIgG1 | +++ | ++++ | + | + | ++++ | ++ | (+) |
| M021-H02-hIgG1 | ++ | ++++ | + | +( +) | +++(+) | ++ | +(+) |
| M048-D01-hIgG1 | ++(+) | ++++ | + | + | +++(+) | ++ | + |
| M054-A05-hIgG1 | ++(+) | ++++ | +++ | +++ | ++++ | +++ | +++(+) |
| M054-D03-hIgG1 | ++ | ++++ | + | ++ | +++(+) | +(+) | ++ |
| M047-D08-hIgG1 | ++(+) | ++++ | + | (+) | ++++ | ++ | + |

| | Endometrial cancer cells | | | | | |
|---|---|---|---|---|---|---|
| AB | HEC1b | ECC1 | MFE296 | MFE280 | AN3CA | RUCA |
| M017-B02-hIgG1 | + | +(+) | +++ | + | ++ | ++ |
| M021-H02-hIgG1 | ++ | − | ++ | + | +(+) | ++ |
| M048-D01-hIgG1 | + | + | +++ | + | ++ | + |
| M054-A05-hIgG1 | +++ | ++ | +++ | ++ | ++ | ++(+) |
| M054-D03-hIgG1 | ++ | + | ++ | + | +(+) | ++ |
| M047-D08-hIgG1 | − | + | +++ | + | ++ | ++ |

(Geo Mean-Geo Mean of secondary antibody alone >10: +, >100: ++, >1000: +++, >10000: ++++, close to category border in ( ))

To determine the EC₅₀ values for binding of antibodies to selected cancer cell lines, cells were stained with FGFR2 antibodies as described above, but with various concentrations of antibodies ranging from 0.1-100 nM. EC₅₀ values were determined using Graph Pad Prism Software and are presented in Table 6. Three antibodies with highest affinity (M017-B02-hIgG1, M048-D01-hIgG1, M047-D08-hIgG1) show subnanomolar to low nanomolar EC₅₀ values in human (SNU-16, MFM223), murine (4T1) and rat (Ruca) cell lines. M021-H02-hIgG1, M054-A05-hIgG1 and M054-D03-hIgG1 show also low nM cellular EC₅₀ values in murine and human cell lines. Thus, all tested antibodies are cross reactive in binding to human, murine and rat cells expressing FGFR2.

TABLE 6
EC₅₀ values of anti FGFR2 antibodies
binding to cell lines of human (SNU16, MFM223),
murine (4T1) and rat (RUCA) origin analyzed by FACS

| | EC₅₀ [nM] | | | |
|---|---|---|---|---|
| AB | SNU16 | MFM223 | 4T1 | Ruca |
| M017-B02-hIgG1 | 0.1 | 0.2 | <0.1 | n.d. |
| M021-1-102-hIgG1 | 89.5 | 16.9 | 20.8 | n.d. |
| M048-D01-hIgG1 | 0.4 | 1.9 | 0.1 | 0.9 |
| M054-A05-hIgG1 | 62.2 | 25.7 | 86.1 | n.d. |
| M054-D03-hIgG1 | 79.2 | 13.0 | 46.6 | n.d. |
| M047-D08-hIgG1 | 0.2 | 6.4 | <0.1 | 1.0 |

(n.d. stands for not determined/measured)

### Example 5

### Epitope Mapping by Pepscan's Chemically Linked Peptides on Scaffolds (CLIPS) Technology

To determine the binding characteristics of the antibodies found, an intensive epitope mapping based on Pepscan's proprietary Chemically Linked Peptides on Scaffolds (CLIPS) technology (Timmerman et al., J. Mol. Recognit. 2007, 20:283-99) was performed. In total 8653 different CLIPS peptides of 15AA and 30AA length covering linear, conformational and discontinuous epitopes on the native human FGFR2 were designed. The peptides were synthesized on peptide arrays. Antibodies of this invention were tested on the peptide arrays in human IgG1 format in an ELISA-based assay. The peptides that gave the highest ELISA values were analyzed to identify shared similar amino acid sequences.

To reconstruct discontinuous epitopes of the target molecule a library of structured peptides was synthesized. This was done using Pepscan's proprietary Chemically Linked Peptides on Scaffolds (CLIPS) technology (Timmerman et al., J. Mol. Recognit. 2007, 20:283-99). CLIPS technology allows to structure peptides into single loops, double-loops, triple loops, sheet-like folds, helix-like folds and combinations thereof. CLIPS templates are coupled to cysteine residues. The side-chains of multiple cysteines in the peptides are coupled to one or two CLIPS templates. For example, a 0.5 mM solution of the T2 CLIPS template 1,3-bis(bromomethyl) benzene was dissolved in ammonium bicarbonate (20 mM, pH 7.9)/acetonitrile (1:1(v/v)). This solution was added onto the peptide arrays. The CLIPS template bound to side-chains of two cysteines as present in the solid-phase bound peptides of the peptide-arrays (455 wells plate with 3 μl wells). The peptide arrays were gently shaken in the solution for 30 to 60 minutes while completely covered in solution. Finally, the peptide arrays were washed extensively with excess of H₂O and sonicated in disrupt-buffer containing 1 percent SDS/0.1 percent beta-mercaptoethanol in PBS (pH 7.2) at 70° C. for 30 minutes, followed by sonication in H₂O for another 45 minutes. The T3 CLIPS carrying peptides were made in a similar way but now with three cysteines.

The binding of antibody to each peptide was tested in a PEPSCAN-based ELISA (Slootstra et al., Molecular Diversity 1996, 1: 87-96). The peptide arrays were pre-incubated with 5% to 100° A-binding buffer (1 hr, 20° C.). The binding buffer was composed of 1% Tween-80, 4% horse-serum, 5% Ovalbumin (w/v) and was diluted with PBS. After washing the peptide arrays were incubated with primary antibody solution (1 to 5 ug/ml) in PBS containing 1% Tween-80 (overnight at 4° C.). After washing, the peptide arrays were incubated with a 1/1000 dilution in 100% binding buffer of an antibody peroxidase conjugate for one hour at 25° C. (anti-human). After washing, the peroxidase substrate 2,2′-azino-di-3-ethylbenzthiazoline sulfonate (ABTS) and 2 microliter/milliliter of 3 percent 1-1202 were added. After one hour, the color development was measured. The color development was quantified with a charge coupled device (CCD)—camera and an image processing system.

### Data Processing

The raw data are optical values obtained by a CCD-camera. The values range from 0 to 3000 mAU, similar to a standard 96-well plate ELISA-reader. The binding values were extracted for analysis. Occasionally, a well contains an air-bubble resulting in a false-positive value, the cards were manually inspected and any values caused by an air-bubble were scored as 0.

All antibodies of this invention bind to the same epitope, which comprises of the N-terminal residues of FGFR2 (¹RPSFSLVEDTTLEPE¹⁵). Analysis of 1257 CLIPS and linear peptides showed consistent high ELISA values for N-terminal peptides.

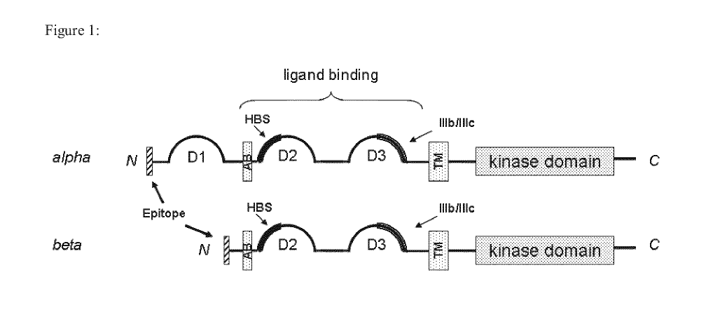

The N-terminal residues (¹RPSFSLVEDTTLEPE¹⁵) are present in all splice variants of human FGFR2 independent of alternative splicing in D3 resulting in IIIb and IIIc isoforms (see FIG. 1). The epitope is also present if domain D1 is spliced out of the full length FGFR2 (SEQ ID NO:61; FGFR2 alpha) resulting in the shorter beta form of FGFR2 (SEQ ID NO:62). In this case the epitope is directly in front of domain D2 (see FIG. 1).

Of special interest is that the N-terminal sequence is conserved in human, mouse, rat and macaca mulatta. This enables broad inter species cross reactivity.

This new epitope is outside the well-known ligand binding site and the heparin binding site (see FIG. 1) and results in novel features of the antibodies of this invention.

### Example 6

### Epitope Fine Mapping by Alanine Scanning of Peptides

To define the binding characteristics of the antibodies of the invention in more detail an Alanine-scanning was performed. As described in example 5, peptides of 15AA and 30 AA lengths were synthesized and each amino acid of the human FGFR2 sequence was replaced for a certain peptide by an Alanine residue. Binding of the antibodies was analyzed as described in Example 5. If the exchange of an amino acid residue for an Alanine results in a significant reduction of the binding signal, this residue was accounted as critical for the binding.

Table 7 shows for the antibodies of this invention the critical residues in the N-terminal part (¹RPSFSLVEDTTLEPE¹⁵) of FGFR2.

TABLE 7
Critical residues in the N-terminal part (¹RPSFSLVEDTTLEPE¹⁵)
of FGFR2 for binding of antibodies of this invention

| | position | | | | | | | | | | | | | | |
|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 |
| | R | P | S | F | S | L | V | E | D | T | T | L | E | P | E |
| M017-B02 | X | X | | X | X | | | | | | | | | | |
| M021-H02 | | | | X | | | | | | | | | | | |
| M047-D08 | X | X | | X | X | | | | | | | | | | |
| M048-D01 | | X | | | | X | | X | | | | | | | |
| M054-D03 | X | X | | X | X | | | | | | | | | | |
| M054-A05 | | | | X | X | | | | | | | | | | |

(Residues being critical for binding are marked by an (X). By changing this residue into an Alanine more than 50% of the ELISA signal is lost)

Antibodies M048-D01 and M021-1-102 are of special interest because they are binding independently of variations at position Ser-5. This enables them to bind in addition to human, mouse, rat and macaca mulatta FGFR2 (SEQ ID NO:63) to rabbit (SEQ ID NO:64), pig (SEQ ID NO:65) and dog (SEQ ID NO:66) FGFR2 making it possible to use even more species for preclinical development.

### Example 7

### Affinity of Antibodies for the N-Terminal Epitope Analyzed by Biacore

To define the binding affinities for the N-terminal peptides characterized as epitopes Biacore surface plasmon resonance experiments were performed.

Binding affinities of anti FGFR2 antibodies were determined by surface plasmon resonance analysis on a Biacore T100 instrument (GE Healthcare Biacore, Inc.). Antibodies as human IgG1 were immobilized onto a CM5 sensor chip through an indirect capturing reagent, anti-human IgG(Fc). Reagents from the “Human Antibody Capture Kit” (BR-1008-39, GE Healthcare Biacore, Inc.) were used as described by the manufacturer. Approximately 5000 RU monoclonal mouse anti-human IgG (Fc) antibody were immobilized per cell. Anti FGFR2 antibodies were injected at a concentration of 5 μg/ml at 141/min for 10 sec. Various concentrations (400 nM, 200 nM, 100 nM, 50 nM, 25 nM, 12.5 nM, 6.25 nM, and 3.12 nM) in HEPES-EP buffer (GE Healthcare Biacore, Inc.) of peptides derived from the first 15 amino acids of FGFR2 of different species (human, mouse, rat, macaca mulatta FGFR2 (SEQ ID NO:63), rabbit (SEQ ID NO:64), pig (SEQ ID NO:65) and dog (SEQ ID NO:66)) were injected over immobilized anti FGFR2 antibodies at a flow rate of 60 μl/min for 3 minutes and the dissociation was allowed for 5 minutes. Sensograms were generated after in-line reference cell correction followed by buffer sample subtraction. The dissociation equilibrium constant (K_{D}) was calculated based on the ratio of association (kₒₙ) and dissociation rated (k_{off}) constants, obtained by fitting sensograms with a first order 1:1 binding model using Biavaluation Software (version 4.0).

M048-D01-hIgG1 and M047-D08-hIgG1, bind with a K_{D} value around 100 nM human, murine, rat and macaca mulatta FGFR2 (for details see Table 8). As supported by the Alanine-scanning M048-D01 showed nearly the same K_{D} value for all peptides derived from several species (see Table 8).

TABLE 8
Monovalent K_{D} values of antibodies M048-D01 and M047-D08 as
measured by Biacore with 15 aminoacid long peptides.

| N-terminal peptide of | | |
|---|---|---|
| species | M048-D01-hIgG1 | M047-D08-hIgG1 |
| human, mouse, rat, macaca | 105 nM | 101 nM |
| mulatta [SEQ ID NO: 63] | | |
| rabbit [SEQ ID NO: 64] | 88 nM | no binding |
| pig [SEQ ID NO: 65] | 70 nM | no binding |
| dog [SEQ ID NO: 66] | 72 nM | no binding |

### Example 8

### Stimulation of P-FGFR2 (Phosphorylated FGFR2) Levels after Short Term Incubation with Anti FGFR2 Antibodies on FGFR2 Overexpressing Cell Lines

To determine the effect of anti FGFR2 antibodies on cellular levels of phosphorylated. FGFR2 (P-FGFR2) after short term incubation, P-FGFR2ELISAS were performed. MFM223 cells were plated at 7000 cells per well in growth medium (MEM Earle (Biochrom; F0315)+10% FCS+2 mM Glutamin) in 96 well plates. 24 h after plating cells were incubated with antibodies (10 μ/ml) for 15 min, followed by two washing steps with PBS and lysis in 100 μl of cold lysis buffer consisting of 50 mM Hepes pH 7.2, 150 mM NaCl, 1 mM MgCl₂, 10 mM Na₄P2O7, 100 mM NaF, 10% Glycerin, 1.5% Triton X-100 and freshly added Complete Protease Inhibitor cocktail (Roche No. 1873580001), 4 mM Na₃VO₄, pH adjusted to 7.4 with NaOH by shaking for 5 min. Samples were shock frozen and stored at −80° C. until analysis. Measurement of P-FGFR2 levels was carried out using a P-FGFR2ELISA kit from R&D Systems according to the manufacturer's instructions. OD was measured at 450 nM (Tecan Spectra, Rainbow) with background correction. Levels of P-FGFR2 were calculated as % of untreated control levels. To control for non-specific effects of the antibody format, parallel samples were incubated with non-cell binding control IgGs of the same isotype.

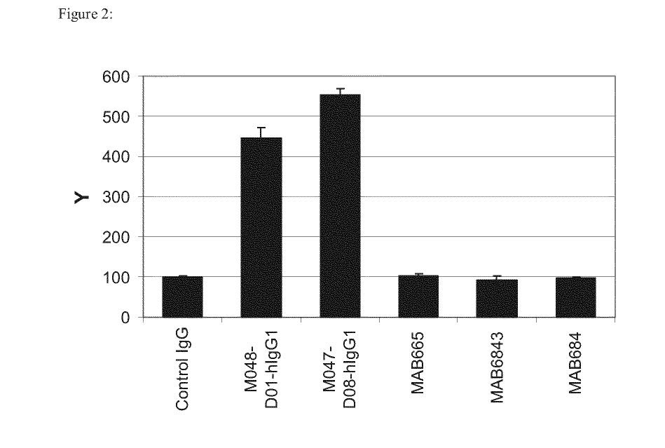

Results are shown in FIG. 2 and indicate a pronounced induction of P-FGFR2 levels by anti FGFR2 antibodies M048-D01-hIgG1 and M047-D08-hIgG1. In contrast neither the control IgG antibody nor anti FGFR2 antibodies commercially available from R&D (MAB665, MAB684, MAB6843) show any significant effect on P-FGFR2 levels after short-term incubation. These results reveal an agonistic effect of anti FGFR2 antibodies described within this invention on FGFR2 after short-term incubation.

### Example 9

### Desensitizing of FGFR2 Overexpressing Cells Against Stimulation of P-FGFR2 by FGF7 after Long-Term Incubation with Anti FGFR2 Antibodies

To determine the effect of anti FGFR2 antibodies on cellular levels of phosphorylated FGFR2 (P-FGFR2) after long term incubation and the effect of antibody treatment on the power of FGF7 to induce FGFR2 phosphorylation, P-FGFR2ELISAS were performed. MFM223 cells were plated at 7000 cells per well in growth medium (MEM Earle (Biochrom; F0315)+10% FCS+2 mM Glutamin) in 96 well plates, 24 h after plating cells were incubated with antibodies (10 μ/ml) for 24 min, followed by incubation in the presence or absence of FGF7 (R&D Systems, 25 ng/ml) for 15 min. Cells were washed twice with PBS and lysed in lysis buffer consisting of (50 mM Hepes pH 7.2, 150 mM NaCl, 1 mM MgCl₂, 10 mM Na₄P₂O₇, 100 mM NaF, 10% Glycerin, 1.5% Triton X-100, freshly added Complete Protease Inhibitor cocktail (Roche No. 1873580001), 4 mM Na₃VO₄, pH adjusted to 7.4 with NaOH) and shaking for 5 min at room temperature. Samples were snap frozen and stored at −80° C. until analysis by the P-FGFR2ELISA from R&D according to the manufacturer's instructions. Optical density was measured at 450 nM (Tecan Spectra, Rainbow) with background correction. Levels of P-FGFR2 were calculated as % of untreated control levels. To control for non-specific effects of the antibody format, parallel samples were incubated with non-cell binding control IgGs of the same isotype.

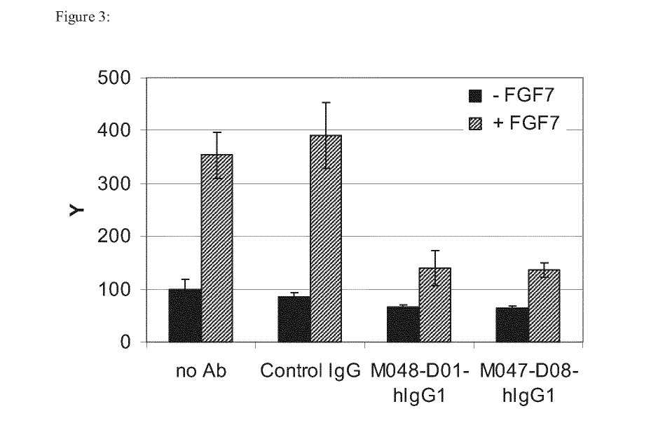

Corresponding results are presented in FIG. 3. In cells treated without antibody treatment as well as in cells treated with isotype control IgG stimulation with FGF7 lead to an about 4 fold increase of P-FGFR2 levels. In contrast, in samples pretreated with anti FGFR2 antibodies for 24 h, FGF7 only induced P-FGFR2 levels by 1.37-1.4 fold.

Taken together these results show that prolonged incubation of cells with anti FGFR2 antibodies of this invention leads to desensitization towards stimulation with FGF7.

### Example 10

### Downregulation of FGFR2 Surface Expression after Incubation of Cell Lines with Anti FGFR2 Antibodies

To analyze FGFR2 surface expression after treatment with anti FGFR2 antibodies FACS analysis was carried out in different cell lines with FGFR2 overexpression (MFM223, SNU16) or FGFR2 mutation (AN3-CA, MFE-296). Adherent cells were washed twice with PBS (without Ca and Mg) and detached by enzyme-free PBS based cell dissociation buffer (Invitrogen). Cells were suspended at 0.5*10⁵ cells/well in 80 μl growth medium (MFM223, MFE-296: MEM Earle (Biochrom; F0315)+10% FCS+2 mM Glutamin, SNU-16: RPMI 1640 (Biochrom, FG1215)+10% FBS; AN3-CA:MEM Earle (Biochrom; FG0325)+10% FCS+1 mM Sodiumpyruvate+1×NEA: non essential amino acids Biochrom K0293). 20 μl of 5 fold concentrated antibody dilution was added (final concentration of 10 μg/ml) and incubated for 4.5 h at 37° C. After the end of the incubation time cells were washed once with 100 μl FACS buffer, stained with detection antibody (at 5 μg/ml, mouse anti-FGFR2 for hIgGs, human anti-FGFR2 for mIgGs) for 45 min at 4° C., followed by an additional wash with 100 μl FACS buffer. PE-Stained secondary antibody (PE goat anti-human IgG, Dianova #109-115-098, or PE Goat Anti-Mouse IgG, Jackson Immuno Research #115-115-164, 1:150 diluted) was added in 80 μl volume, incubated for 45 min at 4° C. and after an additional wash with FACS buffer cells analyzed by flow cytometry using a FACS array (BD Biosciences). In control experiments antibody competition for overlapping epitopes was excluded by parallel incubation with the antibody of interest and the corresponding detection antibody. Geo-Means measured after staining with secondary antibodies alone were subtracted from Geo-Means from peaks detected after staining with anti FGFR2 antibodies. Results are calculated as % of Control cells that were incubated for 4.5 h without the presence of antibodies.

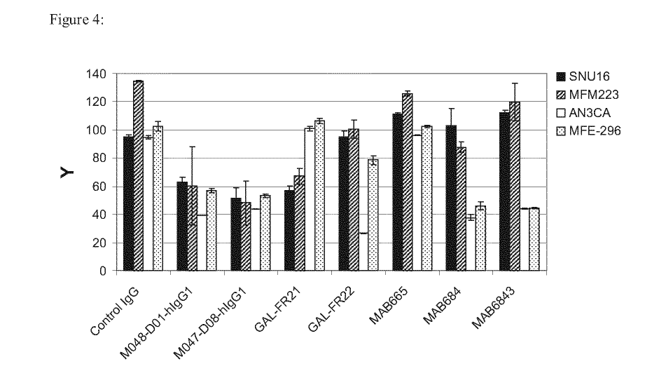

Results are depicted in FIG. 4. Incubation of cells with control IgG leads to no decrease of FGFR2 surface expression, whereas anti FGFR2 antibodies M048-D01-hIgG1 and M047-D08-hIgG1 downregulated FGFR2 surface levels significantly by 39-60% in all 4 cell lines independent of FGFR2 overexpression or mutation. In contrast no other anti FGFR2 antibody either commercially available from R&D (MAB665, MAB684, MAB6843) or described elsewhere for example (GAL-FR21, GAL-FR22; WO2010/054265 and Zhao et al. (Clin Cancer Res. 2010, 16:5750-5758)) showed FGFR2 surface downregulation in all 4 cell lines independent of FGFR2 overexpression or mutations. GAL-FR21 downregulated FGFR2 surface levels in cell lines with FGFR2 amplification (SNU16 and MFM223), but had no impact on cell lines with FGFR2 mutation. GAL-FR22 reached 73 and 21% downregulation of FGFR2 surface expression in FGFR2 mutated cell lines (AN3-CA and MFE-296 respectively), but had no significant impact on surface FGFR2 levels in SNU16 and MFM223 cells, MAB684 and MAB6843 again induced around 60% reduction of FGFR2 surface levels in FGFR2 mutated cell lines without major effects on FGFR2 overexpressing cell lines. Finally, MAB665 did not show any impact on FGFR2 surface levels at all.

To summarize, anti FGFR2 antibodies M048-D01-hIgG1 and M047-D08-hIgG1 are the only anti FGFR2 antibodies inducing FGFR2 surface downregulation in cancer cell lines independent of FGFR2 overexpression or mutation.

### Example 11

### Donwregulation of Total FGFR2 Levels after Long-Term Incubation of Cancer Cells with Anti FGFR2 Antibodies

To analyze whether FGFR2 surface downregulation induced by anti FGFR2 antibodies leads to long-term decrease in total FGFR2 levels, total protein levels of FGFR2 were analyzed by FGFR2ELISA. SNU16 cells were plated at 5000 cells/well in 96 well plates in growth medium (RPMI 1640 (Biochrome, FG1215)+10% FBS). 2 h later cells were incubated with anti FGFR2 antibodies at various concentrations as indicated or corresponding isotype control IgG. 96 h after start of incubation with the antibodies cells were centrifuged for 5 min at 300 g at room temperature, washed twice in PBS and lysed by addition of 100 μl lysis buffer (50 mM Hepes pH 7.2, 150 mM NaCl, 1 mM MgCl₂, 10 mM Na₄P₂O₇, 100 mM NaF, 10% Glycerin, 1.5% Triton X-100, freshly added Complete Protease Inhibitor cocktail (Roche No. 1873580001), 4 mM Na₃VO₄, pH adjusted to 7.4 with NaOH) and shaking for 5 min at room temperature. Samples were snap frozen and stored at −80° C. until analysis using the Total-FGFR2-ELISA Ki (R&D Systems) according to the manufacturer's instructions. Optical density was measures at 450 nM (Tecan Spectra, Rainbow) together with background correction. To calculate absolute levels of total FGFR2 standard curve using isolated FGFR2 protein was applied according to the manufacturer's recommendations (R&D Systems). Results are depicted as % of FGFR2 levels measured in control cells that were incubated for 96 h in the absence of antibody.

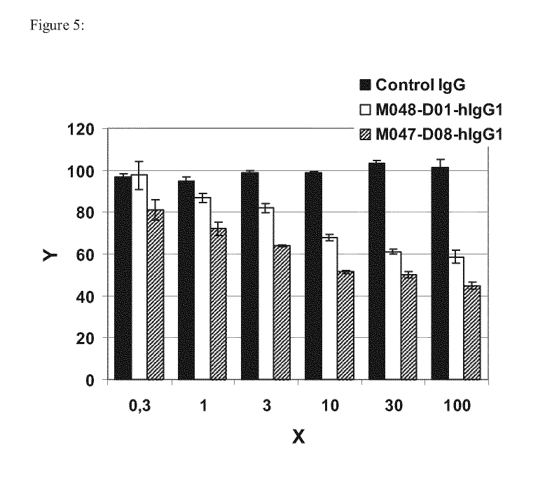

Results are presented in FIG. 5. Incubation with anti FGFR2 antibodies of this invention for 96 h leads to a reduction of total FGFR2 levels by 41-55%. Half maximal reduction is reached at doses of 3 μg/ml of the anti FGFR2 antibodies. In contrast, incubation with isotype control antibody has no effect on total FGFR2 levels.

Taken together, these results indicate that anti FGFR2 antibodies M048-D01-hIgG1 and M047-D08-hIgG1 do not only lead to a short term decrease in surface FGFR2 levels but also a long term reduction of total FGFR2 levels.

### Example 12

### Internalization of Anti FGFR2 Antibodies into Cells

Anti FGFR2 antibodies of this invention were analyzed for their capability to internalize after binding to the FGFR2 antigen.

To visualize this process the FGFR2 specific antibodies M048-D01-hIgG1 and M047-D08-hIgG1 and an isotype control antibody were selected. The antibodies were conjugated in the presence of a two molar excess of CypHer 5E mono NHS ester (batch 357392, GE Healthcare) at pH 8.3. After the conjugation the reaction mixture was dialyzed (slide-A-Lyser Dialysis Cassettes MWCD 10 kD, Fa. Pierce) overnight at 4° C. to eliminate excess dye and adjusting the pH-value. Afterwards the protein solution was concentrated (VIVASPIN 500, Fa Sartorius stedim biotec). In addition to the pH-dependent fluorescent dye CypHer5E the ph-independent dye Alexa 488 was used. The dye load of the antibody was determined with a spectrophotometer (Fa. NanoDrop). The dye load of M048-D01-hIgG1 and M047-D08-hIgG1 and the isotype control (M014) were in a similar range. The affinity of the labeled antibodies was tested in a cell binding-assay to ensure that labeling did not alter the binding to FGFR2. These labeled antibodies were used in the following internalization assays. Prior to treatment cells (2×10⁴/well) were seeded in 100 μl medium in a 96-MTP (fat, black, clear bottom No 4308776, Fa. Applied Biosystems). After 18 h incubation at 37° C./5% CO2 medium was changed and labeled anti FGFR2 antibodies M048-D01-hIgG1 and M047-D08-hIgG1 were added in various concentrations (10, 5, 2.5, 1, 0.3, 0.1 μg/ml). The identical treatment was carried out with the isotope control antibody (negative control). The incubation time was chosen to be 0, 5 h, 1 h, 2 h, 3 h, 6 h and 24 h. The fluorescence measurement was performed with the InCellAnalyzer 1000 (Fa. GE Healthcare). Granule counts and total fluorescence intensity were measured in a kinetic fashion.

A highly specific and significant internalization of M048-D01-hNG1 and M047-D08-hIgG1 was observed in endoenous FGFR2 expressing cancer cell lines SNU16 (gastric cancer) and SUM52PE (breast cancer).

This internalization was target dependent as uptake could only be demonstrated using the anti FGFR2 antibodies while no internalization was observed with the isotype controls. During the first 6 h the anti FGFR2 antibodies showed a 20-40-fold increase of antibody internalization compared to isotype controls. Isotype control showed a minor internalization after a long exposure (>24 h).

Internalization of anti FGFR2 antibodies labeled with Alexa 488 upon binding reveals that more than 50% of internalized antibodies seem to follow the endocytotic pathway.

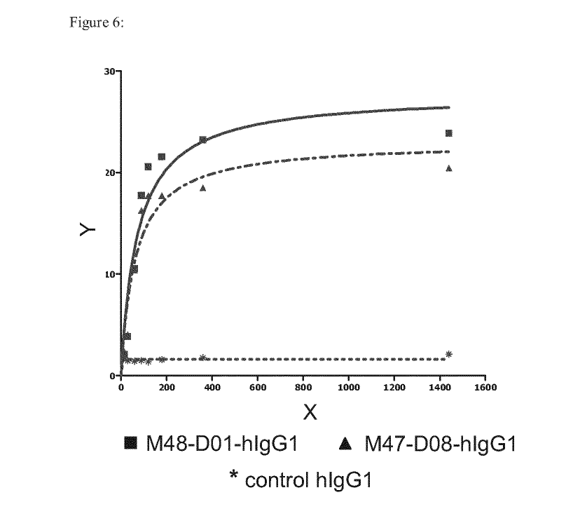

In FIG. 6 a microscopic evaluation of the time course of specific internalization of M048-D01-hIgG1 and M047-D08-hIgG1 upon binding to endogenous FGFR2 expressing cells is shown. Internalization of antibodies (2.5 μg/ml) was investigated on breast cancer cell line SUM 52PE. Granule counts were measured in a kinetic fashion. Rapid internalization could be observed for M048-D01-hIgG1 and M047-D08-hIgG1, whereas the isotype control hIgG1 does not internalize.

A more detailed evaluation of the trafficking pathway was performed with co-staining of small G-proteins. Rab GTPases regulate many steps of membrane traffic, including vesicle formation, vesicle movement along actin and tubulins networks, and membrane fusion. To distinguish between different pathways two Rab proteins were selected for staining—Rab7, which is expressed in late endosomes and lysosomes and Rab 11, which is expressed in early and recycling endosomes. After a 6 h internalization of labeled antibodies the cells were fixed and permeabilized with methanol prior to staining with Rab 7- and Rab 11-antibodies. The results are shown in FIG. 7.

M048-D01-hIgG1 and M047-D08-hIgG1 show a significant co-staining with Rab 7, whereas the co-staining with Rab 11 is only minor. These results indicate that after internalization of FGFR2 the complex enters the endosomal-lysosomal pathway.

The staining pattern for other described antibodies like GAL-FR21 and GAL-FR22 (WO2010/054265 and Zhao et al. (Clin Cancer Res. 2010, 16: 5750-5758)) looks completely different. Here almost no staining could be detected with Rab7, but a major co-staining was achieved with Rab11. This indicates that these antibodies internalize after binding to the FGFR2 receptor and favor the recycling pathway

### Example 13

### Test of Anti FGFR2 Antibodies of this Invention in Experimental Tumors in Mouse Model

In vivo efficacy of the anti FGFR2 antibodies of this invention was for example tested via subcuteanous xenogeneic or allogeneic tumor models. The expert knows prior art methods in order to proof for efficacy of the innovative antibodies. For example, mice were therefore subcutaneously inoculated with tumor cells, which express the target FGFR2. Afterwards, tumor-bearing mice were either treated with FGFR2-targeting antibodies of this invention, non-binding isotype control or phosphate-buffered saline (PBS). Application of antibodies was carried out intraperitoneally or intravenously two times weekly. In order to test for additive anti-tumor efficacy, the FGFR2Abs of this invention were combined with common standard of cares and compared to the single agent efficacies. Tumor growth was monitored by frequent measurement of tumor area via a caliper. After tumor growth and treatment for some weeks, tumors were harvested and tumor weights or tumor sizes (tumor area calculated by the formula length×width) of animals treated with the anti FGFR2 antibodies of this invention were compared to those treated with PBS or isotype control antibodies. Mice treated with the anti FGFR2 antibodies of this invention displayed significantly smaller tumors.

Human or murine tumor cells that express FGFR2 were subcutaneously inoculated onto the flank of immunocompromised mice, for example Nude- or SCID-mice. Per mouse 0.25-10 million cells were detached from cell culture flasks, centrifuged and suspended in 100 μl PBS, 50% medium/50% Matrigel, or 100% Matrigel, respectively. Cells were than inoculated subcutaneously beneath the skin onto the flank of mice. In case of patient-derived tumor models, tumors harvested from gastric cancer patients were subcutaneously passaged on immunocompromised mice. For testing efficacy of the anti FGFR2 antibodies, tumor pieces of a defined size (2×2 mm) were subcutaneously transplanted onto the flank of mice. Within a couple of days a tumor was established. Treatment started earliest if tumors reached a size of 20 mm² (cell line-derived tumors) or 100 mm³ (patient-derived tumors), whereby tumor area (mm²) was calculated by the formula length×width and tumor volume (mm³) by the formula length×width²/2. Treatment with the antibodies was performed either intraperitoneally or intravenously via tail vein injection. Antibodies were either solved in PBS or 50 mM Na-acetat, 150 mM NaCl. Antibodies were applied in a volume of 10 ml/kg. Treatment schedule was based on the pharmacokinetic behavior of the antibody. As standard, antibodies were applied twice weekly (alternating every third and fourth day). As standard, treatment was performed until control group reaches the maximal possible tumor size. Alternatively, treatment was stopped earlier. As standard, 8 mice per treatment group were used. Number of mice per treatment group can be increased, if higher variations in tumor growth were expected. In parallel to the treatment groups, a control group was treated with PBS following the same treatment schedule. During the study, tumor area was frequently assessed by measuring length and width of tumors using a caliper. At study end, tumors were harvested and weighed. Ratio of mean tumor weights of the antibody-treated groups (T) and mean tumor weights of the control (C) was stated as T/C. If treatment and control groups were terminated at different time points or tumor weight could not been used as read-out since tumors became necrotic, T/C ratios were calculated based on tumor area of the last common measurement time point.

2 Mio human gastric cancer SNU-16 cells in 50% medium/50% Matrigel were subcutaneously inoculated onto the flank of female nodSCID mice. Intraperitoneal treatment with the anti-FGFR2 antibodies started when tumors reach a mean size of 20-30 mm² and was continued twice weekly until study end. If tumors of control group reached the maximal acceptable size, study was terminated and tumors are harvested and weighed.

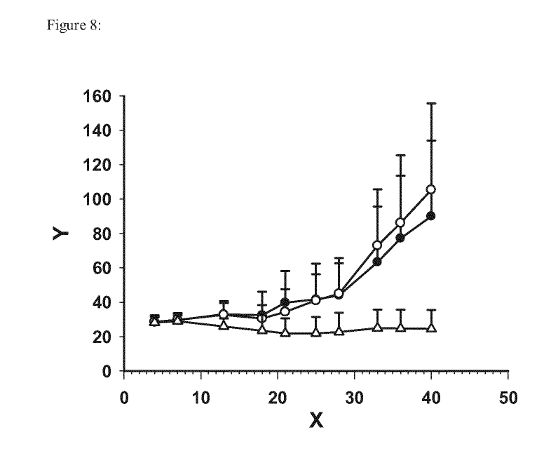

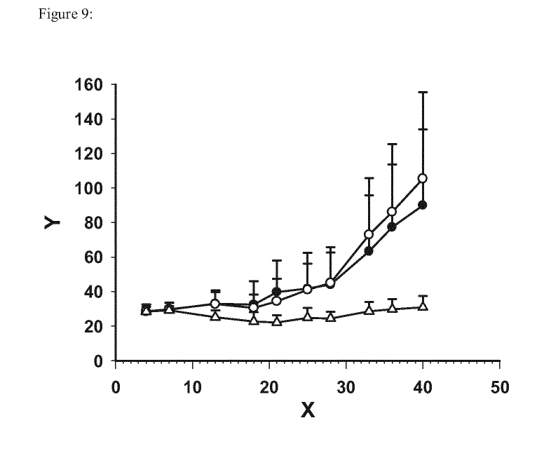

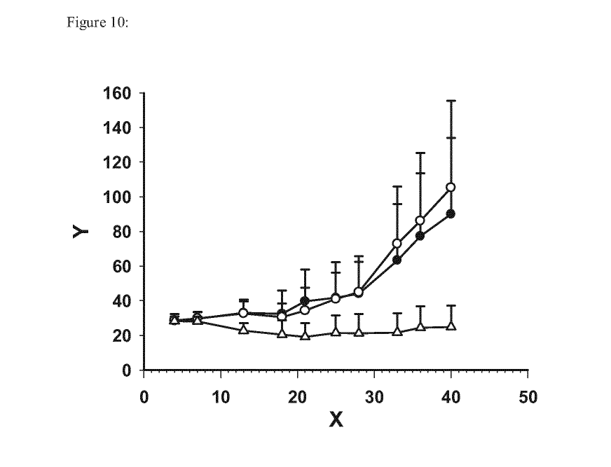

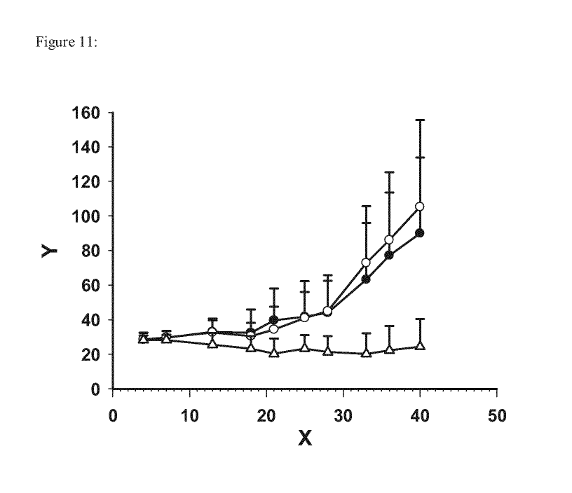

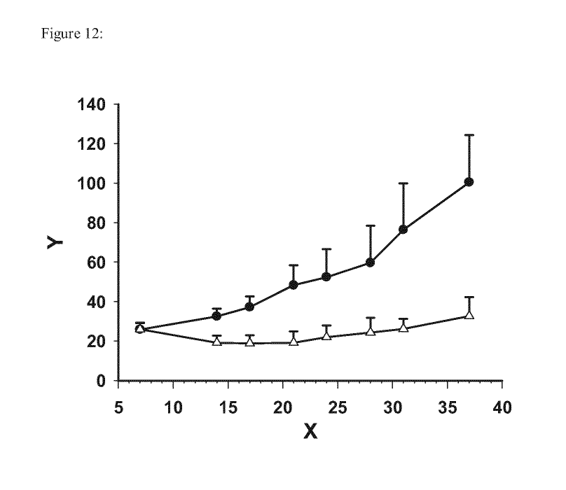

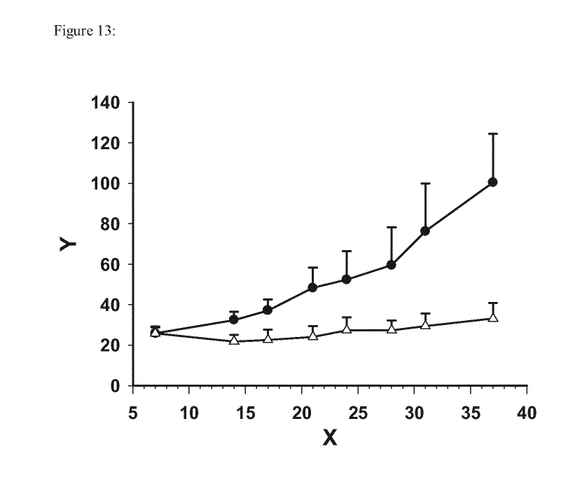

All tested anti FGFR2 antibodies of this invention reduced significantly tumor growth as compared to control. Treatment with a dose of 2 mg/kg of M017-B02-hIgG1, M021-H02-hIgG1, M048-D01-hIgG1, M054-A05-hIgG1, M054-D03-hIgG1 and M047-D08-hIgG1 resulted in T/Cs of 0.19, 0.22, 0.17, 0.19, 0.21 and 0.22, respectively (see FIGS. 8 to 13).

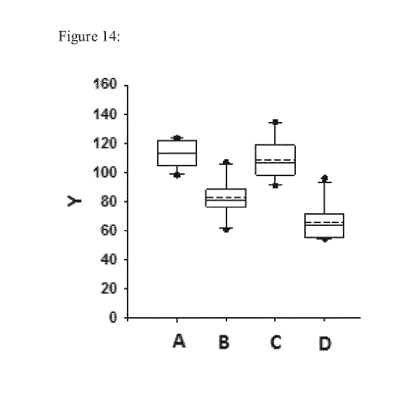

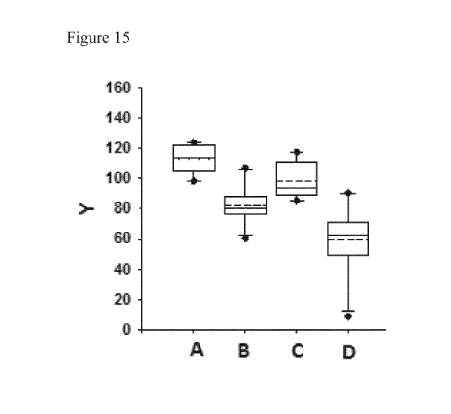

2.5×10⁵ murine 4T1 breast cancer cells were subcutaneously inoculated in 100% PBS onto the flank of NMRI nu/nu mice. Immunocompromised instead of syngeneic mice were chosen in order to avoid the development of neutralizing antibodies against the human IgG protein. Treatment of tumors started at the time point at which tumors have reached a mean size of 24 mm². In order to test for possible additive anti-tumor efficacy of M048-D01-hIgG1 mice were either treated with M048-D01-hIgG1, Lapatinib or Taxol, respectively, alone and in combination with M048-D01-hIgG1 and Taxol or Lapatinib. As control, mice were treated with PBS alone, Treatment with M048-D01-hIgG1 was carried out twice weekly intravenously (i.v.), Lapatinib once daily per os (p.o.) and Taxol once weekly intravenously. All treatments were performed until end of the study. Since tumors became necrotic at the end of the study, tumor area at day 13 after tumor cell inoculation was used to determine anti-tumor efficacy. This study revealed that combination of M048-D01-hIgG1 with either Lapatininb or Taxol achieved additive anti-tumor efficacy: Monotherapy with Lapatinib and Taxol, respectively did not significantly changed growth of tumors as compared to the vehicle control, while M048-D01-hIgG1 alone resulted in significant reduction as compared to vehicle with a T/C of 0.73. Combination with Lapatinib and Taxol reduced this T/C down to 0.58 and 0.52, both statistically significant versus both monotherapies (see FIGS. 14 and 15).

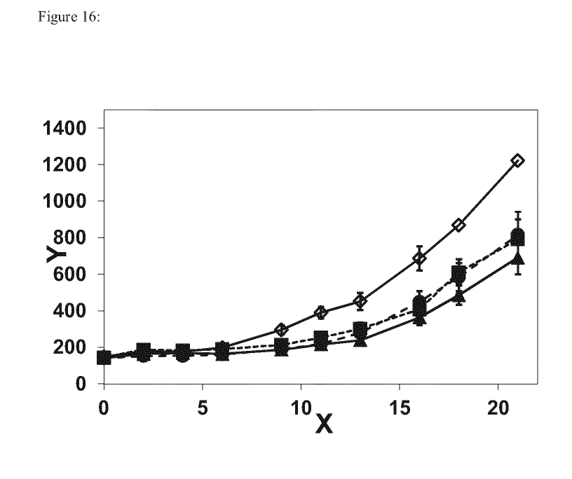

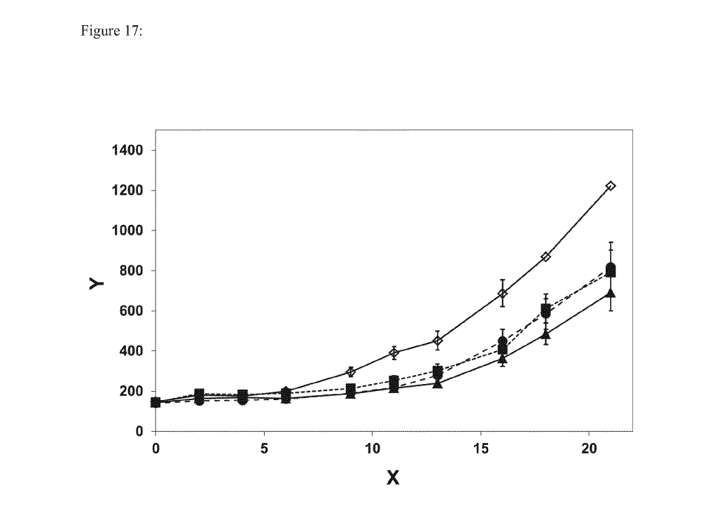

2×2 mm pieces of originally patient-derived gastric tumors, GC10-0608 and GC12-0811 (Prof. Huynh Hung, National University of Singapore (NUS)), passaged on immunocompromised mice, were subcutaneously transplanted onto female immunocompromised naïve mice. Tumor size was assessed frequently using a caliper measuring the tumor in two dimensions and tumor volume was calculated by the formula length×width²/2. Treatment with different doses of M048-D01-hIgG1 was started at the time point at which tumors reached a mean size of approximately 100 mm³. Treatment was performed intravenously twice weekly with doses of 5, 2 and 1 mg/kg M048-D01-hIgG1. In a tumor model with high FGFR2 protein expression (GC10-0608), all three doses resulted in significant reduction of tumor growth resulting in T/C values based on final tumor weight of 0.55, 0.60 and 0.41 (see FIG. 16). In a model with markedly lower FGFR2 protein expression (GC12-0811), 5 and 2 mg/kg of M048-D01-hIgG1 resulted in significant reduction of tumor weight resulting in T/Cs 0.70 and 0.67 (see FIG. 17). In accordance with the lower FGFR2 expression, 1 mg/kg of M048-D01-hIgG1 did not result in significant reduction of final tumor weight. For the treatment of two other tumor models, the breast cancer model MFM223 and the colorectal cancer model NCI-H1716 (ATCC-CCL-251) we have not found an appropriate application scheme to reduce tumor growth significantly.

### Example 14

### Downregulation of P-FGFR and Total FGFR2 Levels in Xenograft Tumors after Treatment with Anti FGFR2 Antibodies

To analyze whether the observed downregulation of total FGFR2 levels and concurrent reduction in P-FGFR2 is also seen in xenograft tumors in vivo, SNU-16 tumors after treatment with anti FGFR2 antibodies were analyzed by Western Blot. Tumors were collected at the end of a xenograft experiment in NOD/SCID mice, treated with anti FGFR2 antibodies 2 mg/kg i.p. twice weekly (see Example 13 for details). Tumors were taken 24 h after the last injection of the antibodies, snap frozen in liquid nitrogen and stored at −80° C. until analysis. Prior to Western Blot analysis frozen tumors were cut in slices of around 5 mm diameter and each slice deposited in a 2 ml Eppendorf tube together with a precooled 5 mm steel bull (Qiagen) and 500 μl lysis buffer (50 mM Hepes pH 7.2, 150 mM NaCl, 1 mM MgCl₂, 10 mM Na₄P₂O₇, 100 mM NaF, 10% Glycerin, 1.5% Triton X-100, freshly added Complete Protease Inhibitor cocktail (Roche No. 1873580001), 4 mM Na₃VO₄, pH adjusted to 7.4 with NaOH). Samples were lysed for 3 min at 300 Hz in a Tissuelyzer (Qiagen) followed by incubation on ice for 30 min. In the following, samples were centrifuged for 10 min at 13000 rpm at 4° C. in a Micro-centrifuge (Eppendorf) and supernatants from slices coming from one original tumor pooled back together. Protein levels in the tumor lysates were determined by using the BCA protein assay kit (Novagen, lysates 1:50 diluted in H₂0). Samples were diluted to a final concentration of 5 mg/ml and 50 μl of sample were mixed with 7.7 μl of (10*) Sample Reducing agent and 19.2 μl (4*) NuPAGE Sample Buffer (Invitrogen). Samples corresponding to 115 μg of protein were applied to NuPage 4-12% SDS page gels from Invitrogen and run for 2 h45 min at 120V. Blotting was carried out by an iBlot system (Invitrogen) according to the manufacturer's recommendations. Membranes were blocked for 2 h at room temperature in 5% BLOT QuickBlocker in PBST (Invitrogen), followed by incubation with primary antibodies over night at 4° C. Primary antibodies were as follows: P-FGFR: #AF3285, R&D Systems, 0.5 μg/m; total FGFR2: M017-B02-hIgG1, 4 μg/ml in 3% BLOT QuickBlocker in PBST. On the next day membranes were washed three times in PBST, followed by incubation with secondary antibodies (Peroxidase-conjugated AffiniPure Goat Anti-Rabbit IgG (H+L) (Jackson ImmunoResearch #111-035-003 or Peroxidase-conjugated AffiniPure Goat Anti-Human IgG+IgM (H+L) (Jackson ImmunoResearch #109-035-127, 1:10000 in 3% BLOT QuickBlocker/PBST) for 2 h at room temperature. Subsequently, membranes were washed four times for 10 min with PBST and signals were detected by chemoluminescence after incubation with ECL reagent. To detect the loading control, membranes were stripped with stripping solution strong (1:10 in Milipore-H2O) for 15 min shaking at room temperature, followed by blocking and detection with Anti-Actin antibody #A2066 (Sigma) 1:1000 in 3% QuickBlocker/PBST.

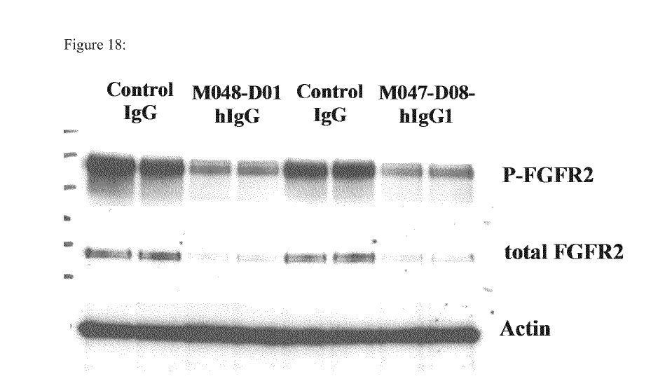

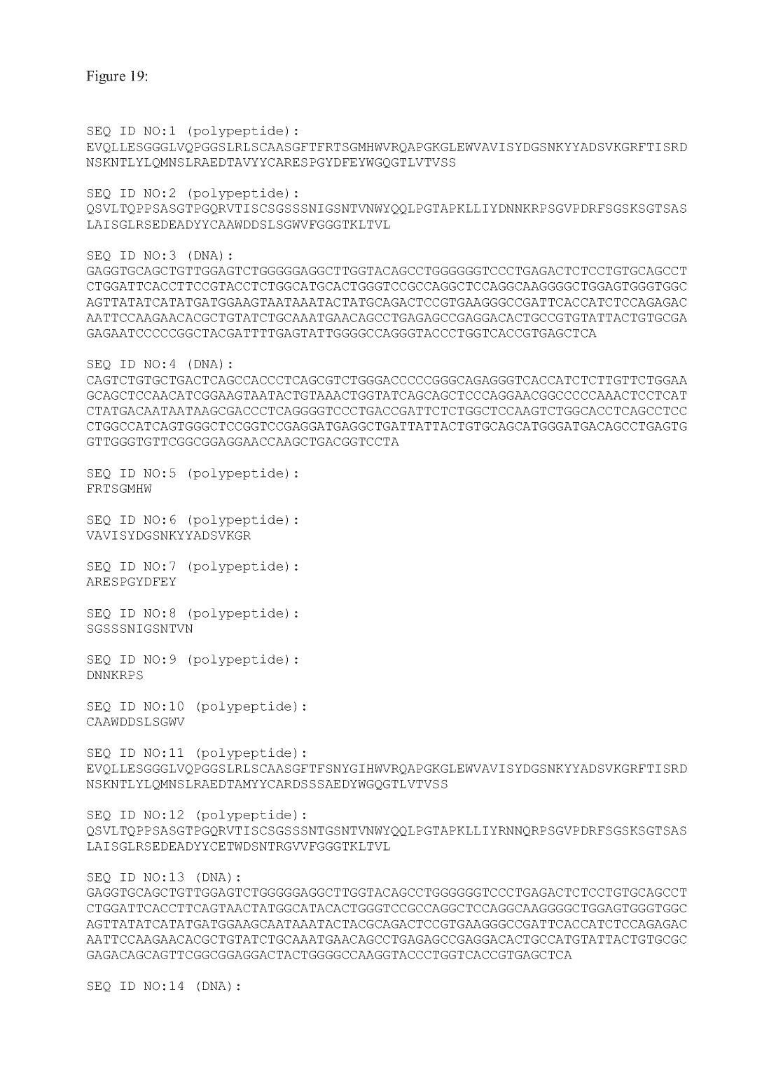

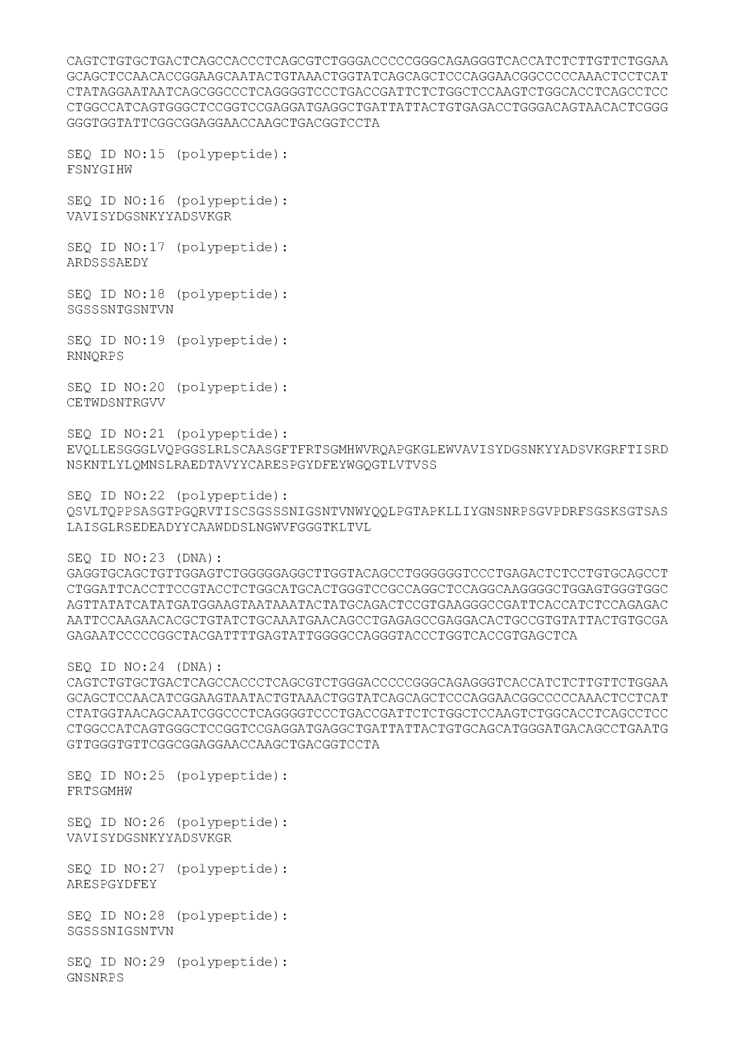

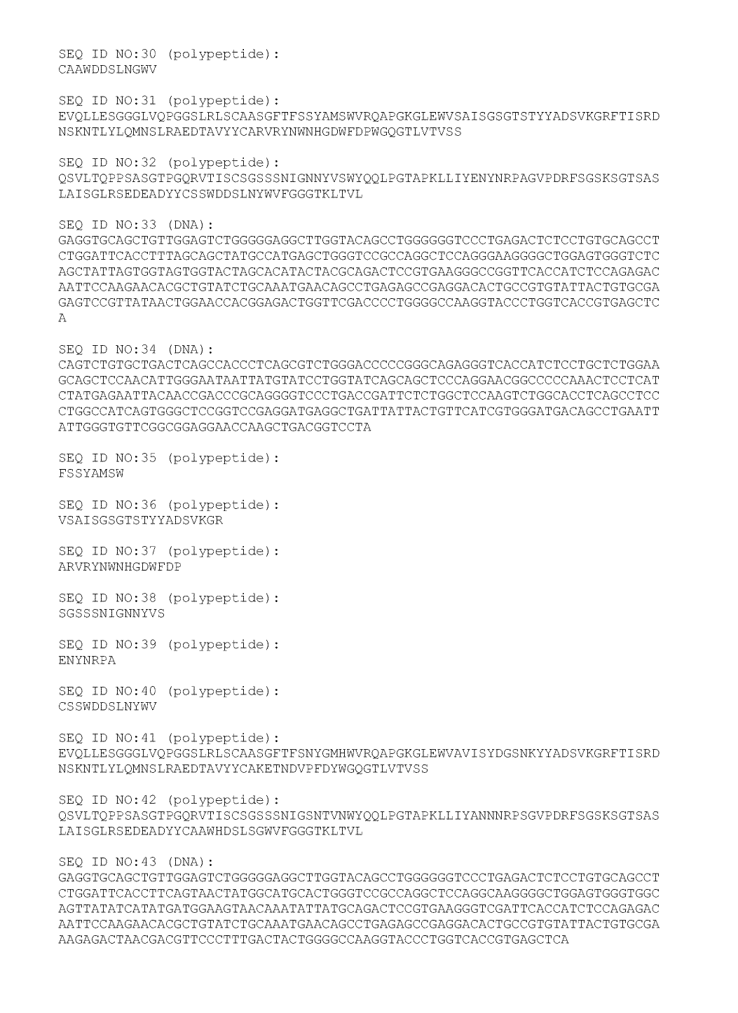

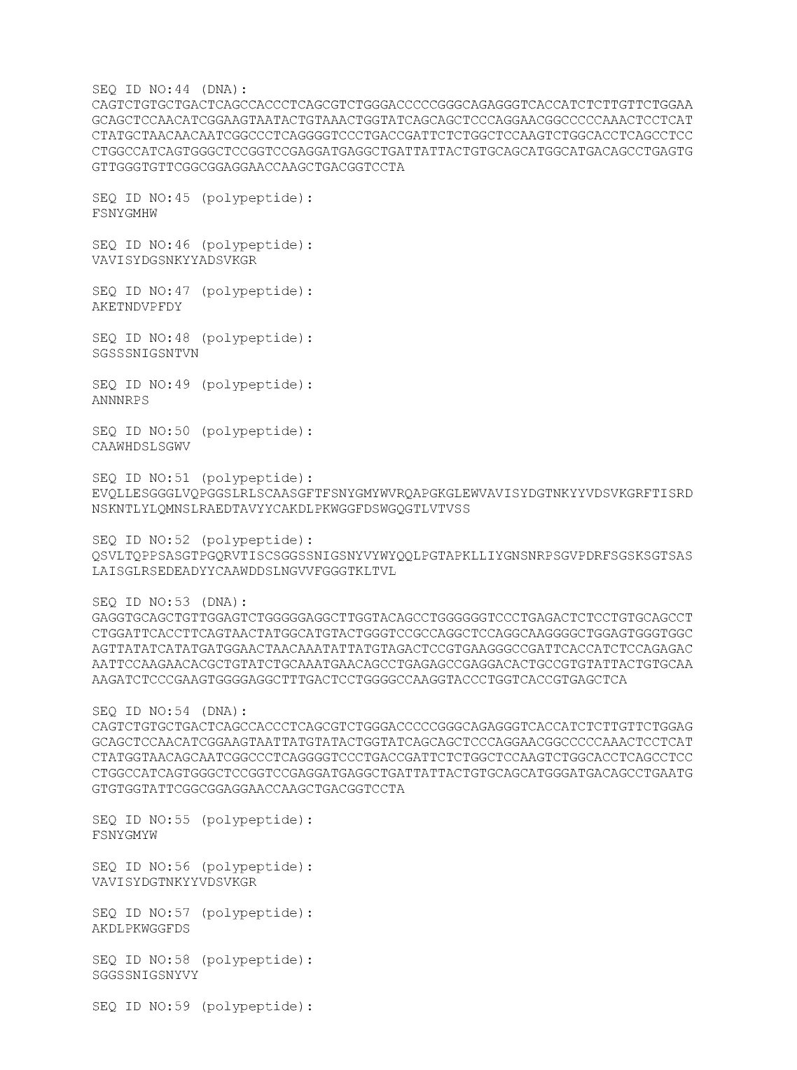

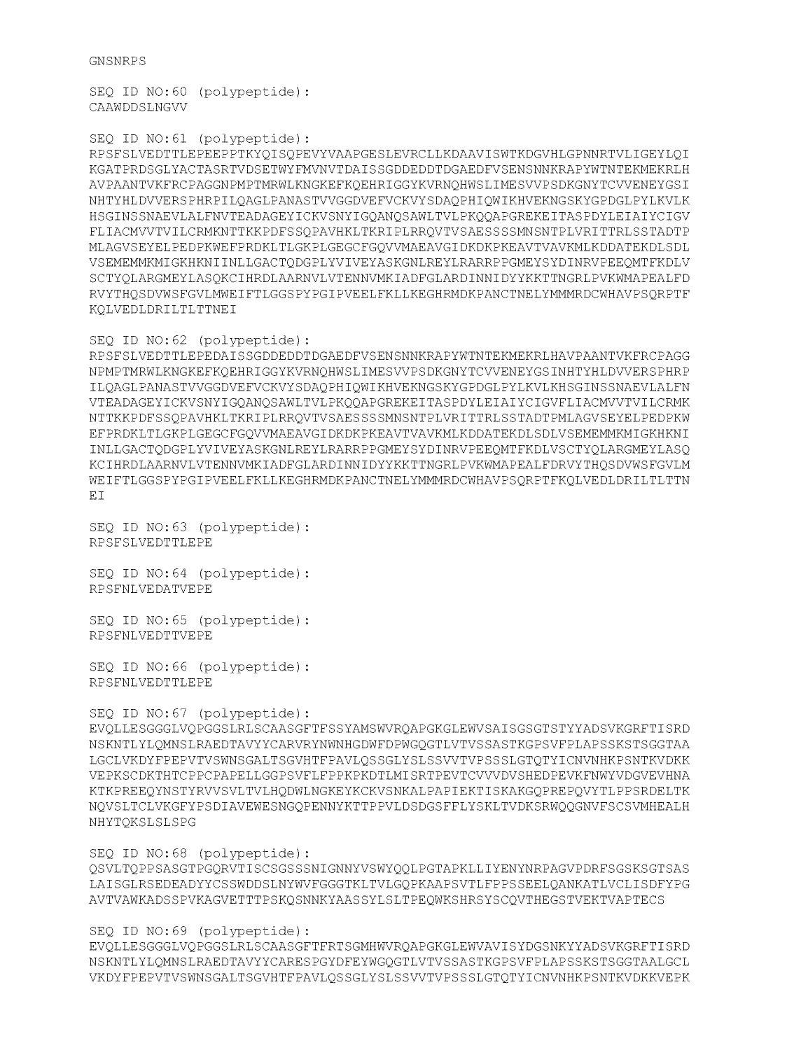

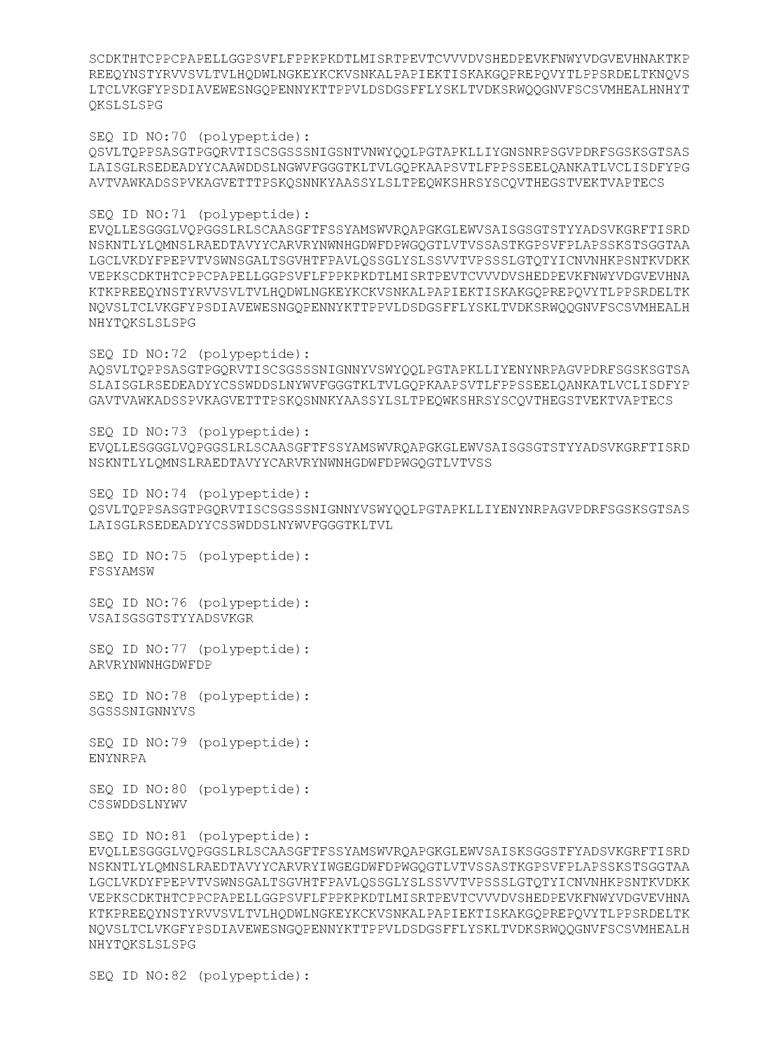

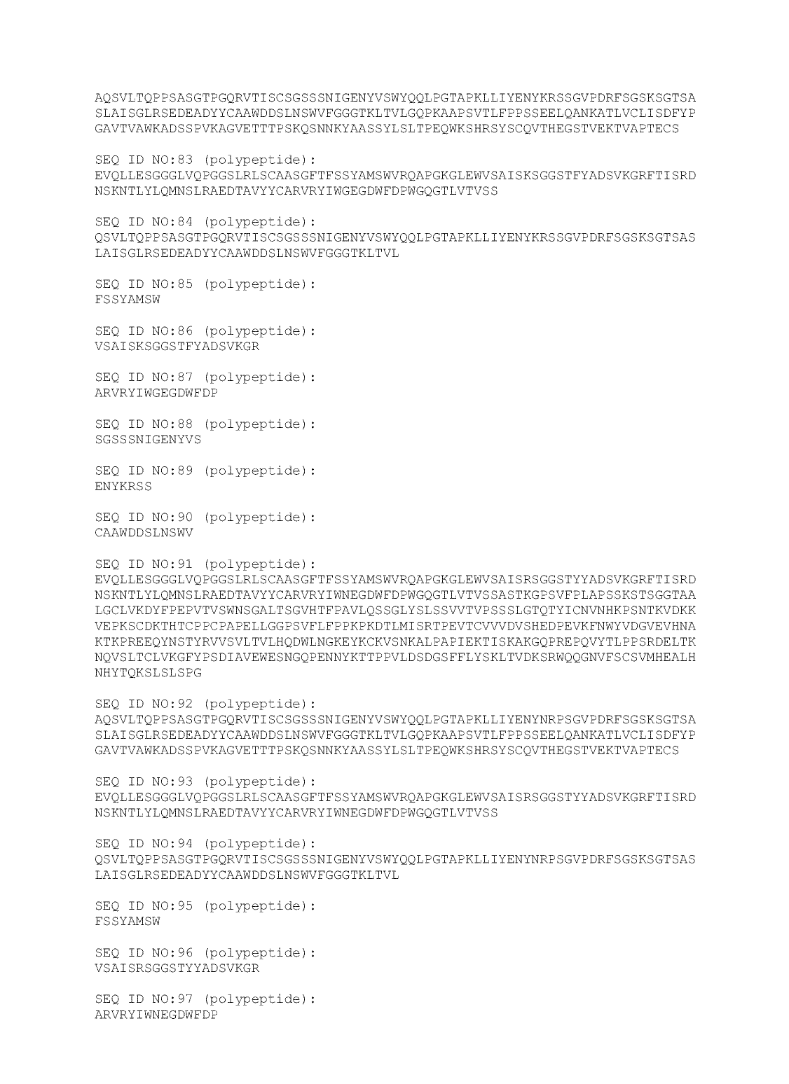

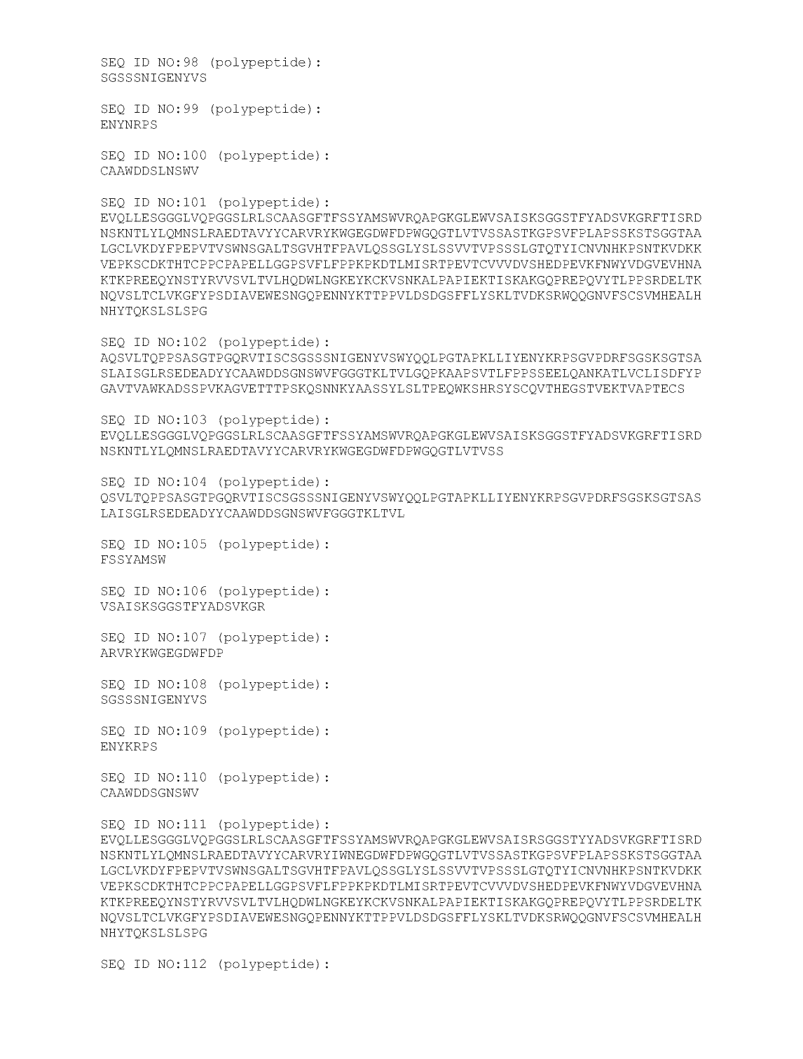

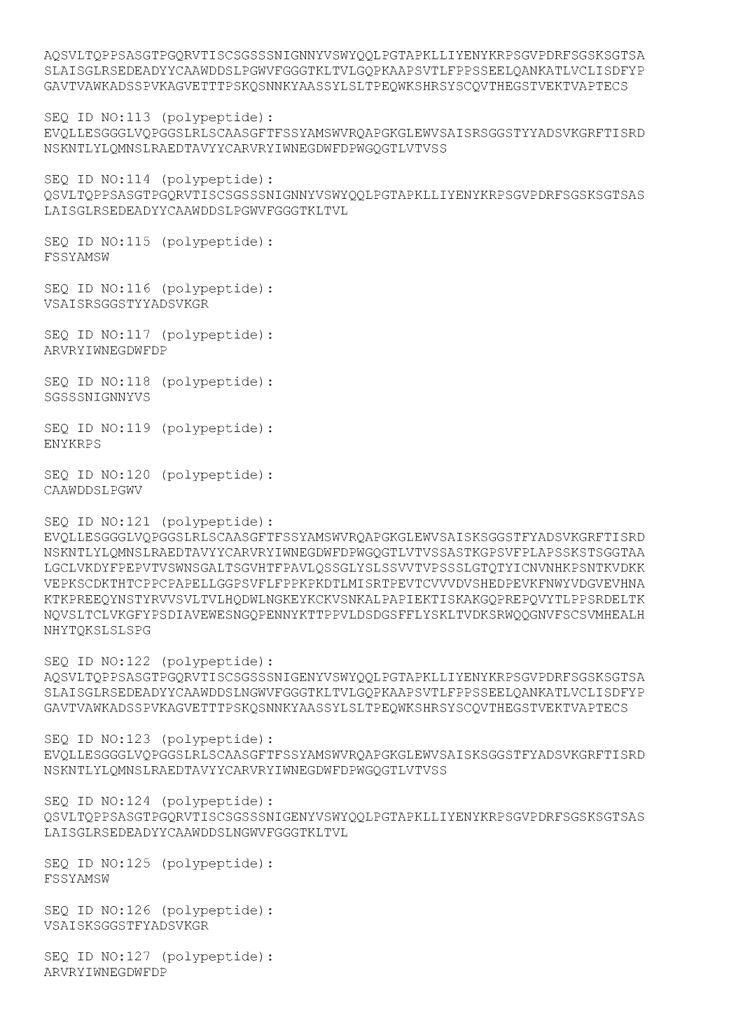

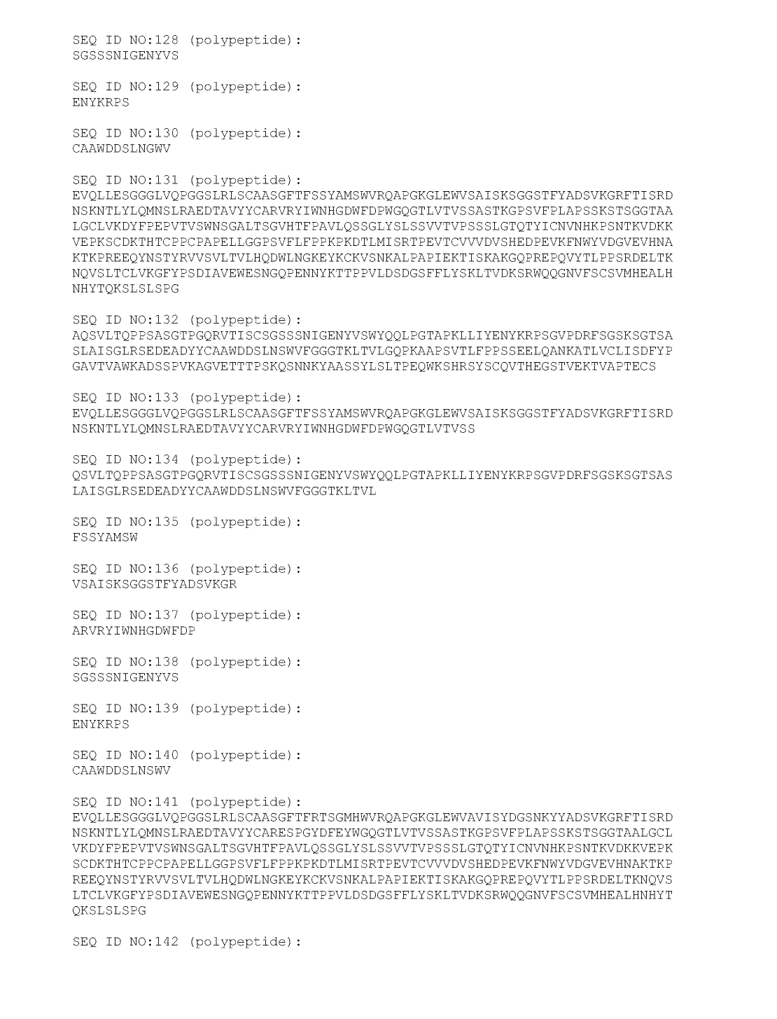

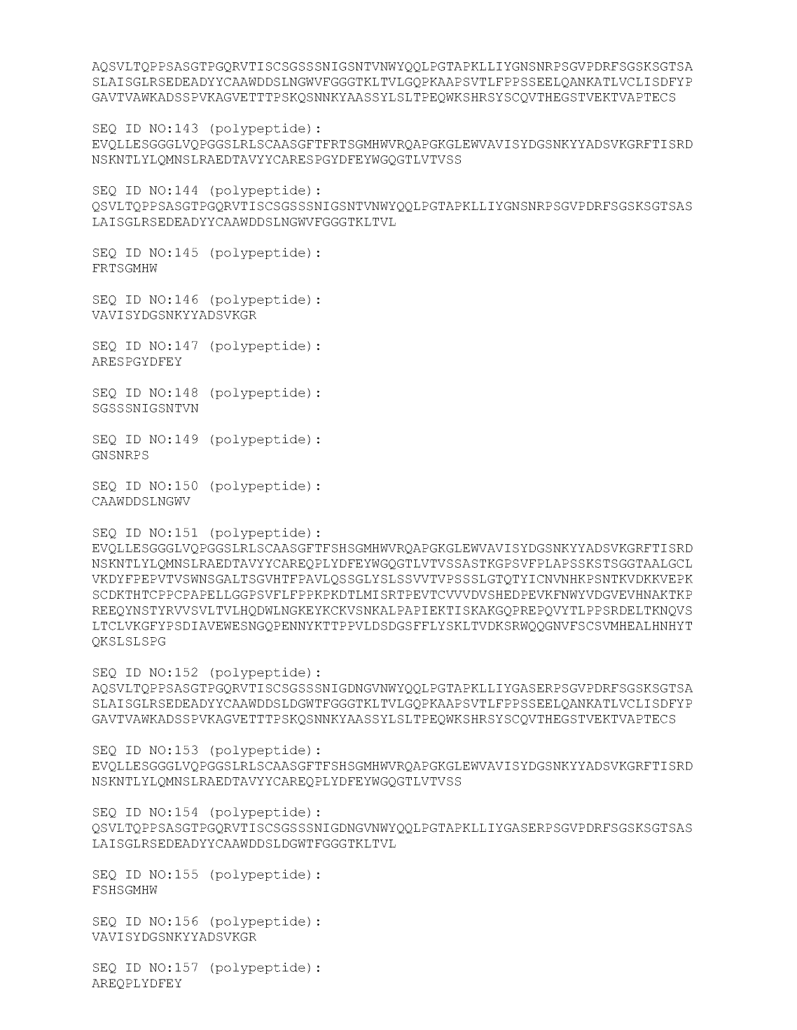

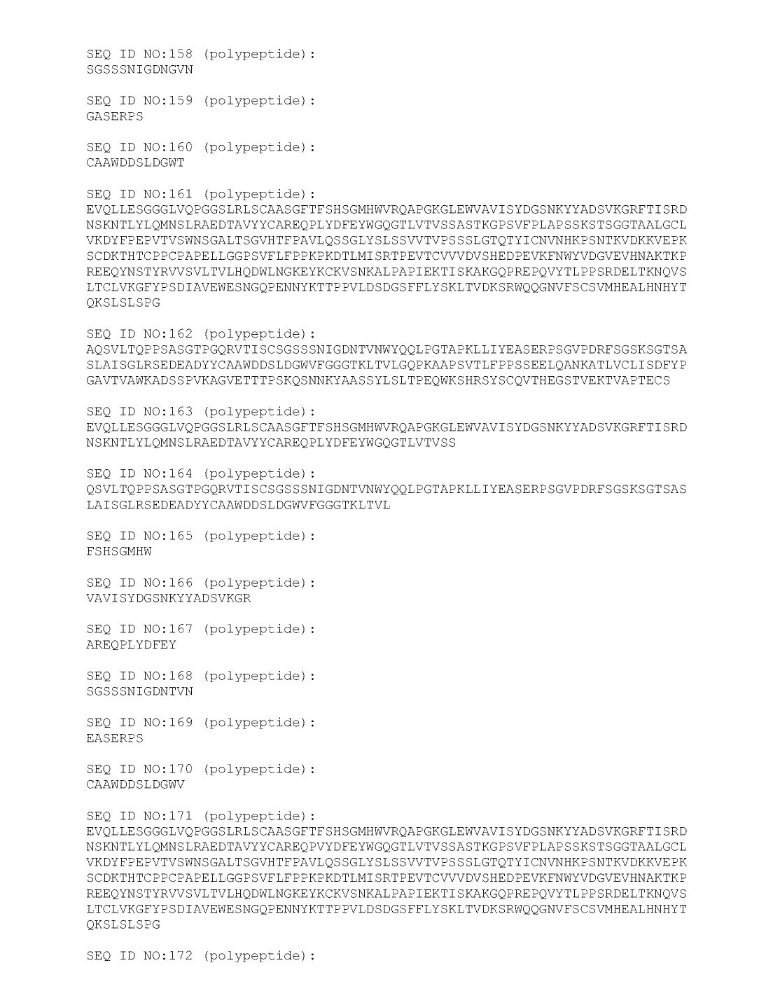

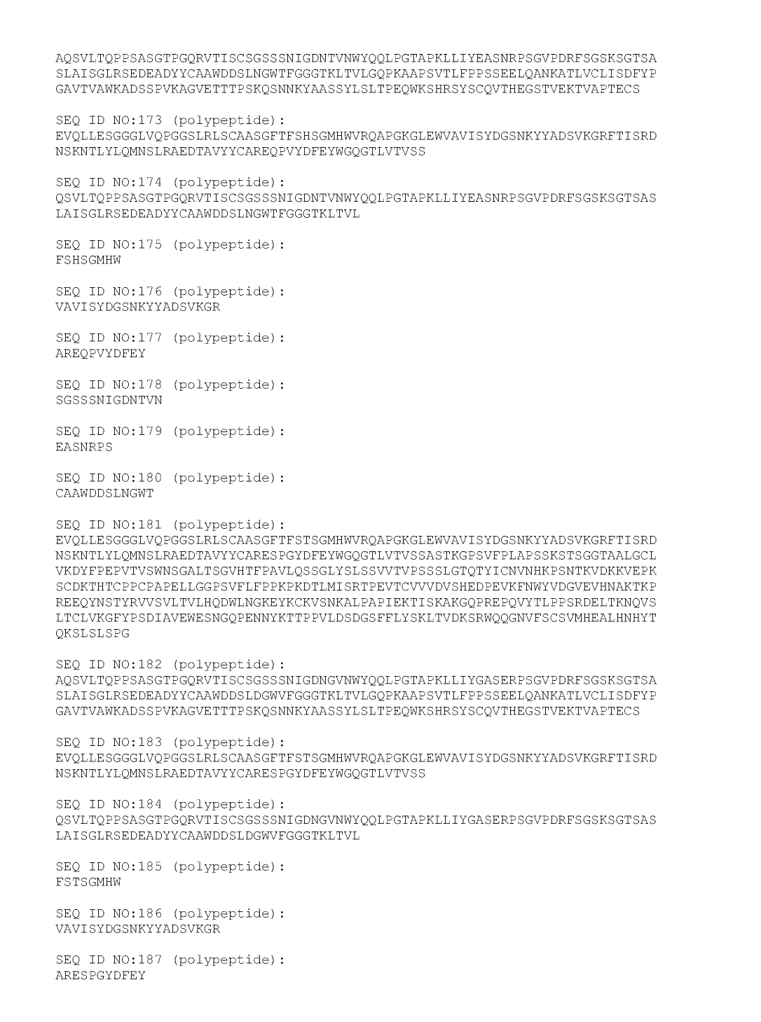

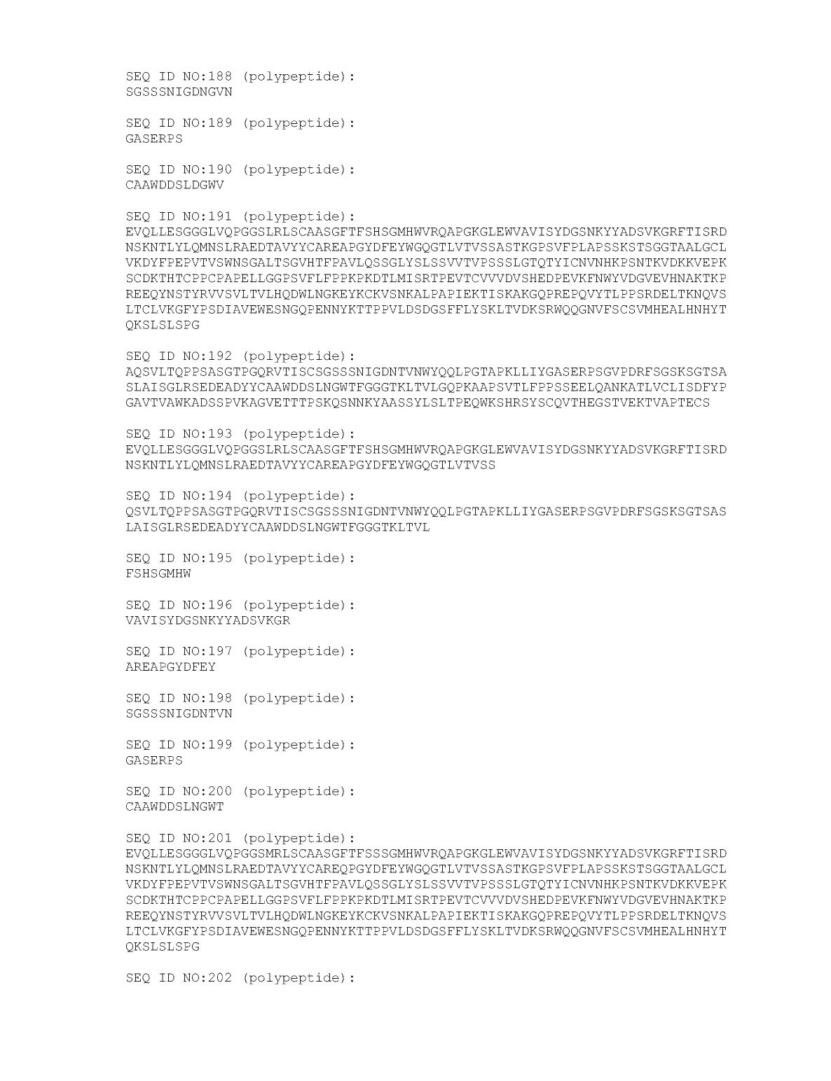

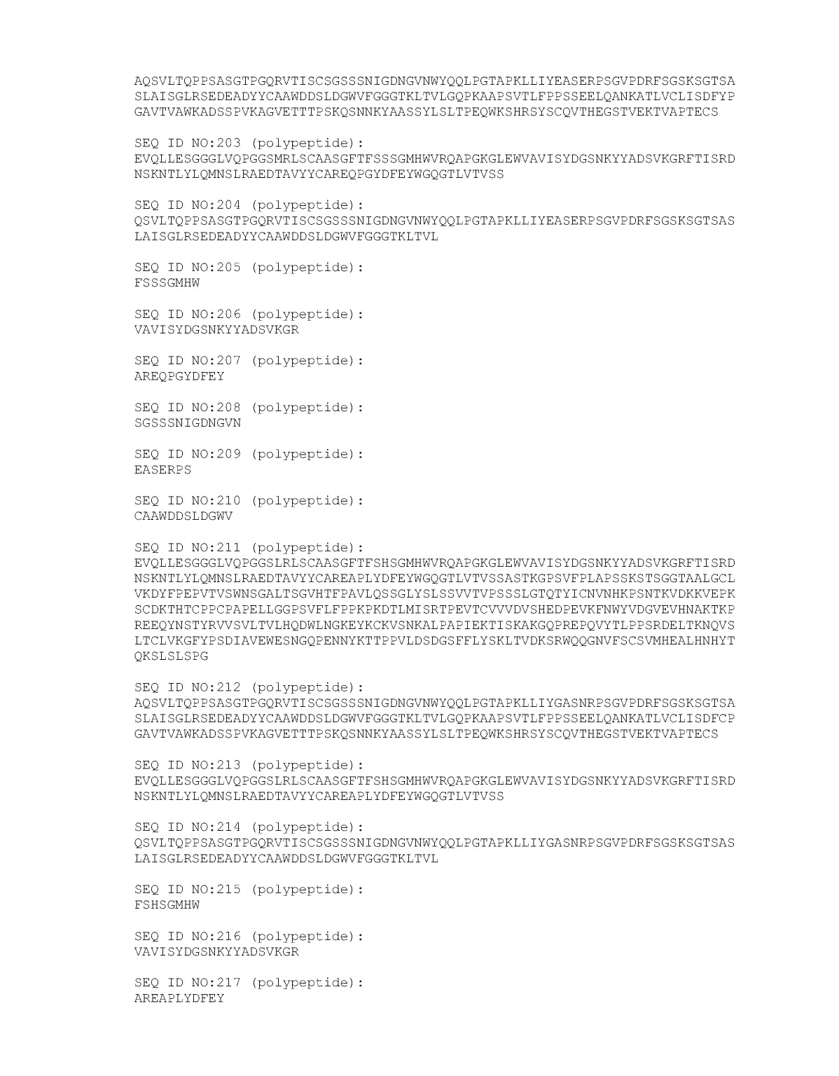

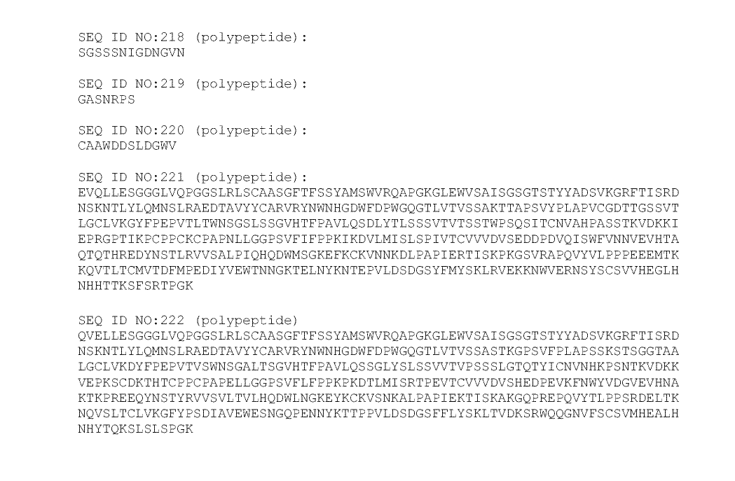

Representative results from 2 animals per group treated with anti FGFR2 antibodies are shown in FIG. 18 side by side with samples from animals treated with control IgG. Total FGFR2 as well as P-FGFR levels were strongly reduced after treatment with anti FGFR2 antibodies of this invention. Thus, the mode of action of downregulation of total FGFR2 described in the in vitro studies is also relevant in xenograft tumors after treatment with anti FGFR2 antibodies of this invention.

### Example 15

### Subcutaneous Xenograft Cancer Model with Antibody Drug Conjugates

Anti FGFR2 antibodies can be conjugated to cytotoxic small molecules using protocols that are known in the art (e.g. Liu et al., Proc Natl. Acad. Sci. (1996), 93, 8618-8623). A431 cells are maintained as adherent cultures in DMEM supplemented with 10% FBS. NOD SCID or other immunocompromised mice of 6-7 weeks age will be inoculated subcutaneously in the right flank with 1-5×10e6 cells in 0.1 ml of medium. When tumor sizes reach ca. 25 mm² antibody drug conjugates will be administered intraperitoneal 3× every 4, 7 or 10 days at a dose of 1-10 mg/kg. Control mice will be treated with PBS or an irrelevant monoclonal antibody conjugated with the same toxophore, Tumor size will be measured twice weekly with a sliding caliper. Anti-tumor efficacy will be evaluated by comparing tumor size of anti FGFR2 antibody drug conjugate treatment versus control treatment.

### Example 16

### Generation of Matured Variants of Selected Antibodies with Improved Affinities

Anti FGFR2 antibodies of this invention discovered by phage display as depicted in Table 9 were further optimized by affinity maturation.

TABLE 9
Sequences of antibodies discovered by phage display

| | SEQ ID | SEQ ID | SEQ | | | | | | | |
|---|---|---|---|---|---|---|---|---|---|---|
| | NO: | NO: | ID NO: | SEQ ID NO: | SEQ ID NO: | SEQ ID NO: | SEQ ID NO: | SEQ ID NO: | SEQ ID NO: | SEQ ID NO: |
| Antibody | HCDR1 | HCDR2 | HCDR3 | LCDR1 | LCDR2 | LCDR3 | VH Protein | VL Protein | VH Nucleotide | VL Nucleotide |
| M017-B02 | 5 | 6 | 7 | 8 | 9 | 10 | 1 | 2 | 3 | 4 |
| M021-H02 | 15 | 16 | 17 | 18 | 19 | 20 | 11 | 12 | 13 | 14 |
| M047-D08 | 25 | 26 | 27 | 28 | 29 | 30 | 21 | 22 | 23 | 24 |
| M048-D01 | 35 | 36 | 37 | 38 | 39 | 40 | 31 | 32 | 33 | 34 |
| M054-D03 | 45 | 46 | 47 | 48 | 49 | 50 | 41 | 42 | 43 | 44 |
| M054-A05 | 55 | 56 | 57 | 58 | 59 | 60 | 51 | 52 | 53 | 54 |

Antibody affinity maturation is a two-step process where saturation mutagenesis and well-based high throughput screening are combined to identify a small number of mutations resulting in affinity increases. In the first round of affinity maturation positional diversification of wild-type antibody was introduced by site-directed mutagenesis using NNK-trinucleotide cassettes (whereby N represents a 25% mix each of adenine, thymine, guanine, and cytosine nucleotides and K represents a 50% mix each of thymine and guanine nucleotides) according to BMC Biotechnology 7: 65, 2007. This way, all 20 amino acids are introduced at an individual amino acid position. This positional randomization is restricted to the six complementarity determining regions (CDRs). In the second round of affinity maturation beneficial substitutions were recombined and screened for further improvements. Examples of such variants are depicted in Table 10.

TABLE 10
Sequences of variant antibodies derived from M048-D01 and M047-D08,
respectively

| | | SEQ ID | SEQ ID | SEQ ID | SEQ ID | | | | | | |
|---|---|---|---|---|---|---|---|---|---|---|---|
| | | NO: | NO: | NO: | NO: | SEQ ID NO: | SEQ ID NO: | SEQ ID NO: | SEQ ID NO: | SEQ ID NO: | SEQ ID NO: |
| Variant of | Antibody | HCDR1 | HCDR2 | HCDR3 | LCDR1 | LCDR2 | LCDR3 | VH Protein | VL Protein | HC Protein | LC Protein |
| M048-D01 | TPP-1403 | 75 | 76 | 77 | 78 | 79 | 80 | 73 | 74 | 71 | 72 |
| | TPP-1397 | 85 | 86 | 87 | 88 | 89 | 90 | 83 | 84 | 81 | 82 |
| | TPP-1398 | 95 | 96 | 97 | 98 | 99 | 100 | 93 | 94 | 91 | 92 |
| | TPP-1399 | 105 | 106 | 107 | 108 | 109 | 110 | 103 | 104 | 101 | 102 |
| | TPP-1400 | 115 | 116 | 117 | 118 | 119 | 120 | 113 | 114 | 111 | 112 |
| | TPP-1401 | 125 | 126 | 127 | 128 | 129 | 130 | 123 | 124 | 121 | 122 |
| | TPP-1402 | 135 | 136 | 137 | 138 | 139 | 140 | 133 | 134 | 131 | 132 |
| M047-D08 | TPP-1415 | 145 | 146 | 147 | 148 | 149 | 150 | 143 | 144 | 141 | 142 |
| | TPP-1406 | 155 | 156 | 157 | 158 | 159 | 160 | 153 | 154 | 151 | 152 |
| | TPP-1407 | 165 | 166 | 167 | 168 | 169 | 170 | 163 | 164 | 161 | 162 |
| | TPP-1408 | 175 | 176 | 177 | 178 | 179 | 180 | 173 | 174 | 171 | 172 |
| | TPP-1409 | 185 | 186 | 187 | 188 | 189 | 190 | 183 | 184 | 181 | 182 |
| | TPP-1410 | 195 | 196 | 197 | 198 | 199 | 200 | 193 | 194 | 191 | 192 |
| | TPP-1411 | 205 | 206 | 207 | 208 | 209 | 210 | 203 | 204 | 201 | 202 |
| | TPP-1412 | 215 | 216 | 217 | 218 | 219 | 220 | 213 | 214 | 211 | 212 |

Two different types of ELISA were used to determine the binding improvement of mutated variants:
a) Peptide Binding ELISA: a synthetic peptide comprising the amino acid sequence of the epitope linked C-terminally to a biotinylated lysine RPSFSLVEDTTLEPEG-Ttds-Lys(Biotin) (peptide sequence derived from SEQ ID NO:63, synthesized by JPT Peptide Technology GmbH, Berlin, Germany), and
b) Recombinant Protein Binding ELISA: recombinant human FGFR2 (DNA sequence of human FGFR2 (NP 000132.3) Met 1-Glu 377, fused with a polyhistidine tag at the C-terminus; #10824-1-1081-1, Sin θ Biological Inc., Beijing, China).

Briefly, in both ELISA formats MTP plates (384 well Maxisorp, Nunc) were coated with 20 μl, anti-human IgG Fc specific (#12136; sigma) at 2.2 μg/ml for 2.5 h at 37° C. in coating buffer (#121125 Candor Bioscience GmbH). After one washing step using 50 μl PBST (phosphat buffered saline, 137 mM NaCl, 2.7 mM KCl, 10 mM Na₂FIPO₄, 2 mM KH₂PO₄, pH 7.4, 0.05% Tween20), plates were blocked with 50 μl of 10% Smart Block (#113500, Candor Bioscience GmbH) for 1 h at 20-22° C. and the washing step was repeated 3 times. Anti-FGFR2 variants were immobilized in concentrations of 0.035 μg/ml (peptide based assay) or 0.2 μg/ml (recombinant human FGFR2 protein based assay) in 10% Smart Block in PBST depending on the format and variants to be analyzed by incubation of 20 μl for one hour at 20-22° C. After one washing step using 50 μl PBST, 20 μl quadruplets of the antigen dilution series in 10% SmartBlock in PBST with a maximum concentration of 100 nM were added and incubated for 1 h at 20-22° C. and the washing step was repeated 3 times. For the detection of the biotinylated epitope peptide 20 μl of streptavidine/POD conjugate (# S5512, Sigma) in a 1:1000 dilution in 10% SmartBlock in PBST were applied for one hour at 20-22° C. For the detection of the recombinant FGFR2 protein 20 μl of anti-His/HRP conjugate (#71840, novagen) in a 1:10000 dilution in 10% SmartBlock in PBST were applied for one hour at 20-22° C. After 3 washing steps 20 μl of 10 μM amplex red substrate (# A12222, Invitrogen) in 50 mM Sodium hydrogen phosphate, pH 7.6, were added and the fluorescence signal was detected using a common fluorescence reader, e.g. Tecan M1000. EC50 values were evaluated by fitting the data (Sigmoida) dose-response, variable slope, bottom set to background; GraphPad Prism software).

Provided in Table 10 are several examples of variants with amino acid substitutions generated in the heavy and light chains of M048-D01 (TPP-1403). All variants showed strong improvement in antigen binding evaluated in two ELISA formats with different forms of antigen compared to the non CDR changed variant (Table 11).

Provided in Table 10 are several examples of variants with amino acid substitutions generated in the heavy and light chains of M047-D08 (TPP-1415). All variants showed significant improvement in antigen binding compared to the non CDR changed variant (Table 11).

The differences between both formats regarding the numeric results, EC50 and the factor of improvement, were unexpected but can be likely explained by the use of antigen in peptide or protein form and differences in the formation of the finally detected enzyme conjugate on top of the ELISA sandwich: The K_{D} of the anti-His-HRP conjugate and the His-tagged FGFR2 is not known, however it is very likely, that it is magnitudes of orders higher than the K_{D} of biotin and streptavidin (10-15 M) utilized in the detection of the epitope peptide. Consequently the sensitivity for the bound peptide is significant higher than the sensitivity for the His-tagged protein leading to the potential of determining smaller EC50. In addition or alternatively the differences may be caused by deviations in the interaction of the anti-FGFR2 antibody with both antigens despite their identical sequence over a stretch of 15 amino acids; firstly the chemistry of the C-terminal following part of the molecules is very different, secondly the 15 amino acids might take a 3D conformation not identical to the corresponding region in the FGFR2 protein. Both explanations could refer to the differences between the peptide and protein based ELISA showing smaller EC50 values in the peptide ELISA format.

The data sets in Table 11 clearly indicate that M048-D01 (TPP-1403) binds FGFR2 at its N-terminal sequence as represented in the epitope peptide, and that several variants with amino acid substitutions in the CDRs surprisingly do the same even with higher affinity. Notably, the substitution N102I is present in five of the six other variants of TPP-1403 accompanied by several other substitutions in CDR-L1, -L2, -L3, -1-12 and/or -1-13, but not in TPP-1399 showing surprisingly a lysine (K) at position HC-102.

The data sets in Table 11 indicate that M047-D08 (TPP-1415) binds FGFR2 at its N-terminal sequence as represented in the epitope peptide, and that several variants with amino acid substitutions in the CDRs surprisingly do the same even with higher affinity. Variants of M047-D08 (TPP-1415) with multiple amino acid substitutions showed approximately four- to forty-fold improved binding, TPP-1409 least (2.1 nM) and TPP-1406 (0.22 nM) most. Notably three of them have a G102L (TPP-1406, -1407 and -1412) and one a G102V (TPP-1408) substitution accompanied by several other substitutions in CDR-L1, -L2, -L3, -H1 and/or -H3.

TABLE 11
Peptide binding ELISA results, Protein binding ELISA results and internalization
efficacy data of variant antibodies derived from M048-D01 and M047-D08,
respectively

| | | | | | | | Internalization |
|---|---|---|---|---|---|---|---|
| | | Peptide Binding | | | Protein Binding | | efficacy |
| | | EC50 | fold EC50 | fold | EC50 | fold EC50 | fold |
| Variant of | | [nM] | reduction | signal | [nM] | reduction | improvement |
| M048-D01 | TPP-1397 | 0.004 | >2300 | | 0.20 | 40 | 1.9 |
| | TPP-1398 | 0.005 | >2000 | | 0.20 | 39 | 1.2 |
| | TPP-1399 | 0.006 | >1500 | | 0.21 | 39 | 2.0 |
| | TPP-1400 | 0.009 | >1100 | | 0.48 | 17 | 1.9 |
| | TPP-1401 | 0.006 | >1700 | | 0.20 | 39 | 2.1 |
| | TPP-1402 | 0.007 | >1500 | | 0.22 | 37 | 2.4 |
| | TPP-1403 | >10 | 1 | | 8.0 | 1 | 1 |
| M047-D08 | TPP-1406 | | | | 0.22 | 37 | 0.9 |
| | TPP-1407 | | | | 0.23 | 35 | 1.1 |
| | TPP-1408 | 2.5 | >4 | 10 | 0.29 | 27 | 1 |
| | TPP-1409 | | | | 2.1 | 3.8 | 1 |
| | TPP-1410 | | | | 0.66 | 12 | 0.9 |
| | TPP-1411 | | | | 0.61 | 13 | 0.7 |
| | TPP-1412 | | | | 0.29 | 28 | 1.5 |
| | TPP-1415 | >10 | 1 | 1 | 8.0 | 1 | 1 |

In addition in Table 11 the improvements of internalization efficacy of maturated anti FGFR2 antibodies are summarized. The improvement factor is calculated based on comparison of total granule intensity/cell achieved by internalization and degradation of maturated antibodies to the corresponding value of the parental antibody. Equal findings are achieved by comparison of granule count/cell resulting in the identical ranking of antibodies. Experimental details are described in Example 12. Notably all matured variants of M048-D01 (TPP-1403) showed an improved internalization efficacy (1.9 to 2.4 fold). In case of M047-D08 (TPP-1415) variant TPP-1412 showed a 1.5 fold improved internalization efficacy. Internalization is an important feature of the antibodies of this invention.

With the variants provided for M047-D08 and M048-D01 it could clearly be demonstrated that variants of these antibodies can have similar or improved properties if the epitope is maintained.

### Example 17

### Determination of Competition with Other Anti-FGFR2 Antibodies

To analyze the competition between anti-FGFR2 antibodies according to the invention and anti-FGFR2 antibodies described in the art, different antibodies were evaluated in a competitive ELISA format:

MTP plates (384 well Maxisorp, Nunc) were coated with 20 μl of 2 μg/ml anti-human IgG (Fc specific (#12136; sigma) in coating buffer (#121125 Candor Bioscience GmbH) at 4° C. over night. After one washing step using 50 μl PBST (phosphat buffered saline, 137 mM NaCl, 2.7 mM KCl, 10 mM Na2HPO4, 2 mM KH2PO4, pH 7.4, 0.05% Tween20), plates were blocked with 50 μl of 100% Smart Block (#113500, Candor Bioscience GmbH) for 1 h at 20-22° C. and the washing step was repeated 3 times. M048-D01-hIgG1 was immobilized in a concentrations of 1 μg/ml in 10% Smart Block in PBST by incubation of 20 μl for one hour at 20-22° C. (indicated in Table 12; row 2 with M048-D01-hIgG1 capture yes); control wells without M048-D01-hIgG1 were incubated with 10% Smart Block in PBST only (indicated in Table 12; row 2 with M048-D01-hIgG1 capture no). The immobilization step was followed by three washing steps using 50 μl PBST. 20 μl quadruplets of the pre-incubated (1 h at 20-22° C.) antigen/antibody mix composed by recombinant human FGFR2, 10 nM (#10824-H108H, SinoBiological) and anti-FGFR2 IgG in a 5-fold dilution series (1000 to 0.064 nM) in 10% SmartBlock in PBST were added and incubated for 1 h at 20-22° C., followed by three washing steps.

For the detection of the recombinant human FGFR220 μl of anti-His/HRP conjugate (#71840, novagen) in a 1:10000 dilution in 10% SmartBlock in PBST were applied for one hour at 20-22° C. After 3 washing steps 20 μl of 10 μM amplex red substrate (#A12222, Invitrogen) in 50 mM Sodium hydrogen phosphate, pH 7.6, were added and the fluorescence signal was detected using a common fluorescence reader, e.g. Tecan M1000.

Three different FGFR2 binding antibodies called GAL-FR21, GAL-FR22 and GAL-FR23 (described in WO2010/054265 and Zhao et al. (Clin Cancer Res. 2010, 16:5750-5758)) have been described to bind to different domain epitopes. For evaluation of difference with these antibodies competition assays were performed.

Due to the different isotypes of the analyzed antibodies the competition ELISA format has to ensure an equally and directly comparable detection of the competition situation without superposition of additional effects due to use of different detection antibodies or different affinities of a single detection antibody to the different IgG-isotypes. The ELISA format described above fulfills this criterion by detection of the FGFR2 antigen via its His-tag instead of the detection of bound mouse or human IgG1 or IgG2a. The immobilization of M048-D01-hIgG1 is specific with respect to its human Fc portion, otherwise significant amounts of FGFR2 would have been detected in ELISA plate wells coated with anti-human IgG (Fc specific), but not supplied with M048-D01-hIgG1a potential binding of mouse anti-FGFR2-IgG to anti-human IgG (Fc specific) and subsequent binding of FGFR2 was not detected (Table 12, columns 8-11). Additionally, no significant unspecific binding of FGFR2 to the immobilized anti-human IgG (Fc specific) was observed (column 2). The “self-competition” of M048-D01-hIgG1 worked very clearly (column 6), and the same is true for M048-D01-mIgG2a, (column 7). The observation, that neither GAL-FR21, -FR22 nor -FR23 showed dose dependent reduction in detectable FGFR2 (column 3-5) as M048-D01-hIgG1 and M048-D01-mIgG2a did with an >50% decreased signal at 1.25 and 0.63 nM and higher concentrations of competing antibody, respectively, demonstrates the differences between M048-D01 and the three GAL antibodies. In contrast, after pre-incubation the monomeric FGFR2 (10 nM) with GAL-FR22 and GAL-FR23, but not with GAL-FR21, the amount of detectable FGFR2 appeared to be significantly increased. Since the GAL antibodies are neither fused to a His-Tag, checked by Western analysis, nor captured by the anti-human IgG (Fc specific), the most likely explanation is, that monomeric FGFR2 can be dimerized by the pre-incubation with antibodies leading to an avidity effect in the subsequent binding of FGFR2 to the immobilized M048-D01-hIgG1. The situation of immobilized M048-D01-hIgG1 bound directly to FGFR2 and mediated by this indirectly to the dimerizing antibodies GAL-FR22 and GAL-FR23 would further illustrate, that M048-D01-hIgG1 binds to a complete different FGFR2 epitope than the GAL antibodies, otherwise a simultaneous binding event could not occur. Notably, GAL-FR21 did not increase the amount of detectable FGFR2. This difference can be plausibly interpreted by taking the more particular description of the GAL antibodies as described in WO2010/054265 into account: Gal-FR22 binds to an epitope in D2-D3IIIa, and GAL-FR23 binds to one all or partly located in D1; both regions represented in the used recombinant human FGFR2-IIIc molecule. But for GAL-FR21 the epitope is described to be located in D3-IIIb, a sequence stretch not represented in this FGFR2-IIIc isoform; consequently GAL-FR21 is not able to bind the antigen and mediate an avidity effect. As shown, in none of the assays competition between M048-D01-hIgG1 and one of the GAL antibodies was observed.

TABLE 12
Antibody Competition ELISA. The average signals are given in relative to the
corresponding value for 10 nM FGFR2 determined in the calibration series (column 1)

| | column | | | | | | | | | | | |
|---|---|---|---|---|---|---|---|---|---|---|---|---|
| | 1 | 2 | | 3 | 4 | 5 | 6 | 7 | 8 | 9 | 10 | 11 |
| | M048-D01-hIgG1capture | | | | | | | | | | | |
| | | | | | | | yes | yes | no | | | |
| nM | | | nM | yes | yes | yes | M048-D01- | M048-D01- | M048-D01- | no | no | no |
| FGFR2 | yes | no | competitor | Gal FR-21 | Gal FR-22 | Gal FR-23 | hIgG1 | mIgG2a | mIgG2a | Gal FR-21 | Gal FR-22 | Gal FR-23 |
| 20 | 148% | 3% | 1000 | 77% | 198% | 219% | 3% | 3% | 3% | 3% | 3% | 5% |
| 10 | 100% | 2% | 200 | 77% | 250% | 265% | 4% | 3% | 2% | 2% | 2% | 3% |
| 5 | 51% | 2% | 40 | 77% | 284% | 297% | 20% | 5% | 1% | 2% | 2% | 3% |
| 2.5 | 23% | 2% | 8 | 87% | 287% | 294% | 30% | 10% | 2% | 2% | 2% | 3% |
| 1.25 | 12% | 2% | 1.6 | 91% | 248% | 222% | 44% | 18% | 2% | 2% | 2% | 3% |
| 0.63 | 6% | 2% | 0.32 | 81% | 167% | 151% | 63% | 34% | 1% | 2% | 1% | 3% |
| 0.31 | 18% | 2% | 0.06 | 76% | 92% | 106% | 81% | 60% | 2% | 2% | 2% | 3% |
| 0.16 | 4% | 3% | 0 | 79% | 84% | 93% | 93% | 82% | 3% | 3% | 3% | 5% |

The results of competition experiments, as described above, are supported by the observation, that all three GAL antibodies including GAL-FR23 (epitope all or partly located in D1) show no binding to the synthetic peptide of the extracellular N-terminal epitope of FGFR2 (SEQ ID NO:63) comprising the amino acid sequence of the epitope C-terminally linked to a biotinylated lysine (¹RPSFSLVEDTTLEPE¹⁵G-Ttds-Lys(Biotin)) even in the highest concentration in the IgG titration series applied (600 nM), whereas the strong binding of M048-D01-hIgG1 (detected by anti-human IgG (Fc specific) POD conjugate; # A5175, sigma) and M048-D01-mIgG2a, resulted in EC50 in the range ≦1 nM (detailed data not shown). For the detection of mouse antibodies anti-mouse IgG (Fc specific) POD conjugate (#715-35-15, jakson) was used, checked positively for its ability to detect GAL-FR21, -FR22, -FR23 and M048-D01-mIgG2a bound to FGFR2-IIIb alpha.

1. An isolated antibody or antigen-binding fragment thereof which reduces the cell surface expression of FGFR2 after binding to FGFR2 in cell lines SNU16 (ATCC-CRL-5974) and MFM223 (ECACC-98050130) which overexpress FGFR2 and in cell lines AN3-CA (DSMZ-ACC 267) and MFE-296 (ECACC-98031101) which express mutated FGFR2.

2. An isolated antibody or antigen-binding fragment thereof specifically binding to the extracellular N-terminal epitope (¹RPSFSLVEDTTLEPE¹⁵) of FGFR2 as presented by SEQ ID NO:63.

3. An isolated antibody or antigen-binding fragment thereof according to claim 2 wherein binding of the antibody to the extracellular N-terminal epitope (SEQ ID NO:63) is mediated by at least one epitope residue selected from the group of residues consisting of Arg 1, Pro 2, Phe 4, Ser 5, Leu 6, and Glu 8.

4. An isolated antibody or antigen-binding fragment thereof according to any one of claims 2-3 wherein the antibody or antigen-binding fragment thereof loses more than 50% of its ELISA signal by changing of at least one of the amino acid residues in the N-terminal epitope (¹RPSFSLVEDTTLEPE¹⁵) of FGFR2 into an Alanine
a. said residue selected from the group Pro 2, Leu 6 and Glu 8, or
b. said residue selected from the group Arg 1, Pro 2, Phe 4 and Ser 5.

5. The antibody or antigen-binding fragment according to any one of claims 1 to 4, wherein the antibody or antigen-binding fragment competes in binding to FGFR2 with at least one antibody selected from the group “M048-D01”, “M047-D08”, “M017-B02”, “M021-H02”, “M054-A05”, “M054-D03”, “TPP-1397”, “TPP-1398”, “TPP-1399”, “TPP-1400”, “TPP-1401”, “TPP-1402”, “TPP-1403”, “TPP-1406”, “TPP-1407”, “TPP-1408”, “TPP-1409”, “TPP-1410”, “TPP-1411”, “TPP-1412”, and “TPP-1415”.

6. The antibody or antigen-binding fragment according to according to any one of claims 5, wherein the amino acid sequence of the antibody or antigen-binding fragment is at least 50%, 55%, 60% 70%, 80%, 90, or 95% identical to at least one CDR sequence of “M048-D01”, “M047-D08”, “M017-B02”, “M0214102”, “M054-A05”, “M054-D03”, “TPP-1397”, “TPP-1398”, “TPP-1399”, “TPP-1400”, “TPP-1401”, “TPP-1402”, “TPP-1403”, “TPP-1406”, “TPP-1407”, “TPP-1408”, “TPP-1409”, “TPP-1410”, “TPP-1411”, “TPP-1412”, or “TPP-1415”, or at least 50%, 60%, 70%, 80%, 90%, 92% or 95% identical to the VH or VL sequence of “M048-D01”, “M047-D08”, “M017-B02”, “M021-H02”, “M054-A05”, “M054-D03”, “TPP-1397”, “TPP-1398”, “TPP-1399”, “TPP-1400”, “TPP-1401”, “TPP-1402”, “TPP-1403”, “TPP-1406”, “TPP-1407”, “TPP-1408”, “TPP-1409”, “TPP-1410”, “TPP-1411”, “TPP-1412”, or “TPP-1415”.

7. The antibody or antigen-binding fragment according to any one of claims 5-6, wherein the antibody or antigen-binding fragment comprises at least one CDR sequence or at least one variable heavy chain or light chain sequence as depicted in Table 9 and Table 10.

8. The antibody or antigen-binding fragment according to claim 1 to 7 comprising
a. the variable heavy chain CDR sequences as presented by SEQ ID NO: 5-7 and the variable light chain CDR sequences presented by SEQ ID NO: 8-10, or
b. the variable heavy chain CDR sequences as presented by SEQ ID NO: 15-17 and the variable light chain CDR sequences presented by SEQ ID NO: 18-20, or
c. the variable heavy chain CDR sequences as presented by SEQ ID NO: 25-27 and the variable light chain CDR sequences presented by SEQ ID NO: 28-30, or
d. the variable heavy chain CDR sequences as presented by SEQ ID NO: 35-37 and the variable light chain CDR sequences presented by SEQ ID NO: 38-40, or
e. the variable heavy chain CDR sequences as presented by SEQ ID NO: 45-47 and the variable light chain CDR sequences presented by SEQ ID NO: 48-50, or
f. the variable heavy chain CDR sequences as presented by SEQ ID NO: 55-57 and the variable light chain CDR sequences presented by SEQ ID NO: 58-60, or
g. the variable heavy chain CDR sequences as presented by SEQ ID NO: 75-77 and the variable light chain CDR sequences presented by SEQ ID NO: 78-80, or
h. the variable heavy chain CDR sequences as presented by SEQ ID NO: 85-87 and the variable light chain CDR sequences presented by SEQ ID NO: 88-90, or
i. the variable heavy chain CDR sequences as presented by SEQ ID NO: 95-97 and the variable light chain CDR sequences presented by SEQ ID NO: 98-100, or
j. the variable heavy chain CDR sequences as presented by SEQ ID NO: 105-107 and the variable light chain CDR sequences presented by SEQ ID NO: 108-110, or
k. the variable heavy chain CDR sequences as presented by SEQ ID NO: 115-117 and the variable light chain CDR sequences presented by SEQ ID NO: 118-120, or
l. the variable heavy chain CDR sequences as presented by SEQ ID NO: 125-127 and the variable light chain CDR sequences presented by SEQ ID NO: 128-130, or
m. the variable heavy chain CDR sequences as presented by SEQ ID NO: 135-137 and the variable light chain CDR sequences presented by SEQ ID NO: 138-140, or
n. the variable heavy chain CDR sequences as presented by SEQ ID NO: 145-147 and the variable light chain CDR sequences presented by SEQ ID NO: 148-150, or
o. the variable heavy chain CDR sequences as presented by SEQ ID NO: 155-157 and the variable light chain CDR sequences presented by SEQ ID NO: 158-160, or
p. the variable heavy chain CDR sequences as presented by SEQ ID NO: 165-167 and the variable light chain CDR sequences presented by SEQ ID NO: 168-170, or
q. the variable heavy chain CDR sequences as presented by SEQ ID NO: 175-177 and the variable light chain CDR sequences presented by SEQ ID NO: 178-180, or
r. the variable heavy chain CDR sequences as presented by SEQ ID NO: 185-187 and the variable light chain CDR sequences presented by SEQ ID NO: 188-190, or
s. the variable heavy chain CDR sequences as presented by SEQ ID NO: 195-197 and the variable light chain CDR sequences presented by SEQ ID NO: 198-200, or
t. the variable heavy chain CDR sequences as presented by SEQ ID NO: 205-207 and the variable light chain CDR sequences presented by SEQ ID NO: 208-210, or
u. the variable heavy chain CDR sequences as presented by SEQ ID NO: 215-217 and the variable light chain CDR sequences presented by SEQ ID NO: 218-220.

9. The antibody or antigen-binding fragment according to claims 1-8 comprising
a. a variable heavy chain sequence as presented by SEQ ID NO:1 and a variable light chain sequences as presented by SEQ ID NO:2, or
b. a variable heavy chain sequence as presented by SEQ ID NO:11 and a variable light chain sequences as presented by SEQ ID NO:12, or
c. a variable heavy chain sequence as presented by SEQ ID NO:21 and a variable light chain sequences as presented by SEQ ID NO:22, or
d. a variable heavy chain sequence as presented by SEQ ID NO:31 and a variable light chain sequences as presented by SEQ ID NO:32, or
e. a variable heavy chain sequence as presented by SEQ ID NO:41 and a variable light chain sequences as presented by SEQ ID NO:42, or
f. a variable heavy chain sequence as presented by SEQ ID NO:51 and a variable light chain sequences as presented by SEQ ID NO:52, or
g. a variable heavy chain sequence as presented by SEQ ID NO:73 and a variable light chain sequences as presented by SEQ ID NO:74, or
h. a variable heavy chain sequence as presented by SEQ ID NO:83 and a variable light chain sequences as presented by SEQ ID NO:84, or
i. a variable heavy chain sequence as presented by SEQ ID NO:93 and a variable light chain sequences as presented by SEQ ID NO:94, or
j. a variable heavy chain sequence as presented by SEQ ID NO:103 and a variable light chain sequences as presented by SEQ ID NO:104, or
k. a variable heavy chain sequence as presented by SEQ ID NO:113 and a variable light chain sequences as presented by SEQ ID NO:114, or
l. a variable heavy chain sequence as presented by SEQ ID NO:123 and a variable light chain sequences as presented by SEQ ID NO:124, or
m. a variable heavy chain sequence as presented by SEQ ID NO:133 and a variable light chain sequences as presented by SEQ ID NO:134, or
n. a variable heavy chain sequence as presented by SEQ ID NO:143 and a variable light chain sequences as presented by SEQ ID NO:144, or
o. a variable heavy chain sequence as presented by SEQ ID NO:153 and a variable light chain sequences as presented by SEQ ID NO:154, or
p. a variable heavy chain sequence as presented by SEQ ID NO:163 and a variable light chain sequences as presented by SEQ ID NO:164, or
q. a variable heavy chain sequence as presented by SEQ ID NO:173 and a variable light chain sequences as presented by SEQ ID NO:174, or
r. a variable heavy chain sequence as presented by SEQ ID NO:183 and a variable light chain sequences as presented by SEQ ID NO:184, or
s. a variable heavy chain sequence as presented by SEQ ID NO:193 and a variable light chain sequences as presented by SEQ ID NO:194, or
t. a variable heavy chain sequence as presented by SEQ ID NO:203 and a variable light chain sequences as presented by SEQ ID NO:204, or
u. a variable heavy chain sequence as presented by SEQ ID NO:213 and a variable light chain sequences as presented by SEQ ID NO:214.

10. The antibody according to any one of the preceding claims, which is an IgG antibody.

11. The antigen-binding fragment according to any one of the preceding claims, which is an scFv, Fab, Fab′ fragment or a F(ab′)₂ fragment.

12. The antibody or antigen-binding fragment according to any one of the preceding claims, which is a monoclonal antibody or antigen-binding fragment.

13. The antibody or antigen-binding fragment according to any one of the preceding claims, which is human, humanized or chimeric antibody or antigen-binding fragment.

14. An antibody-drug conjugate, comprising an antibody or antigen binding fragment thereof according to claims 1 to 13.

15. An isolated nucleic acid sequence that encodes the antibody or antigen-binding fragment according to claims 1 to 13.

16. A vector comprising a nucleic acid sequence according to claim 15.

17. An isolated cell expressing an antibody or antigen-binding fragment according to any one of the claims 1 to 13 and/or comprising a nucleic acid according to claim 15 or a vector according to claim 16.

18. An isolated cell according to claim 17, wherein said cell is a prokaryotic or an eukaryotic cell.

19. A method of producing an antibody or antigen-binding fragment according to any one of the claims 1-13 comprising culturing of a cell according to claim 18 and purification of the antibody or antigen-binding fragment.

20. An antibody or antigen-binding fragment according to claims 1-13 or an antibody-drug conjugate according to claim 14 as a medicament.

21. An antibody or antigen antigen-binding fragment according to claims 1-13 as a diagnostic agent.

22. An antibody or antigen-binding fragment according to claims 1-13 or an antibody-drug conjugate according to claim 14 as a medicament for the treatment of cancer.

23. A pharmaceutical composition comprising an antibody or antigen-binding fragment according to claims 1-13 or an antibody-drug conjugate according to claim 14.

24. A combination of a pharmaceutical composition according to claim 23 and one or more therapeutically active compounds.

25. A method for treating a disorder or condition associated with the undesired presence of FGFR2, comprising administering to a subject in need thereof an effective amount of the pharmaceutical composition according to claim 23 or a combination according to claim 24.


SEQUENCE LISTING (222 sequences)

SEQ ID NO: 1 - protein, 118 aa, Homo Sapiens
EVQLLESGGGLVQPGGSLRLSCAASGFTFRTSGMHWVRQAPGKGLEWVAVISYDGSNKYYADSVKGRFTISRDNSKNTLYLQMNSLRAEDTAVYYCARESPGYDFEYWGQGTLVTVSS

SEQ ID NO: 2 - protein, 110 aa, Homo Sapiens
QSVLTQPPSASGTPGQRVTISCSGSSSNIGSNTVNWYQQLPGTAPKLLIYDNNKRPSGVPDRFSGSKSGTSASLAISGLRSEDEADYYCAAWDDSLSGWVFGGGTKLTVL

SEQ ID NO: 3 - DNA, 354 bp, Homo Sapiens
gaggtgcagctgttggagtctgggggaggcttggtacagcctggggggtccctgagactctcctgtgcagcctctggattcaccttccgtacctctggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagccgaggacactgccgtgtattactgtgcgagagaatcccccggctacgattttgagtattggggccagggtaccctggtcaccgtgagctca

SEQ ID NO: 4 - DNA, 330 bp, Homo Sapiens
cagtctgtgctgactcagccaccctcagcgtctgggacccccgggcagagggtcaccatctcttgttctggaagcagctccaacatcggaagtaatactgtaaactggtatcagcagctcccaggaacggcccccaaactcctcatctatgacaataataagcgaccctcaggggtccctgaccgattctctggctccaagtctggcacctcagcctccctggccatcagtgggctccggtccgaggatgaggctgattattactgtgcagcatgggatgacagcctgagtggttgggtgttcggcggaggaaccaagctgacggtccta

SEQ ID NO: 5 - protein, 8 aa, Homo Sapiens
FRTSGMHW

SEQ ID NO: 6 - protein, 20 aa, Homo Sapiens
VAVISYDGSNKYYADSVKGR

SEQ ID NO: 7 - protein, 11 aa, Homo Sapiens
ARESPGYDFEY

SEQ ID NO: 8 - protein, 13 aa, Homo Sapiens
SGSSSNIGSNTVN

SEQ ID NO: 9 - protein, 7 aa, Homo Sapiens
DNNKRPS

SEQ ID NO: 10 - protein, 12 aa, Homo Sapiens
CAAWDDSLSGWV

SEQ ID NO: 11 - protein, 117 aa, Homo Sapiens
EVQLLESGGGLVQPGGSLRLSCAASGFTFSNYGIHWVRQAPGKGLEWVAVISYDGSNKYYADSVKGRFTISRDNSKNTLYLQMNSLRAEDTAMYYCARDSSSAEDYWGQGTLVTVSS

SEQ ID NO: 12 - protein, 110 aa, Homo Sapiens
QSVLTQPPSASGTPGQRVTISCSGSSSNTGSNTVNWYQQLPGTAPKLLIYRNNQRPSGVPDRFSGSKSGTSASLAISGLRSEDEADYYCETWDSNTRGVVFGGGTKLTVL

SEQ ID NO: 13 - DNA, 351 bp, Homo Sapiens
gaggtgcagctgttggagtctgggggaggcttggtacagcctggggggtccctgagactctcctgtgcagcctctggattcaccttcagtaactatggcatacactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagccgaggacactgccatgtattactgtgcgcgagacagcagttcggcggaggactactggggccaaggtaccctggtcaccgtgagctca

SEQ ID NO: 14 - DNA, 330 bp, Homo Sapiens
cagtctgtgctgactcagccaccctcagcgtctgggacccccgggcagagggtcaccatctcttgttctggaagcagctccaacaccggaagcaatactgtaaactggtatcagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgagacctgggacagtaacactcggggggtggtattcggcggaggaaccaagctgacggtccta

SEQ ID NO: 15 - protein, 8 aa, Homo Sapiens
FSNYGIHW

SEQ ID NO: 16 - protein, 20 aa, Homo Sapiens
VAVISYDGSNKYYADSVKGR

SEQ ID NO: 17 - protein, 10 aa, Homo Sapiens
ARDSSSAEDY

SEQ ID NO: 18 - protein, 13 aa, Homo Sapiens
SGSSSNTGSNTVN

SEQ ID NO: 19 - protein, 7 aa, Homo Sapiens
RNNQRPS

SEQ ID NO: 20 - protein, 12 aa, Homo Sapiens
CETWDSNTRGVV

SEQ ID NO: 21 - protein, 118 aa, Homo Sapiens; identical to SEQ ID NO: 1

SEQ ID NO: 22 - protein, 110 aa, Homo Sapiens
QSVLTQPPSASGTPGQRVTISCSGSSSNIGSNTVNWYQQLPGTAPKLLIYGNSNRPSGVPDRFSGSKSGTSASLAISGLRSEDEADYYCAAWDDSLNGWVFGGGTKLTVL

SEQ ID NO: 23 - DNA, 354 bp, Homo Sapiens; identical to SEQ ID NO: 3

SEQ ID NO: 24 - DNA, 330 bp, Homo Sapiens
cagtctgtgctgactcagccaccctcagcgtctgggacccccgggcagagggtcaccatctcttgttctggaagcagctccaacatcggaagtaatactgtaaactggtatcagcagctcccaggaacggcccccaaactcctcatctatggtaacagcaatcggccctcaggggtccctgaccgattctctggctccaagtctggcacctcagcctccctggccatcagtgggctccggtccgaggatgaggctgattattactgtgcagcatgggatgacagcctgaatggttgggtgttcggcggaggaaccaagctgacggtccta

SEQ ID NO: 25 - protein, 8 aa, Homo Sapiens
FRTSGMHW

SEQ ID NO: 26 - protein, 20 aa, Homo Sapiens
VAVISYDGSNKYYADSVKGR

SEQ ID NO: 27 - protein, 11 aa, Homo Sapiens
ARESPGYDFEY

SEQ ID NO: 28 - protein, 13 aa, Homo Sapiens
SGSSSNIGSNTVN

SEQ ID NO: 29 - protein, 7 aa, Homo Sapiens
GNSNRPS

SEQ ID NO: 30 - protein, 12 aa, Homo Sapiens
CAAWDDSLNGWV

SEQ ID NO: 31 - protein, 122 aa, Homo Sapiens
EVQLLESGGGLVQPGGSLRLSCAASGFTFSSYAMSWVRQAPGKGLEWVSAISGSGTSTYYADSVKGRFTISRDNSKNTLYLQMNSLRAEDTAVYYCARVRYNWNHGDWFDPWGQGTLVTVSS

SEQ ID NO: 32 - protein, 110 aa, Homo Sapiens
QSVLTQPPSASGTPGQRVTISCSGSSSNIGNNYVSWYQQLPGTAPKLLIYENYNRPAGVPDRFSGSKSGTSASLAISGLRSEDEADYYCSSWDDSLNYWVFGGGTKLTVL

SEQ ID NO: 33 - DNA, 366 bp, Homo Sapiens
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtactagcacatactacgcagactccgtgaagggccggttcaccatctccagagacaattccaagaacacgctgtatctgcaaatgaacagcctgagagccgaggacactgccgtgtattactgtgcgagagtccgttataactggaaccacggagactggttcgacccctggggccaaggtaccctggtcaccgtgagctca

SEQ ID NO: 34 - DNA, 330 bp, Homo Sapiens
cagtctgtgctgactcagccaccctcagcgtctgggacccccgggcagagggtcaccatctcctgctctggaagcagctccaacattgggaataattatgtatcctggtatcagcagctcccaggaacggcccccaaactcctcatctatgagaattacaaccgacccgcaggggtccctgaccgattctctggctccaagtctggcacctcagcctccctggccatcagtgggctccggtccgaggatgaggctgattattactgttcatcgtgggatgacagcctgaattattgggtgttcggcggaggaaccaagctgacggtccta

SEQ ID NO: 35 - protein, 8 aa, Homo Sapiens
FSSYAMSW

SEQ ID NO: 36 - protein, 20 aa, Homo Sapiens
VSAISGSGTSTYYADSVKGR

SEQ ID NO: 37 - protein, 15 aa, Homo Sapiens
ARVRYNWNHGDWFDP

SEQ ID NO: 38 - protein, 13 aa, Homo Sapiens
SGSSSNIGNNYVS

SEQ ID NO: 39 - protein, 7 aa, Homo Sapiens
ENYNRPA

SEQ ID NO: 40 - protein, 12 aa, Homo Sapiens
CSSWDDSLNYWV

SEQ ID NO: 41 - protein, 118 aa, Homo Sapiens
EVQLLESGGGLVQPGGSLRLSCAASGFTFSNYGMHWVRQAPGKGLEWVAVISYDGSNKYYADSVKGRFTISRDNSKNTLYLQMNSLRAEDTAVYYCAKETNDVPFDYWGQGTLVTVSS

SEQ ID NO: 42 - protein, 110 aa, Homo Sapiens
QSVLTQPPSASGTPGQRVTISCSGSSSNIGSNTVNWYQQLPGTAPKLLIYANNNRPSGVPDRFSGSKSGTSASLAISGLRSEDEADYYCAAWHDSLSGWVFGGGTKLTVL

SEQ ID NO: 43 - DNA, 354 bp, Homo Sapiens
gaggtgcagctgttggagtctgggggaggcttggtacagcctggggggtccctgagactctcctgtgcagcctctggattcaccttcagtaactatggcatgcactgggtccgccaggctccaggcaaggggctggagtgggtggcagttatatcatatgatggaagtaacaaatattatgcagactccgtgaagggtcgattcaccatctccagagacaattccaagaacacgctgtatctgcaaatgaacagcctgagagccgaggacactgccgtgtattactgtgcgaaagagactaacgacgttccctttgactactggggccaaggtaccctggtcaccgtgagctca

SEQ ID NO: 44 - DNA, 330 bp, Homo Sapiens
cagtctgtgctgactcagccaccctcagcgtctgggacccccgggcagagggtcaccatctcttgttctggaagcagctccaacatcggaagtaatactgtaaactggtatcagcagctcccaggaacggcccccaaactcctcatctatgctaacaacaatcggccctcaggggtccctgaccgattctctggctccaagtctggcacctcagcctccctggccatcagtgggctccggtccgaggatgaggctgattattactgtgcagcatggcatgacagcctgagtggttgggtgttcggcggaggaaccaagctgacggtccta

SEQ ID NO: 45 - protein, 8 aa, Homo Sapiens
FSNYGMHW

SEQ ID NO: 46 - protein, 20 aa, Homo Sapiens
VAVISYDGSNKYYADSVKGR

SEQ ID NO: 47 - protein, 11 aa, Homo Sapiens
AKETNDVPFDY

SEQ ID NO: 48 - protein, 13 aa, Homo Sapiens
SGSSSNIGSNTVN

SEQ ID NO: 49 - protein, 7 aa, Homo Sapiens
ANNNRPS

SEQ ID NO: 50 - protein, 12 aa, Homo Sapiens
CAAWHDSLSGWV

SEQ ID NO: 51 - protein, 119 aa, Homo Sapiens
EVQLLESGGGLVQPGGSLRLSCAASGFTFSNYGMYWVRQAPGKGLEWVAVISYDGTNKYYVDSVKGRFTISRDNSKNTLYLQMNSLRAEDTAVYYCAKDLPKWGGFDSWGQGTLVTVSS

SEQ ID NO: 52 - protein, 110 aa, Homo Sapiens
QSVLTQPPSASGTPGQRVTISCSGGSSNIGSNYVYWYQQLPGTAPKLLIYGNSNRPSGVPDRFSGSKSGTSASLAISGLRSEDEADYYCAAWDDSLNGVVFGGGTKLTVL

SEQ ID NO: 53 - DNA, 357 bp, Homo Sapiens
gaggtgcagctgttggagtctgggggaggcttggtacagcctggggggtccctgagactctcctgtgcagcctctggattcaccttcagtaactatggcatgtactgggtccgccaggctccaggcaaggggctggagtgggtggcagttatatcatatgatggaactaacaaatattatgtagactccgtgaagggccgattcaccatctccagagacaattccaagaacacgctgtatctgcaaatgaacagcctgagagccgaggacactgccgtgtattactgtgcaaaagatctcccgaagtggggaggctttgactcctggggccaaggtaccctggtcaccgtgagctca

SEQ ID NO: 54 - DNA, 330 bp, Homo Sapiens
cagtctgtgctgactcagccaccctcagcgtctgggacccccgggcagagggtcaccatctcttgttctggaggcagctccaacatcggaagtaattatgtatactggtatcagcagctcccaggaacggcccccaaactcctcatctatggtaacagcaatcggccctcaggggtccctgaccgattctctggctccaagtctggcacctcagcctccctggccatcagtgggctccggtccgaggatgaggctgattattactgtgcagcatgggatgacagcctgaatggtgtggtattcggcggaggaaccaagctgacggtccta

SEQ ID NO: 55 - protein, 8 aa, Homo Sapiens
FSNYGMYW

SEQ ID NO: 56 - protein, 20 aa, Homo Sapiens
VAVISYDGTNKYYVDSVKGR

SEQ ID NO: 57 - protein, 12 aa, Homo Sapiens
AKDLPKWGGFDS

SEQ ID NO: 58 - protein, 13 aa, Homo Sapiens
SGGSSNIGSNYVY

SEQ ID NO: 59 - protein, 7 aa, Homo Sapiens
GNSNRPS

SEQ ID NO: 60 - protein, 12 aa, Homo Sapiens
CAAWDDSLNGVV

SEQ ID NO: 61 - protein, 748 aa, Homo Sapiens
RPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEI

SEQ ID NO: 62 - protein, 659 aa, Homo Sapiens
RPSFSLVEDTTLEPEDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEI

SEQ ID NO: 63 - protein, 15 aa, Homo Sapiens
RPSFSLVEDTTLEPE

SEQ ID NO: 64 - protein, 15 aa, Oryctolagus cuniculus
RPSFNLVEDATVEPE

SEQ ID NO: 65 - protein, 15 aa, Sus scrofa
RPSFNLVEDTTVEPE

SEQ ID NO: 66 - protein, 15 aa, Canis familiaris
RPSFNLVEDTTLEPE

SEQ ID NO: 67 - protein, 451 aa, Homo Sapiens
EVQLLESGGGLVQPGGSLRLSCAASGFTFSSYAMSWVRQAPGKGLEWVSAISGSGTSTYYADSVKGRFTISRDNSKNTLYLQMNSLRAEDTAVYYCARVRYNWNHGDWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 216 aa, Homo Sapiens
QSVLTQPPSASGTPGQRVTISCSGSSSNIGNNYVSWYQQLPGTAPKLLIYENYNRPAGVPDRFSGSKSGTSASLAISGLRSEDEADYYCSSWDDSLNYWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 69 - protein, 447 aa, Homo Sapiens
EVQLLESGGGLVQPGGSLRLSCAASGFTFRTSGMHWVRQAPGKGLEWVAVISYDGSNKYYADSVKGRFTISRDNSKNTLYLQMNSLRAEDTAVYYCARESPGYDFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 216 aa, Homo Sapiens
QSVLTQPPSASGTPGQRVTISCSGSSSNIGSNTVNWYQQLPGTAPKLLIYGNSNRPSGVPDRFSGSKSGTSASLAISGLRSEDEADYYCAAWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 71 - protein, 451 aa, Homo Sapiens; identical to SEQ ID NO: 67

SEQ ID NO: 72 - protein, 217 aa, Homo Sapiens
AQSVLTQPPSASGTPGQRVTISCSGSSSNIGNNYVSWYQQLPGTAPKLLIYENYNRPAGVPDRFSGSKSGTSASLAISGLRSEDEADYYCSSWDDSLNYWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 73 - protein, 122 aa, Homo Sapiens; identical to SEQ ID NO: 31

SEQ ID NO: 74 - protein, 110 aa, Homo Sapiens; identical to SEQ ID NO: 32

SEQ ID NO: 75 - protein, 8 aa, Homo Sapiens
FSSYAMSW

SEQ ID NO: 76 - protein, 20 aa, Homo Sapiens
VSAISGSGTSTYYADSVKGR

SEQ ID NO: 77 - protein, 15 aa, Homo Sapiens
ARVRYNWNHGDWFDP

SEQ ID NO: 78 - protein, 13 aa, Homo Sapiens
SGSSSNIGNNYVS

SEQ ID NO: 79 - protein, 7 aa, Homo Sapiens
ENYNRPA

SEQ ID NO: 80 - protein, 12 aa, Homo Sapiens
CSSWDDSLNYWV

SEQ ID NO: 81 - protein, 451 aa, Homo Sapiens
EVQLLESGGGLVQPGGSLRLSCAASGFTFSSYAMSWVRQAPGKGLEWVSAISKSGGSTFYADSVKGRFTISRDNSKNTLYLQMNSLRAEDTAVYYCARVRYIWGEGDWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 217 aa, Homo Sapiens
AQSVLTQPPSASGTPGQRVTISCSGSSSNIGENYVSWYQQLPGTAPKLLIYENYKRSSGVPDRFSGSKSGTSASLAISGLRSEDEADYYCAAWDDSLNSWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 83 - protein, 122 aa, Homo Sapiens
EVQLLESGGGLVQPGGSLRLSCAASGFTFSSYAMSWVRQAPGKGLEWVSAISKSGGSTFYADSVKGRFTISRDNSKNTLYLQMNSLRAEDTAVYYCARVRYIWGEGDWFDPWGQGTLVTVSS

SEQ ID NO: 84 - protein, 110 aa, Homo Sapiens
QSVLTQPPSASGTPGQRVTISCSGSSSNIGENYVSWYQQLPGTAPKLLIYENYKRSSGVPDRFSGSKSGTSASLAISGLRSEDEADYYCAAWDDSLNSWVFGGGTKLTVL

SEQ ID NO: 85 - protein, 8 aa, Homo Sapiens
FSSYAMSW

SEQ ID NO: 86 - protein, 20 aa, Homo Sapiens
VSAISKSGGSTFYADSVKGR

SEQ ID NO: 87 - protein, 15 aa, Homo Sapiens
ARVRYIWGEGDWFDP

SEQ ID NO: 88 - protein, 13 aa, Homo Sapiens
SGSSSNIGENYVS

SEQ ID NO: 89 - protein, 7 aa, Homo Sapiens
ENYKRSS

SEQ ID NO: 90 - protein, 12 aa, Homo Sapiens
CAAWDDSLNSWV

SEQ ID NO: 91 - protein, 451 aa, Homo Sapiens
EVQLLESGGGLVQPGGSLRLSCAASGFTFSSYAMSWVRQAPGKGLEWVSAISRSGGSTYYADSVKGRFTISRDNSKNTLYLQMNSLRAEDTAVYYCARVRYIWNEGDWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 217 aa, Homo Sapiens
AQSVLTQPPSASGTPGQRVTISCSGSSSNIGENYVSWYQQLPGTAPKLLIYENYNRPSGVPDRFSGSKSGTSASLAISGLRSEDEADYYCAAWDDSLNSWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 93 - protein, 122 aa, Homo Sapiens
EVQLLESGGGLVQPGGSLRLSCAASGFTFSSYAMSWVRQAPGKGLEWVSAISRSGGSTYYADSVKGRFTISRDNSKNTLYLQMNSLRAEDTAVYYCARVRYIWNEGDWFDPWGQGTLVTVSS

SEQ ID NO: 94 - protein, 110 aa, Homo Sapiens
QSVLTQPPSASGTPGQRVTISCSGSSSNIGENYVSWYQQLPGTAPKLLIYENYNRPSGVPDRFSGSKSGTSASLAISGLRSEDEADYYCAAWDDSLNSWVFGGGTKLTVL

SEQ ID NO: 95 - protein, 8 aa, Homo Sapiens
FSSYAMSW

SEQ ID NO: 96 - protein, 20 aa, Homo Sapiens
VSAISRSGGSTYYADSVKGR

SEQ ID NO: 97 - protein, 15 aa, Homo Sapiens
ARVRYIWNEGDWFDP

SEQ ID NO: 98 - protein, 13 aa, Homo Sapiens
SGSSSNIGENYVS

SEQ ID NO: 99 - protein, 7 aa, Homo Sapiens
ENYNRPS

SEQ ID NO: 100 - protein, 12 aa, Homo Sapiens
CAAWDDSLNSWV

SEQ ID NO: 101 - protein, 451 aa, Homo Sapiens
EVQLLESGGGLVQPGGSLRLSCAASGFTFSSYAMSWVRQAPGKGLEWVSAISKSGGSTFYADSVKGRFTISRDNSKNTLYLQMNSLRAEDTAVYYCARVRYKWGEGDWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 217 aa, Homo Sapiens
AQSVLTQPPSASGTPGQRVTISCSGSSSNIGENYVSWYQQLPGTAPKLLIYENYKRPSGVPDRFSGSKSGTSASLAISGLRSEDEADYYCAAWDDSGNSWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 103 - protein, 122 aa, Homo Sapiens
EVQLLESGGGLVQPGGSLRLSCAASGFTFSSYAMSWVRQAPGKGLEWVSAISKSGGSTFYADSVKGRFTISRDNSKNTLYLQMNSLRAEDTAVYYCARVRYKWGEGDWFDPWGQGTLVTVSS

SEQ ID NO: 104 - protein, 110 aa, Homo Sapiens
QSVLTQPPSASGTPGQRVTISCSGSSSNIGENYVSWYQQLPGTAPKLLIYENYKRPSGVPDRFSGSKSGTSASLAISGLRSEDEADYYCAAWDDSGNSWVFGGGTKLTVL

SEQ ID NO: 105 - protein, 8 aa, Homo Sapiens
FSSYAMSW

SEQ ID NO: 106 - protein, 20 aa, Homo Sapiens
VSAISKSGGSTFYADSVKGR

SEQ ID NO: 107 - protein, 15 aa, Homo Sapiens
ARVRYKWGEGDWFDP

SEQ ID NO: 108 - protein, 13 aa, Homo Sapiens
SGSSSNIGENYVS

SEQ ID NO: 109 - protein, 7 aa, Homo Sapiens
ENYKRPS

SEQ ID NO: 110 - protein, 12 aa, Homo Sapiens
CAAWDDSGNSWV

SEQ ID NO: 111 - protein, 451 aa, Homo Sapiens; identical to SEQ ID NO: 91

SEQ ID NO: 112 - protein, 217 aa, Homo Sapiens
AQSVLTQPPSASGTPGQRVTISCSGSSSNIGNNYVSWYQQLPGTAPKLLIYENYKRPSGVPDRFSGSKSGTSASLAISGLRSEDEADYYCAAWDDSLPGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 113 - protein, 122 aa, Homo Sapiens; identical to SEQ ID NO: 93

SEQ ID NO: 114 - protein, 110 aa, Homo Sapiens
QSVLTQPPSASGTPGQRVTISCSGSSSNIGNNYVSWYQQLPGTAPKLLIYENYKRPSGVPDRFSGSKSGTSASLAISGLRSEDEADYYCAAWDDSLPGWVFGGGTKLTVL

SEQ ID NO: 115 - protein, 8 aa, Homo Sapiens
FSSYAMSW

SEQ ID NO: 116 - protein, 20 aa, Homo Sapiens
VSAISRSGGSTYYADSVKGR

SEQ ID NO: 117 - protein, 15 aa, Homo Sapiens
ARVRYIWNEGDWFDP

SEQ ID NO: 118 - protein, 13 aa, Homo Sapiens
SGSSSNIGNNYVS

SEQ ID NO: 119 - protein, 7 aa, Homo Sapiens
ENYKRPS

SEQ ID NO: 120 - protein, 12 aa, Homo Sapiens
CAAWDDSLPGWV

SEQ ID NO: 121 - protein, 451 aa, Homo Sapiens
EVQLLESGGGLVQPGGSLRLSCAASGFTFSSYAMSWVRQAPGKGLEWVSAISKSGGSTFYADSVKGRFTISRDNSKNTLYLQMNSLRAEDTAVYYCARVRYIWNEGDWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 122 - protein, 217 aa, Homo Sapiens
AQSVLTQPPSASGTPGQRVTISCSGSSSNIGENYVSWYQQLPGTAPKLLIYENYKRPSGVPDRFSGSKSGTSASLAISGLRSEDEADYYCAAWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 123 - protein, 122 aa, Homo Sapiens
EVQLLESGGGLVQPGGSLRLSCAASGFTFSSYAMSWVRQAPGKGLEWVSAISKSGGSTFYADSVKGRFTISRDNSKNTLYLQMNSLRAEDTAVYYCARVRYIWNEGDWFDPWGQGTLVTVSS

SEQ ID NO: 124 - protein, 110 aa, Homo Sapiens
QSVLTQPPSASGTPGQRVTISCSGSSSNIGENYVSWYQQLPGTAPKLLIYENYKRPSGVPDRFSGSKSGTSASLAISGLRSEDEADYYCAAWDDSLNGWVFGGGTKLTVL

SEQ ID NO: 125 - protein, 8 aa, Homo Sapiens
FSSYAMSW

SEQ ID NO: 126 - protein, 20 aa, Homo Sapiens
VSAISKSGGSTFYADSVKGR

SEQ ID NO: 127 - protein, 15 aa, Homo Sapiens
ARVRYIWNEGDWFDP

SEQ ID NO: 128 - protein, 13 aa, Homo Sapiens
SGSSSNIGENYVS

SEQ ID NO: 129 - protein, 7 aa, Homo Sapiens
ENYKRPS

SEQ ID NO: 130 - protein, 12 aa, Homo Sapiens
CAAWDDSLNGWV

SEQ ID NO: 131 - protein, 451 aa, Homo Sapiens
EVQLLESGGGLVQPGGSLRLSCAASGFTFSSYAMSWVRQAPGKGLEWVSAISKSGGSTFYADSVKGRFTISRDNSKNTLYLQMNSLRAEDTAVYYCARVRYIWNHGDWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 217 aa, Homo Sapiens
AQSVLTQPPSASGTPGQRVTISCSGSSSNIGENYVSWYQQLPGTAPKLLIYENYKRPSGVPDRFSGSKSGTSASLAISGLRSEDEADYYCAAWDDSLNSWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 133 - protein, 122 aa, Homo Sapiens
EVQLLESGGGLVQPGGSLRLSCAASGFTFSSYAMSWVRQAPGKGLEWVSAISKSGGSTFYADSVKGRFTISRDNSKNTLYLQMNSLRAEDTAVYYCARVRYIWNHGDWFDPWGQGTLVTVSS

SEQ ID NO: 134 - protein, 110 aa, Homo Sapiens
QSVLTQPPSASGTPGQRVTISCSGSSSNIGENYVSWYQQLPGTAPKLLIYENYKRPSGVPDRFSGSKSGTSASLAISGLRSEDEADYYCAAWDDSLNSWVFGGGTKLTVL

SEQ ID NO: 135 - protein, 8 aa, Homo Sapiens
FSSYAMSW

SEQ ID NO: 136 - protein, 20 aa, Homo Sapiens
VSAISKSGGSTFYADSVKGR

SEQ ID NO: 137 - protein, 15 aa, Homo Sapiens
ARVRYIWNHGDWFDP

SEQ ID NO: 138 - protein, 13 aa, Homo Sapiens
SGSSSNIGENYVS

SEQ ID NO: 139 - protein, 7 aa, Homo Sapiens
ENYKRPS

SEQ ID NO: 140 - protein, 12 aa, Homo Sapiens
CAAWDDSLNSWV

SEQ ID NO: 141 - protein, 447 aa, Homo Sapiens; identical to SEQ ID NO: 69

SEQ ID NO: 142 - protein, 217 aa, Homo Sapiens
AQSVLTQPPSASGTPGQRVTISCSGSSSNIGSNTVNWYQQLPGTAPKLLIYGNSNRPSGVPDRFSGSKSGTSASLAISGLRSEDEADYYCAAWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 143 - protein, 118 aa, Homo Sapiens; identical to SEQ ID NO: 1

SEQ ID NO: 144 - protein, 110 aa, Homo Sapiens; identical to SEQ ID NO: 22

SEQ ID NO: 145 - protein, 8 aa, Homo Sapiens
FRTSGMHW

SEQ ID NO: 146 - protein, 20 aa, Homo Sapiens
VAVISYDGSNKYYADSVKGR

SEQ ID NO: 147 - protein, 11 aa, Homo Sapiens
ARESPGYDFEY

SEQ ID NO: 148 - protein, 13 aa, Homo Sapiens
SGSSSNIGSNTVN

SEQ ID NO: 149 - protein, 7 aa, Homo Sapiens
GNSNRPS

SEQ ID NO: 150 - protein, 12 aa, Homo Sapiens
CAAWDDSLNGWV

SEQ ID NO: 151 - protein, 447 aa, Homo Sapiens
EVQLLESGGGLVQPGGSLRLSCAASGFTFSHSGMHWVRQAPGKGLEWVAVISYDGSNKYYADSVKGRFTISRDNSKNTLYLQMNSLRAEDTAVYYCAREQPLYDFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 152 - protein, 217 aa, Homo Sapiens
AQSVLTQPPSASGTPGQRVTISCSGSSSNIGDNGVNWYQQLPGTAPKLLIYGASERPSGVPDRFSGSKSGTSASLAISGLRSEDEADYYCAAWDDSLDGWTFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 153 - protein, 118 aa, Homo Sapiens
EVQLLESGGGLVQPGGSLRLSCAASGFTFSHSGMHWVRQAPGKGLEWVAVISYDGSNKYYADSVKGRFTISRDNSKNTLYLQMNSLRAEDTAVYYCAREQPLYDFEYWGQGTLVTVSS

SEQ ID NO: 154 - protein, 110 aa, Homo Sapiens
QSVLTQPPSASGTPGQRVTISCSGSSSNIGDNGVNWYQQLPGTAPKLLIYGASERPSGVPDRFSGSKSGTSASLAISGLRSEDEADYYCAAWDDSLDGWTFGGGTKLTVL

SEQ ID NO: 155 - protein, 8 aa, Homo Sapiens
FSHSGMHW

SEQ ID NO: 156 - protein, 20 aa, Homo Sapiens
VAVISYDGSNKYYADSVKGR

SEQ ID NO: 157 - protein, 11 aa, Homo Sapiens
AREQPLYDFEY

SEQ ID NO: 158 - protein, 13 aa, Homo Sapiens
SGSSSNIGDNGVN

SEQ ID NO: 159 - protein, 7 aa, Homo Sapiens
GASERPS

SEQ ID NO: 160 - protein, 12 aa, Homo Sapiens
CAAWDDSLDGWT

SEQ ID NO: 161 - protein, 447 aa, Homo Sapiens; identical to SEQ ID NO: 151

SEQ ID NO: 162 - protein, 217 aa, Homo Sapiens
AQSVLTQPPSASGTPGQRVTISCSGSSSNIGDNTVNWYQQLPGTAPKLLIYEASERPSGVPDRFSGSKSGTSASLAISGLRSEDEADYYCAAWDDSLDGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 163 - protein, 118 aa, Homo Sapiens; identical to SEQ ID NO: 153

SEQ ID NO: 164 - protein, 110 aa, Homo Sapiens
QSVLTQPPSASGTPGQRVTISCSGSSSNIGDNTVNWYQQLPGTAPKLLIYEASERPSGVPDRFSGSKSGTSASLAISGLRSEDEADYYCAAWDDSLDGWVFGGGTKLTVL

SEQ ID NO: 165 - protein, 8 aa, Homo Sapiens
FSHSGMHW

SEQ ID NO: 166 - protein, 20 aa, Homo Sapiens
VAVISYDGSNKYYADSVKGR

SEQ ID NO: 167 - protein, 11 aa, Homo Sapiens
AREQPLYDFEY

SEQ ID NO: 168 - protein, 13 aa, Homo Sapiens
SGSSSNIGDNTVN

SEQ ID NO: 169 - protein, 7 aa, Homo Sapiens
EASERPS

SEQ ID NO: 170 - protein, 12 aa, Homo Sapiens
CAAWDDSLDGWV

SEQ ID NO: 171 - protein, 447 aa, Homo Sapiens
EVQLLESGGGLVQPGGSLRLSCAASGFTFSHSGMHWVRQAPGKGLEWVAVISYDGSNKYYADSVKGRFTISRDNSKNTLYLQMNSLRAEDTAVYYCAREQPVYDFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 172 - protein, 217 aa, Homo Sapiens
AQSVLTQPPSASGTPGQRVTISCSGSSSNIGDNTVNWYQQLPGTAPKLLIYEASNRPSGVPDRFSGSKSGTSASLAISGLRSEDEADYYCAAWDDSLNGWTFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 173 - protein, 118 aa, Homo Sapiens
EVQLLESGGGLVQPGGSLRLSCAASGFTFSHSGMHWVRQAPGKGLEWVAVISYDGSNKYYADSVKGRFTISRDNSKNTLYLQMNSLRAEDTAVYYCAREQPVYDFEYWGQGTLVTVSS

SEQ ID NO: 174 - protein, 110 aa, Homo Sapiens
QSVLTQPPSASGTPGQRVTISCSGSSSNIGDNTVNWYQQLPGTAPKLLIYEASNRPSGVPDRFSGSKSGTSASLAISGLRSEDEADYYCAAWDDSLNGWTFGGGTKLTVL

SEQ ID NO: 175 - protein, 8 aa, Homo Sapiens
FSHSGMHW

SEQ ID NO: 176 - protein, 20 aa, Homo Sapiens
VAVISYDGSNKYYADSVKGR

SEQ ID NO: 177 - protein, 11 aa, Homo Sapiens
AREQPVYDFEY

SEQ ID NO: 178 - protein, 13 aa, Homo Sapiens
SGSSSNIGDNTVN

SEQ ID NO: 179 - protein, 7 aa, Homo Sapiens
EASNRPS

SEQ ID NO: 180 - protein, 12 aa, Homo Sapiens
CAAWDDSLNGWT

SEQ ID NO: 181 - protein, 447 aa, Homo Sapiens
EVQLLESGGGLVQPGGSLRLSCAASGFTFSTSGMHWVRQAPGKGLEWVAVISYDGSNKYYADSVKGRFTISRDNSKNTLYLQMNSLRAEDTAVYYCARESPGYDFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 182 - protein, 217 aa, Homo Sapiens
AQSVLTQPPSASGTPGQRVTISCSGSSSNIGDNGVNWYQQLPGTAPKLLIYGASERPSGVPDRFSGSKSGTSASLAISGLRSEDEADYYCAAWDDSLDGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 183 - protein, 118 aa, Homo Sapiens
EVQLLESGGGLVQPGGSLRLSCAASGFTFSTSGMHWVRQAPGKGLEWVAVISYDGSNKYYADSVKGRFTISRDNSKNTLYLQMNSLRAEDTAVYYCARESPGYDFEYWGQGTLVTVSS

SEQ ID NO: 184 - protein, 110 aa, Homo Sapiens
QSVLTQPPSASGTPGQRVTISCSGSSSNIGDNGVNWYQQLPGTAPKLLIYGASERPSGVPDRFSGSKSGTSASLAISGLRSEDEADYYCAAWDDSLDGWVFGGGTKLTVL

SEQ ID NO: 185 - protein, 8 aa, Homo Sapiens
FSTSGMHW

SEQ ID NO: 186 - protein, 20 aa, Homo Sapiens
VAVISYDGSNKYYADSVKGR

SEQ ID NO: 187 - protein, 11 aa, Homo Sapiens
ARESPGYDFEY

SEQ ID NO: 188 - protein, 13 aa, Homo Sapiens
SGSSSNIGDNGVN

SEQ ID NO: 189 - protein, 7 aa, Homo Sapiens
GASERPS

SEQ ID NO: 190 - protein, 12 aa, Homo Sapiens
CAAWDDSLDGWV

SEQ ID NO: 191 - protein, 447 aa, Homo Sapiens
EVQLLESGGGLVQPGGSLRLSCAASGFTFSHSGMHWVRQAPGKGLEWVAVISYDGSNKYYADSVKGRFTISRDNSKNTLYLQMNSLRAEDTAVYYCAREAPGYDFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 192 - protein, 217 aa, Homo Sapiens
AQSVLTQPPSASGTPGQRVTISCSGSSSNIGDNTVNWYQQLPGTAPKLLIYGASERPSGVPDRFSGSKSGTSASLAISGLRSEDEADYYCAAWDDSLNGWTFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 193 - protein, 118 aa, Homo Sapiens
EVQLLESGGGLVQPGGSLRLSCAASGFTFSHSGMHWVRQAPGKGLEWVAVISYDGSNKYYADSVKGRFTISRDNSKNTLYLQMNSLRAEDTAVYYCAREAPGYDFEYWGQGTLVTVSS

SEQ ID NO: 194 - protein, 110 aa, Homo Sapiens
QSVLTQPPSASGTPGQRVTISCSGSSSNIGDNTVNWYQQLPGTAPKLLIYGASERPSGVPDRFSGSKSGTSASLAISGLRSEDEADYYCAAWDDSLNGWTFGGGTKLTVL

SEQ ID NO: 195 - protein, 8 aa, Homo Sapiens
FSHSGMHW

SEQ ID NO: 196 - protein, 20 aa, Homo Sapiens
VAVISYDGSNKYYADSVKGR

SEQ ID NO: 197 - protein, 11 aa, Homo Sapiens
AREAPGYDFEY

SEQ ID NO: 198 - protein, 13 aa, Homo Sapiens
SGSSSNIGDNTVN

SEQ ID NO: 199 - protein, 7 aa, Homo Sapiens
GASERPS

SEQ ID NO: 200 - protein, 12 aa, Homo Sapiens
CAAWDDSLNGWT

SEQ ID NO: 201 - protein, 447 aa, Homo Sapiens
EVQLLESGGGLVQPGGSMRLSCAASGFTFSSSGMHWVRQAPGKGLEWVAVISYDGSNKYYADSVKGRFTISRDNSKNTLYLQMNSLRAEDTAVYYCAREQPGYDFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 202 - protein, 217 aa, Homo Sapiens
AQSVLTQPPSASGTPGQRVTISCSGSSSNIGDNGVNWYQQLPGTAPKLLIYEASERPSGVPDRFSGSKSGTSASLAISGLRSEDEADYYCAAWDDSLDGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 203 - protein, 118 aa, Homo Sapiens
EVQLLESGGGLVQPGGSMRLSCAASGFTFSSSGMHWVRQAPGKGLEWVAVISYDGSNKYYADSVKGRFTISRDNSKNTLYLQMNSLRAEDTAVYYCAREQPGYDFEYWGQGTLVTVSS

SEQ ID NO: 204 - protein, 110 aa, Homo Sapiens
QSVLTQPPSASGTPGQRVTISCSGSSSNIGDNGVNWYQQLPGTAPKLLIYEASERPSGVPDRFSGSKSGTSASLAISGLRSEDEADYYCAAWDDSLDGWVFGGGTKLTVL

SEQ ID NO: 205 - protein, 8 aa, Homo Sapiens
FSSSGMHW

SEQ ID NO: 206 - protein, 20 aa, Homo Sapiens
VAVISYDGSNKYYADSVKGR

SEQ ID NO: 207 - protein, 11 aa, Homo Sapiens
AREQPGYDFEY

SEQ ID NO: 208 - protein, 13 aa, Homo Sapiens
SGSSSNIGDNGVN

SEQ ID NO: 209 - protein, 7 aa, Homo Sapiens
EASERPS

SEQ ID NO: 210 - protein, 12 aa, Homo Sapiens
CAAWDDSLDGWV

SEQ ID NO: 211 - protein, 447 aa, Homo Sapiens
EVQLLESGGGLVQPGGSLRLSCAASGFTFSHSGMHWVRQAPGKGLEWVAVISYDGSNKYYADSVKGRFTISRDNSKNTLYLQMNSLRAEDTAVYYCAREAPLYDFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 212 - protein, 217 aa, Homo Sapiens
AQSVLTQPPSASGTPGQRVTISCSGSSSNIGDNGVNWYQQLPGTAPKLLIYGASNRPSGVPDRFSGSKSGTSASLAISGLRSEDEADYYCAAWDDSLDGWVFGGGTKLTVLGQPKAAPSVTLFPPSSEELQANKATLVCLISDFCPGAVTVAWKADSSPVKAGVETTTPSKQSNNKYAASSYLSLTPEQWKSHRSYSCQVTHEGSTVEKTVAPTECS

SEQ ID NO: 213 - protein, 118 aa, Homo Sapiens
EVQLLESGGGLVQPGGSLRLSCAASGFTFSHSGMHWVRQAPGKGLEWVAVISYDGSNKYYADSVKGRFTISRDNSKNTLYLQMNSLRAEDTAVYYCAREAPLYDFEYWGQGTLVTVSS

SEQ ID NO: 214 - protein, 110 aa, Homo Sapiens
QSVLTQPPSASGTPGQRVTISCSGSSSNIGDNGVNWYQQLPGTAPKLLIYGASNRPSGVPDRFSGSKSGTSASLAISGLRSEDEADYYCAAWDDSLDGWVFGGGTKLTVL

SEQ ID NO: 215 - protein, 8 aa, Homo Sapiens
FSHSGMHW

SEQ ID NO: 216 - protein, 20 aa, Homo Sapiens
VAVISYDGSNKYYADSVKGR

SEQ ID NO: 217 - protein, 11 aa, Homo Sapiens
AREAPLYDFEY

SEQ ID NO: 218 - protein, 13 aa, Homo Sapiens
SGSSSNIGDNGVN

SEQ ID NO: 219 - protein, 7 aa, Homo Sapiens
GASNRPS

SEQ ID NO: 220 - protein, 12 aa, Homo Sapiens
CAAWDDSLDGWV

SEQ ID NO: 221 - protein, 452 aa, Homo Sapiens
EVQLLESGGGLVQPGGSLRLSCAASGFTFSSYAMSWVRQAPGKGLEWVSAISGSGTSTYYADSVKGRFTISRDNSKNTLYLQMNSLRAEDTAVYYCARVRYNWNHGDWFDPWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 222 - protein, 452 aa, Homo Sapiens
QVELLESGGGLVQPGGSLRLSCAASGFTFSSYAMSWVRQAPGKGLEWVSAISGSGTSTYYADSVKGRFTISRDNSKNTLYLQMNSLRAEDTAVYYCARVRYNWNHGDWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK